(19) United States
(12) Patent Application Publication
(10) Pub. No.: US 2024/0229056 A1
(43) Pub. Date: Jul. 11, 2024

(54) TOXIN-FREE ALOE AND METHODS OF MAKING SAME

(71) Applicant: Chi Botanic Inc, Alameda, CA (US)

(72) Inventors: Jonathan E. Meuser, Alameda, CA (US); Robert Edward Jinkerson, Florissant, MO (US)

(21) Appl. No.: 18/557,916

(22) PCT Filed: Apr. 29, 2022
(86) PCT No.: PCT/US2022/026922

§ 371 Date: Oct. 27, 2023

Related U.S. Application Data
(60) Provisional application No. 63/182,468, filed on Apr. 30, 2021.

(51) Int. Cl. C12N 15/82; C12N 5/04; C12N 9/22; C12N 15/11; G01N 21/64; G01N 33/00

(52) U.S. Cl. CPC C12N 15/8243; C12N 5/04; C12N 9/22; C12N 15/11; C12N 15/8213; G01N 21/6486; G01N 33/0098; C12N 2310/20; G01N 2333/91057

(57) ABSTRACT

Toxin-free mutant aloe has attenuated expression of at least one gene involved in the biosynthesis of a natural toxin, such as aloin. Methods of producing toxin-free mutant aloe involve obtaining plant cells derived from a species of aloe and attenuating the expression level of at least one polyketide synthase gene within the obtained plant cells to form mutant plant cells. Methods further involve culturing the mutant plant cells under a set of culture conditions conducive to proliferation of the mutant plant cells. Methods further involve growing the mutant plant cells in a bioreactor and harvesting a toxin-free aloe bioproduct therefrom. Attenuating the expression level of at least one polyketide synthase gene involves disrupting the gene with a targeted endonuclease system and selecting the successfully mutated cells prior to cell culturing and expansion.

### CROSS-REFERENCE TO RELATED APPLICATIONS

This application claims priority to U.S. Provisional Patent Application No. 63/182,468, filed Apr. 30, 2021, entitled “TOXIN-FREE ALOE AND METHODS OF MAKING SAME,” which is incorporated by reference herein, in the entirety and for all purposes.

### TECHNICAL FIELD

Implementations relate to aloe mutants and associated methods of production. Specific implementations provide scalable, high-throughput methods of producing aloe mutants lacking one or more naturally produced toxins, such as aloin. Toxin-free aloe mutants disclosed herein can have significantly reduced expression levels of one or more genes required for toxin biosynthesis.

### SEQUENCE LISTING

The instant application contains a Sequence Listing which has been submitted electronically in ASCII format and is hereby incorporated by reference in its entirety. Said ASCII copy, created on Apr. 30, 2021, is named P291962_US_01_SL.txt and is 264,033 bytes in size.

### BACKGROUND

A wide variety of biomedical, pharmaceutical, and cosmetic products include aloe, which is typically harvested from succulent plants of the species Aloe vera. The use of aloe-based products as home remedies for treating sunburn, healing wounds, and improving immune function, for instance, is particularly common. While widely popular, the production of consumer-safe aloe products requires time-consuming and expensive processing steps to remove the harmful toxins produced naturally by aloe plants. Therefore extensive efforts, such as manual separation of the inner leaf from the rind and charcoal filtration, are undertaken to eliminate toxic and bitter compounds from the highly valuable inner leaf gel. Traditional agricultural practices, which often involve substantial resource expenditures to nurture the slow-growing aloe plants on large plots of farmland, further impede the accelerated rate of scalable aloe production needed to meet a rising global demand. These problems will only become increasingly difficult to address as available farmland shrinks, natural resources become more scarce, and the negative environmental impacts caused by fertilizers, herbicides, and pesticides continue to worsen. Novel, sustainable methods of more efficiently producing toxin-free aloe at an industrial scale are needed.

### SUMMARY

The present disclosure provides novel methods, systems, and reagents for producing toxin-free aloe cells, cell cultures, seeds, and plants. Particular embodiments involve eliminating, suppressing or otherwise attenuating the expression of one or more genes that code for one or more proteins, which may be biosynthetically upstream or otherwise implicated in the expression of at least one natural aloe toxin, such as aloin. Specific examples can involve attenuating the expression of the aloe polyketide synthase 1 and/or 2 genes (“pks1 and/or pks2”). The resulting toxin-free aloe, which cannot be made naturally by unmodified aloe plants, may be cultured under novel conditions and expanded using industrial-scale bioreactors, from which toxin-free aloe cells can be harvested. These cells may also be transferred to a solid medium to generate toxin-free aloe plants for the field.

According to embodiments described herein, mutant aloe can exhibit attenuated expression of at least one gene encoding a protein biosynthetically upstream of aloin by one or more gene disruption techniques including, for example, CRISPR editing, meganuclease activity, RNA interference, and/or ribozyme activity. For instance, the gene disruption may be effected by CRISPR RNA-guided endonuclease cleavage, insertional mutagenesis, total or partial gene deletion, non-homologous end joining, and/or homologous recombination. For CRISPR-mediated DNA cleavage, the endonuclease may be Cas9 or Cbf1. In various embodiments, the attenuated expression of the pks1 and/or pks2 genes along with the PKS1 and/or PKS2 proteins in the mutant aloe achieved via one or more of the aforementioned techniques, as measured by RNA transcription and/or amino acid translation, can be less than 50%, 45%, 40%, 35%, 30%, 25%, 20%, 15%, 10%, 5%, 3%, or 1% of the amount of RNA transcribed and/or amino acids translated in wild-type aloe. In some examples, the amount of RNA transcribed and/or amino acids translated from the attenuated pks1 and/or pks2 genes may be undetectable or not significantly increased above background levels compared to the RNA transcribed and/or the amino acids translated from the corresponding pks1 and/or pks2 genes in wild-type aloe.

In accordance with embodiments of the present disclosure, a method of producing a mutant aloe can involve obtaining plant cells derived from a species of aloe, attenuating an expression level of at least one protein required for biosynthesis of a toxin produced by wild-type aloe in the obtained plant cells to form mutant plant cells, and culturing the mutant plant cells under a set of culture conditions.

In some examples, the method can further involve converting the obtained plant cells into plant stem cells prior to attenuating the expression level of at least one protein required for biosynthesis of the toxin. In some examples, the method can further involve growing the cultured mutant plant cells in a bioreactor. In some examples, the bioreactor has a volume of at least 50 liters. In some examples, the method further involves harvesting an aloe bioproduct from the cultured mutant plant cells. In some examples, the toxin comprises aloin. In some examples, the method further involves regenerating an aloe plant using the mutant plant cells. In some examples, the method further involves selecting the mutant plant cells before culturing the mutant plant cells under a set of culture conditions. In some examples, selecting the mutant plant cells involves screening the mutant plant cells for fluorescence. In some examples, selecting the mutant plant cells involves selecting fluorescence-free plant cells. In some examples, selecting the mutant plant cells involves applying one or more antibiotics to the mutant plant cells.

In some examples, attenuating an expression level of at least one protein required for biosynthesis of a toxin involves disrupting a function of the at least one protein. In some examples, the function includes an enzymatic activity. In some examples, the at least one protein comprises polyketide synthase 1 (PKS1) and/or polyketide synthase 2 (PKS2). In some examples, the PKS1 protein has an amino acid sequence having at least 55% identity to SEQ ID NO: 1. In some examples, the PKS2 protein has an amino acid sequence having at least 55% identity to SEQ ID NO: 2. In some examples, attenuating the expression level of at least one protein involves disrupting expression of the pks1 and/or pks2 gene. In some examples, disrupting expression of the pks1 and/or pks2 gene involves transforming bacterial cells with an endonuclease configured to cleave pks1 and/or pks2 and infecting the obtained plant cells with the transformed bacterial cells. In some examples, the bacterial cells are further transformed with a ribonucleoprotein. In some examples, the endonuclease is encoded in a CRISPR construct. In some examples, a guide RNA sequence included in the CRISPR construct has a sequence having at least 55% identity to SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10. In some examples, the bacterial cells are derived from Agrobacteria tumefaciens, Ensifer adhaerens, Ochrobactrum haywardense, Rhizobium etli, Sinorhizobium meliloti, Mesorhizobium loti, Rhizobium sp. strain NGR234, or a combination thereof.

In some examples, disrupting expression of the pks1 and/or pks2 gene involves delivering an endonuclease and/or ribonucleoprotein targeting pks1 and/or pks2 to the obtained plant cells via particle bombardment. In some examples, disrupting expression of the pks1 and/or pks2 gene involves delivering an endonuclease and/or ribonucleoprotein targeting pks1 and/or pks2 to the obtained plant cells using carbon nanotubes, silicon carbide whiskers, direct injection, or a combination thereof.

In accordance with some embodiments of the present disclosure, a method of producing a mutant aloe plant involves obtaining plant cells derived from a species of aloe, attenuating an expression level of at least one protein required for biosynthesis of a toxin produced by wild-type aloe in the obtained plant cells to form mutant plant cells, and using the mutant plant cells to regenerate the mutant aloe plant, from which genetically modified seeds may be subsequently obtained.

In some examples, the toxin includes aloin. In some examples, attenuating an expression level of at least one protein required for biosynthesis of a toxin involves disrupting a function of the at least one protein. In some examples, the at least one protein includes polyketide synthase 1 (PKS1) and/or polyketide synthase 2 (PKS2). In some examples, attenuating the expression level of at least one protein involves disrupting expression of the pks1 and/or pks2 gene. In some examples, disrupting expression of the pks1 and/or pks2 gene involves transforming bacterial cells with an endonuclease configured to cleave pks1 and/or pks2 and infecting the obtained plant cells with the transformed bacterial cells.

In some examples, the method further involves culturing the mutant plant cells under a set of culture conditions. In some examples, the endonuclease is encoded in a CRISPR construct.

In accordance with some embodiments of the present disclosure, a mutant aloe has attenuated expression of at least one polyketide synthase (pks) gene. In some examples, the at least one pks gene comprises pks1 and/or pks2. In some examples, an amount of mRNA transcribed by pks1 and/or pks2 is less than 10% of an amount of mRNA transcribed by pks1 and/or pks2 in wild-type aloe. In some examples, the mutant aloe comprises a mutant aloe cell. In some examples, the mutant aloe comprises a mutant aloe cell culture. In some examples, the mutant aloe comprises mutant Aloe vera. In some examples, the mutant aloe comprises a regenerated aloe plant. In some examples, the mutant aloe comprises a mutant aloe bioproduct comprising or included in one or more of: an aloe-based gel, cream, lotion, soap, sunscreen, spray, haircare product, jelly, moisturizer, cleanser, toner, skin treatment composition, cosmetic, mouthwash, toothpaste, edible food or liquid. In some examples, the mutant aloe comprises a mutant aloe seed. In some examples, the mutant aloe exhibits an absence of one or more toxins produced by wild-type aloe. In some examples, the one or more toxins include aloin. In some examples, an amount of aloin produced in the mutant aloe is less than 10% of an amount of aloin produced in wild-type aloe.

### BRIEF DESCRIPTION OF THE DRAWINGS

FIG. 1 is a flow diagram of a method performed in accordance with principles of the present disclosure.

FIG. 2 is a nucleic acid map showing an exogenous plasmid designed for bacterial transformation and CRISPR editing in accordance with principles of the present disclosure.

FIG. 3 is a flow diagram of another method performed in accordance with principles of the present disclosure.

### DETAILED DESCRIPTION

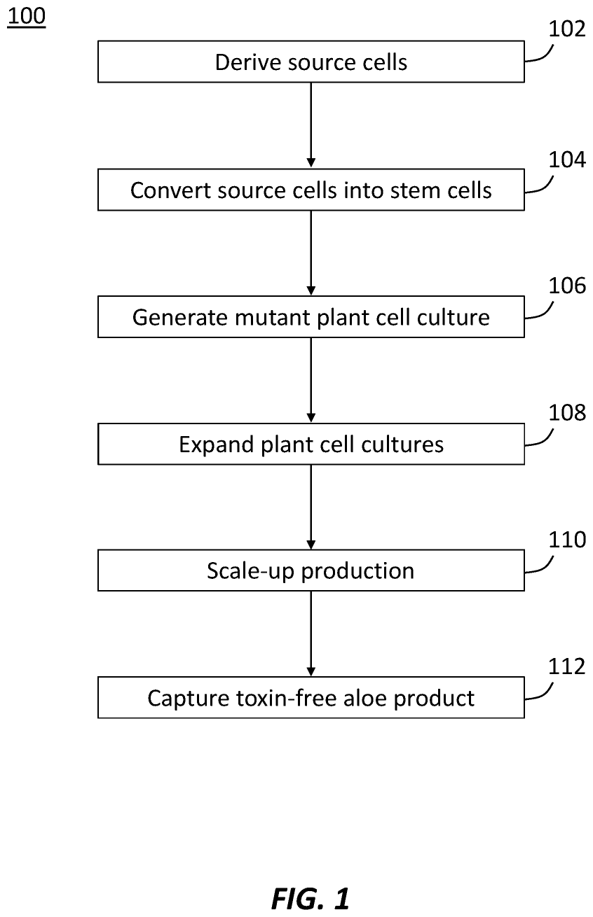

Systems, methods, and reagents for generating toxin-free or substantially toxin-free aloe mutants are disclosed herein. As shown in the method 100 of FIG. 1, the disclosed methods may generally involve obtaining aloe plant cells 102, e.g., wild-type aloe plant cells, converting the obtained cells into stem cells 104, and generating mutant plant cell cultures 106 by genetically modifying the stem cells to attenuate the production of at least one naturally produced aloe toxin. The toxin-free cell cultures can then be expanded 108 and scaled 110 to industrial volumes in tightly controlled bioreactors, from which toxin-free aloe products can eventually be harvested 112. By using specifically programmed, genetically modified cell cultures selected for toxin suppression, the disclosed methods can significantly accelerate aloe production rates and minimize waste by selectively growing only the cell types necessary to produce harvestable aloe. The disclosed approaches can also generate toxin-free aloe at an industrial scale without the need for vast areas of farmland, thereby lowering costs to consumers and the environment, and providing an efficient aloe production system untethered from geography, climate, and season.

At least one toxin suppressed according to the methods described herein can include aloin, which is a natural glycoside produced by many species of aloe plants. The incorporation of aloin in medicinal and cosmetic products is potentially harmful, especially when such products are applied directly to the skin or ingested orally. Multiple forms and types of aloin may be produced by various aloe species, including aloin A, aloin B, barbaloin, isobarbaloin, and/or aloe-emodin, at least one of which can be attenuated according to the present disclosure. For ease of illustration, these varieties are collectively referred to as “aloin” herein, and the terms “toxin-free,” “substantially toxin-free,” and/or “mutant” may refer to an aloe cell, aloe cell culture, aloe specimen, aloe seed, aloe biomass, aloe plant, or aloe plant product that lacks or substantially lacks aloin. The terms “toxin-free,” “substantially toxin-free,” or “mutant” may also refer to an aloe cell, aloe cell culture, aloe specimen, aloe seed, aloe biomass, aloe plant, or aloe plant product that lacks or substantially lacks one or more additional toxins suppressed by the techniques disclosed herein. Non-limiting examples of additionally targeted toxins can include hydroxyanthracene derivatives such as anthraquinones, aloe-emodin, and/or aloenin.

Plant-specific polyketide synthases (“PKSs”) are enzymes used to catalyze the synthesis of a variety of plant products, including polyketides, which are metabolites formed from coenzyme A (“CoA”) precursors. Embodiments disclosed herein are directed to attenuating the expression of the genes required for polyketide synthase 1 (“PKS1”) and/or polyketide synthase 2 (“PKS2”) production and/or function, such as pks1 and/or pks2. Conserved regions of PKS1 and PKS2, both of which are Type III polyketide synthases, may be nearly identical and may include at least one active site domain, at least one product binding-site domain, at least one malonyl-CoA binding site domain, and at least one dimer interface domain. Modifying the genetic sequence encoding one or more of these domains by implementing the methods disclosed herein may interfere with, or even prevent, proper PKS formation, e.g., translation and/or protein folding, and/or function, e.g., substrate binding and/or enzymatic activity. The term “conserved domain” refers to a conserved part of a given protein or DNA sequence that can evolve and/or function independently of the rest of the protein or DNA sequence. In the case of PKS domains, each wild-type domain may form a compact three-dimensional structure and often can be independently stable and folded.

Because the genomic location and sequence of the aloin synthase gene remains unknown, and because the aloin biosynthetic pathway has not been fully elucidated, reducing or eliminating functional aloin production has been elusive. The disclosed methods solve this problem by attenuating the expression of at least one gene encoding a protein that has been recognized by the inventors as necessary for aloin biosynthesis and/or function. As noted above, non-limiting examples described herein are directed to pks1 and/or pks2 attenuation, which can be achieved using one or more genetic engineering techniques disclosed herein. For example, plant cells derived from aloe plants can be infected with bacterial cells that have been transformed with at least one clustered regularly interspaced short palindromic repeat (“CRISPR”) construct designed to specifically target and suppress the expression of psk1 and/or pks2, thereby also suppressing translation of the PKS1 and/or PKS2 proteins and interfering with aloin synthesis. Cultured mutant cells exhibiting reduced or non-existent aloin production via successful psk1/2 attenuation can be selected in vitro, quickly expanded, and then scaled to industrially relevant volumes in one or more bioreactors. The benefits of scalable, cell culture-based aloe production may be enormous. For example, by implementing the processes described herein, one 50-liter bioreactor fostering growth of a toxin-free aloe cell culture may produce the amount of aloe that would otherwise require about one hectare of farmland to produce. The increased production can also be achieved up to about 30-100 times faster than plants grown under controlled conditions, thereby simultaneously reducing waste and accelerating production rates. In another embodiment, aloe plants may be regenerated from pks1/2 mutant cell lines to produce a non-toxic aloe product using traditional agricultural methods. Mutant plant seeds can be collected from the mutant aloe plants and subsequently re-planted to regenerate the plants.

The term “gene” is used broadly herein to refer to any segment of a nucleic acid molecule (typically DNA, but optionally RNA) encoding a polypeptide or expressed RNA. Genes therefore include sequences encoding expressed RNA (which can include polypeptide coding sequences or, for example, functional RNAs, microRNAs, short hairpin RNAs, ribozymes, etc.). Genes may further comprise regulatory sequences required for or affecting their expression, as well as sequences associated with the protein or RNA-encoding sequence in its natural state, such as, for example, intron sequences, 5′ or 3′ untranslated sequences, etc. In some examples, a gene may only refer to a protein-encoding portion of a DNA or RNA molecule, which may or may not include introns. A gene is preferably greater than 50 nucleotides in length, more preferably greater than 100 nucleotides in length, between about 50 and about 500,000 nucleotides in length, about 100 nucleotides, about 1,000 nucleotides, about 5,000 nucleotides, about 10,000 nucleotides, about 25,000 nucleotides in length, about 50,000 nucleotides in length, about 75,000 nucleotides in length, about 100,000 nucleotides in length, about 150,000 nucleotides in length, about 200,000 nucleotides in length, about 250,000 nucleotides in length, about 300,000 nucleotides in length, about 350,000 nucleotides in length, about 400,000 nucleotides in length, about 450,000 nucleotides in length, about 500,000 nucleotides in length, or longer, or any length therebetween.

The term “nucleic acid” or “nucleic acid molecule” refers to a segment of DNA or RNA (e.g., mRNA), and also includes nucleic acids having modified backbones (e.g., peptide nucleic acids) or modified or non-naturally-occurring nucleobases. The nucleic acid molecules can be double-stranded or single-stranded. A single-stranded nucleic acid that comprises a gene or a portion thereof can be a coding (sense) strand or a non-coding (antisense) strand.

A nucleic acid molecule may be “derived from” an indicated source, which includes the isolation (in whole or in part) of a nucleic acid segment from an indicated source. A nucleic acid molecule may also be derived from an indicated source by, for example, direct cloning, PCR amplification, or artificial synthesis from the indicated polynucleotide source or based on a sequence associated with the indicated polynucleotide source or based on a sequence associated with the indicated polynucleotide source. Genes or nucleic acid molecules derived from a particular source or species also include genes or nucleic acid molecules having sequence modifications with respect to the source nucleic acid molecules. For example, a gene or nucleic acid molecule derived from a source (e.g., a particular reference gene) can include one or more mutations with respect to the source gene or nucleic acid molecule that are unintended or that are deliberately introduced, and if one or more mutations, including substitutions, deletions, or insertions, are deliberately introduced the sequence alterations can be introduced by random or targeted mutation of cells or nucleic acids, by amplification or other molecular biology techniques, or by chemical synthesis, or any combination thereof. A gene or nucleic acid molecule that is derived from a referenced gene or nucleic molecule that encodes a functional RNA or polypeptide can encode a functional RNA or polypeptide having at least 75%, at least 80%, at least 85%, at least 90%, or at least 95% sequence identity with the referenced source functional RNA or polypeptide, or to a functional fragment thereof. For example, a gene or nucleic acid molecule that is derived from a referenced gene or nucleic acid molecule that encodes a functional RNA or polypeptide can encode a functional RNA or polypeptide having at least 85%, at least 90%, at least 95%, at least 96%, at least 97%, at least 98%, or at least 99% sequence identity with the referenced or source functional RNA or polypeptide, or to a functional fragment thereof.

The terms “naturally occurring” and “wild-type” refer to a form found in nature. For instance, a naturally occurring or wild-type nucleic acid molecule, nucleotide sequence, polypeptide, or protein may be present in and isolated from a natural source, and is not intentionally modified by human manipulation. Naturally occurring or wild-type genes and/or proteins may specifically lack one or more of the mutations created by the genetic engineering techniques implemented pursuant to the disclosed methods to produce mutant and/or toxin-free aloe.

As used herein, “attenuated” can mean suppressed, reduced, eliminated, and/or altered in amount, degree, intensity, function, and/or strength. Attenuated gene expression may refer to a significantly reduced amount and/or rate of transcription of a gene in question, e.g., pks1/2, or a reduction and/or disruption in the proper translation, folding, activity, and/or assembly of a protein, e.g., PKS1/PKS2, encoded by the gene(s). As non-limiting examples, a gene may be attenuated due to an unnatural mutation and/or unnatural disruption in the gene (e.g., a gene disruption by double-stranded cleavage, endogenous error repair, partial or total deletion, truncation, frameshifting, or insertional mutation) or may have decreased expression due to alteration, replacement, and/or elimination of one or more gene regulatory sequences. Mutant aloe cells having attenuated expression of a gene, such as a pks1 and/or pks2, can be recombinant aloe cells in which the attenuation is the result of genetic engineering, i.e., via human intervention that may involve the introduction of one or more non-native nucleic acid molecules, e.g., CRISPR plasmid constructs, or polypeptides into the aloe cells. Additionally, the attenuation may be completed in whole or in part following the introduction of an exogenous construct via endogenous DNA damage repair mechanisms, e.g., non-homologous end joining, homologous recombination, base excision repair, nucleotide excision repair, and/or mismatch repair. In addition or alternatively, gene attenuation can be achieved via other forms of mutagenesis.

An “exogenous nucleic acid molecule” or “exogenous gene” refers to a nucleic acid molecule or gene that has been introduced (e.g., “transformed” and/or “infected”) into a cell. A transformed cell may be referred to as a recombinant cell into which additional exogenous gene(s) may be introduced. A descendent of a cell transformed with a nucleic acid molecule is also referred to as “transformed” if it has inherited the exogenous nucleic acid molecule. The exogenous gene(s) may be from one or more different species (“heterologous”) or from the same species (“homologous”) relative to the cell being transformed. An “endogenous” nucleic acid molecule, gene, or protein is a native nucleic acid molecule, gene, or protein as it occurs in, or is naturally produced by, the host cell.

As used herein, “expression” includes the expression of a gene, e.g., pks1 or pks2, at least at the level of RNA production, and an “expression product” includes the resultant product, e.g., an mRNA, functional RNA, polypeptide, and/or protein of an expressed gene. The term “decreased expression” includes decreased mRNA production of a gene, either before or after mRNA processing, e.g., RNA splicing. It follows that “decreased production” refers to a decrease in protein abundance or the abundance of active protein resulting from gene expression. Decreased production of a protein, e.g., PKS1 or PKS2, includes a decrease in the amount of polypeptide expression, in the proper folding and/or dimerization, in the enzymatic activity of a polypeptide, or a combination thereof, relative to the native production, configuration, and/or enzymatic activity of the polypeptide. Accordingly, attenuated pks1 and/or pks2 expression may encompass normal or substantially normal expression levels of the gene(s), but which ultimately results in the translation of non-functional and/or altered PKS1 and/or PKS2 protein(s). The term “down-regulated” or “down-regulation” includes a decrease in expression of a gene or nucleic acid molecule of interest and/or the activity of an enzyme, e.g., a decrease in gene expression or enzymatic activity relative to the expression or activity in an otherwise identical gene or enzyme that has not been down-regulated.

Some aspects of the present disclosure include the partial, substantial, or complete deletion, silencing, inactivation, or down-regulation of expression of one or more particular polynucleotide sequences. The genes may be partially, substantially, or completely deleted, silenced, inactivated, or their expression may be down-regulated in order to affect the activity performed by the polypeptide they encode, such its enzymatic activity. Genes can be partially, substantially, or completely deleted, silenced, inactivated, or down-regulated by insertion of nucleic acid sequences that disrupt the function and/or expression of the gene, e.g., meganuclease engineering. The terms “eliminate,” “elimination,” and “knockout” can be used interchangeably with the terms “deletion,” “partial deletion,” “substantial deletion,” or “complete deletion” in accordance with the embodiments described herein.

A “recombinant” or “engineered” nucleic acid molecule is a nucleic acid molecule that has been altered through human manipulation, which can include robotic intervention. As non-limiting examples, a recombinant nucleic acid molecule includes any nucleic acid molecule that has been partially or fully synthesized or modified in vitro. Examples also include a nucleic acid molecule that includes conjoined nucleotide sequences that are not conjoined in nature. Examples may also include a nucleic acid molecule that has been engineered using molecular cloning techniques such that it lacks one or more nucleotides relative to the naturally occurring sequence. Examples may also include a nucleic acid molecule that has been manipulated using molecular cloning techniques such that it has one or more sequence changes or rearrangements with respect to the naturally occurring nucleic acid sequence.

The term “recombinant protein” as used herein refers to a protein produced by genetic engineering.

As used herein, “mutant” can refer to a plant cell, cell culture, specimen, seed, biomass, plant, plant part, or organism that has a mutation in at least one gene, e.g., pks1 and/or pks2, that is the result of some form of mutagenesis, e.g., endonuclease cleavage and double-stranded break repair. In some examples, the endonuclease may be encoded within a CRISPR construct. According to such examples, the endonuclease may comprise Cas9. “Mutant” may also refer to a recombinant cell, cell culture, specimen, seed, biomass, plant, or plant part that has altered structure, function, and/or expression of a gene as a result of genetic engineering targeting at least one gene involved in the production of one or more naturally occurring aloe toxins, such as aloin. Mutants can also refer to a recombinant cell, cell culture, specimen, seed, biomass, plant, or plant part that has attenuated expression of at least one gene necessary to produce at least one toxin. Such mutants may be generated by genetic disruption, targeted knockout, and/or nuclease activity, which may precede one or more natural or unnatural mechanisms of nucleic acid repair.

When applied to plant organisms, the term recombinant, engineered, or genetically modified refers to organisms that have been manipulated by the introduction of a heterologous or exogenous (e.g., non-native) recombinant nucleic acid sequence into the organism, non-limiting examples of which include gene knockouts, targeted mutations, and gene replacement, promoter replacement, deletion, or insertion, or transfer of a nucleic acid molecule, e.g., a transgene, synthetic gene, promoter, or other sequence into the organism. Recombinant or genetically engineered organisms can also be organisms into which constructs for gene “knock-down” have been introduced. Such constructs include, but are not limited to, one or more guide RNAs, transfer RNAs, RNA interference sequences, microRNAs, short hairpin RNAs, small interfering RNAs, antisense RNAs, and ribozyme constructs. Examples also include organisms having genomes altered by the activity of one or more Cas nucleases, meganucleases, or zinc finger nucleases. As used herein, “recombinant aloe” encompasses progeny or derivatives of the recombinant aloe of this disclosure. Because certain modifications may occur in succeeding generations due to either mutation or environmental influences, such progeny or derivatives may not, in fact, be identical to the parent cell, but are still included within the scope of the term “recombinant aloe.”

As mentioned above, the disclosed methods may involve one or more steps including, but not limited to: obtaining aloe plant cells from one or more plant sources, converting the obtained cells into stem cells, genetically modifying the stem cells to attenuate the expression of at least one gene necessary to synthesize at least one naturally-produced aloe toxin, generating toxin-free cell cultures therefrom, expanding the cell cultures to industrial volumes in bioreactors, and harvesting toxin-free or substantially toxin-free aloe from the cultured products. Embodiments may also involve growing toxin-free or substantially toxin-free aloe plants or plant parts using the modified cell cultures and harvesting toxin-free aloe and/or aloe seeds therefrom. Embodiments may involve each of the aforementioned steps, or may specifically exclude one or more steps. For example, certain embodiments may not involve one or more steps of converting source cells into stem cells, expanding the cell cultures in bioreactors, and/or growing genetically modified aloe plants or plant parts.

The specific, non-limiting embodiments described below involve suppressing aloin production via pks1 and/or pks2 attenuation, but such embodiments are described for illustrative purposes only. Additional embodiments may target one or more different aloe toxins and/or may reduce or eliminate toxin production by attenuating the expression of one or more different or additional genes and/or proteins involved in toxin biosynthesis. Such genes and/or proteins may be attenuated in addition to, or instead of, pks1, pks2, PKS1, and/or PKS2. The resulting mutants are collectively to as “toxin-free” or “substantially toxin-free” regardless of the specific toxin(s) targeted via the techniques described herein.

### Obtaining Aloe Source Cells

The mutant aloe cells generated according to the disclosed methods may be originally derived from wild-type source cells of various aloe species, including but not limited to: Aloe vera (or Aloe barbadensis Miller), Aloe arborescens, Aloe ferox, Aloe vaombe, Aloe decaryi, Aloe rubroviolacea, Aloe microstigma, Aloe broomii, Aloe aculeata, Aloe marlothii, Aloe cameronii, Aloe maculata, Aloe petricola, Aloe ciliaris, Aloe striata, Aloe hereroensis, Aloe perryi, Aloe polyphylla, Aloe brevifolia, Aloe aristata, Aloe plicatilis, Aloe capitala var. quartziticola, among others. The mutant aloe cells generated according to the disclosed methods may be originally derived from wild-type source cells of various aloe species originating from a variety of sources, non-limiting examples of which may include: greenhouses, farms, plant nurseries, stores that sell aloe plants, yards, the wild, among others. The source cells can be harvested directly from plant tissue or obtained in the form of established aloe cell lines. The particular tissue source of the source cells may include leaf, root, root hair, leaf trichome, stem, stalk, flower, pistil, stamen, petal, pollen, seed, embryo, cotyledon, meristem, among others. In some examples, the source of cells or the aloe plants themselves may be sterilized to remove or partially remove bacteria, fungi, or other contaminating organisms. Processes used to remove contaminating organisms may include application of 0-100% concentrations of bleach, hydrogen peroxide, chlorine gas, disinfecting solutions, among others. In some examples, the source of cells may be derived from plants that were grown in vitro, in plant tissue culture, in liquid, in a sterile environment, in a partially sterile environment, or from an unsterile environment.

The source cells may also be referred to as “control cells” or “control aloe,” each of which is a wild-type aloe cell or aloe from which the mutant aloe or mutant aloe cell is directly or indirectly derived. The control cells or control aloe can include aloe cells or aloe substantially identical to the manipulated, recombinant, or mutant cells, with the exception that the control cells do not have the genetic manipulation of the mutant aloe or aloe cells, i.e., the control cells do not have attenuated expression of pks1 and/or pks2, or attenuated production of PKS1, PKS2, and/or aloin.

### Stem Cell Generation

In some examples, the disclosed methods may involve converting at least a portion of the obtained aloe cells into stem cells. Embodiments of the stem cell conversion process may involve de-differentiating the source cells into multipotent, pluripotent or totipotent stem cells capable of being reprogrammed into specialized plant cells, including plant cells constituting the inner mucilaginous tissue of aloe leaves. The genetic engineering implemented to generate mutant aloe cell cultures according to the disclosed embodiments may thus be performed directly on control cells derived from aloe plants or on stem cells that have been, will be, or are currently undergoing a cellular differentiation process.

Controlled cellular differentiation may be performed by programming the stem cells according to a strict regimen that can involve applying specific growth factors, e.g., phytohormones, at specific time points under a particular set of environmental conditions. The end result may be a specialized aloe plant cell, for example an aloe plant cell constituting one or more layers of an aloe leaf, such as the substantially clear inner gel of the leaf. Programming the stem cells exclusively into a certain aloe plant cell type can result in selectively growing only the desired cells, i.e., not all the cells constituting an aloe plant or plant part. This approach may thus be used to significantly reduce waste and increase production rates. Successfully programmed cells may be expanded and maintained in cell cultures that can be treated in the manner necessary to ultimately generate the toxin-free aloe cells disclosed herein.

In some examples, the regeneration of mutant aloe, e.g., mutant aloe plants, can involve applying growth regulators such as about 0.1 to about 100 μg/mL of one or more auxin(s), e.g., 1-naphthaleneacetic acid (NAA), indoleacetic acid (IAA), indole-3-butyric acid (IBA), indole-3-propionic acid (IPA), 2-phenylacetic acid (PAA), or 4-chloroindole-3-acetic acid (4-CI-IAA). One or more of the aforementioned auxins can be applied alone or in combination with about 0.1 to about 100 μg/mL of one or more cytokinins, non-limiting examples of which can include 6-benzylaminopurine (BA), N6-benzylaminopurine riboside6-benzylaminopurine, 6-benzylaminopurine 9-(a-D-glucoside) (N6-Benzyladenine 9-Glucoside), and/or orphenylurea-type cytokinins, e.g., diphenylurea and thidiazuron (TDZ). Environmental factors including light and darkness levels, light period, and/or temperature can also be adjusted to stimulate regeneration alone or in combination with the plant growth regulators.

As noted above, alternative embodiments may exclude the stem cell conversion process. Such embodiments may involve infecting existing aloe plant cells with one or more exogenous nucleic acid constructs configured to attenuate the expression of pks1 and/or pks2, selecting the cells exhibiting pks1/2 attenuation and/or the absence of one or more toxins, such as aloin, and expanding the selected cells in culture.

### Gene Attenuation

A mutant aloe cell and/or mutant aloe cell culture having attenuated expression of one or more genes coding for PKS1 and/or PKS2 can be generated via one or more methods of genetic engineering disclosed herein. For example, embodiments may involve CRISPR-mediated editing of pks1, pks2, and/or at least one additional or different gene recognized by the inventors as being implicated in aloin biosynthesis. Additional means of attenuating pks1 and/or pks2, which may occur in addition to or instead of CRISPR-mediated editing, can involve homologous recombination, non-homologous end joining, RNA-guided nuclease activity, RNAi constructs, ribozyme constructs, transcription activator-like effector nuclease activity (“TALENS”), zinc finger nuclease activity (“ZFNs”), meganuclease activity, or combinations thereof.

As used herein, “attenuating expression of a pks1 and/or pks2 gene” can mean reducing, altering, or eliminating wild-type expression of the gene(s) in any manner that alters or reduces production of the fully functional PKS1 and/or PKS2 protein, which may be comprised of an amino acid sequence having at least 55%, 60%, 70%, 75%, 80%, 85%, 90%, 95%, 98%, or 99% identity to SEQ ID NO: 1 and SEQ ID NO:2, respectively. Mutant aloe engineered to have attenuated expression of pks1, pks2, or both, can have a pks1 and/or pks2 gene that includes at least one mutation, deletion, or insertion that reduces or abolishes expression of the gene such that a fully functional or wild-type pks1 and/or pks2 gene is not produced or is produced in significantly lower amounts than in wild-type aloe.

In embodiments involving CRISPR-mediated editing, the pks1 and/or pks2 gene may be disrupted by the DNA cleavage activity of a Cas protein (e.g., Streptococcus pyogenes Cas9), which is a DNA endonuclease configured to create double-stranded DNA breaks. The particular endonuclease may vary, and a given vector encoding the CRISPR components may include a Cas9, Cas12a, SaCas9, NmeCas9, StCas9, TdCas9, CjCas9, MAD7, or Cpf1 expression cassette that, in addition to an endonuclease gene derived from Streptococcus pyogenes, Acidaminococcus, Francisella novicida, Staphylococcus aureus, Neisseria meningitidis, Streptococcus thermophilus, Treponema denticola, Campylobacter jejuni, and/or Eubacterium rectale, can include sequences encoding an N-terminal FLAG tag and nuclear localization signal.

In addition to the endonuclease polypeptide or complex, the CRISPR system includes at least one synthetic guide RNA (“gRNA”) configured to interact with a genomic target site in aloe cells by complementarity with a specific target site sequence. A gRNA sequence that is complementary to at least a portion of a genomic target site may be referred to as the “gRNA spacer sequence,” and may be about 20 to about 23 nucleotides in length. The target sequence located by the spacer sequence may be located within or adjacent to pks1 and/or pks2, for example within the coding region or a transcriptional regulatory sequence. Specific examples disclosed herein include a gRNA spacer sequence configured to target at least a portion of a naturally-occurring gene encoding the PSK1 or PKS2 protein, such as pks1 and/or pks2. In some examples, a single CRISPR construct may include multiple gRNAs to enable multiplexed sequence targeting, which may improve the effectiveness of genetic attenuation.

The CRISPR RNA sequences disclosed herein may be plasmid-expressed DNA sequences transcribed into RNA after introduction of the plasmid(s) into the targeted aloe cells via the endogenous RNA polymerases present within the cells. Accordingly, a gRNA spacer sequence configured to target at least a portion of the pks1 gene may initially have a DNA sequence identical or substantially identical to SEQ ID NO:3, SEQ ID NO:4, SEQ ID NO:5, or SEQ ID NO:6. A gRNA spacer sequence configured to target at least a portion of the pks2 gene may initially have a DNA sequence identical or substantially identical to SEQ ID NO:7, SEQ ID NO:8, SEQ ID NO:9, or SEQ ID NO:10. Substantially identical sequences may comprise a nucleotide sequence having at least 55%, 60%, 70%, 75%, 80%, 85%, 90%, 95%, 98%, or 99% identity to any of the aforementioned SEQ ID NOS: 3-10. These gRNA spacer sequences can be inserted into a plasmid separately or together. Guide RNAs effective for targeting pks1 and/or pks2 may not be limited to SEQ ID NOS: 3-10, and may also include additional sequences identified computationally. Additional, non-limiting examples of gRNA sequences (provided as plasmid-expressed DNA sequences) targeting pks1 and pks2 may be identical or substantially identical to SEQ ID NOS: 24-567 and SEQ ID NOS: 568-1079, respectively. Design of the disclosed gRNAs was informed in part by the conserved protein domains present in PKS1 and PKS2. Guide RNAs targeting one or more of the underlying genetic code for these domains may be most effective in attenuating PKS1 and/or PKS2 assembly, activity, intensity, and/or function.

In addition to the gRNA spacer sequence complementary to a target sequence, a full gRNA cassette can also include a scaffold sequence and/or a transfer RNA (“tRNA”) sequence, which may flank a spacer sequence at its 5′ and 3′ ends. The scaffold sequence is configured to bind with a binding site on the endonuclease protein, e.g., Cas protein, thereby ensuring delivery of the protein to the target site, guided by a gRNA spacer sequence. In this manner, a functional endonuclease can attenuate a gene encompassing or in proximity to the target site by creating a targeted double-stranded break, which in some examples may be repaired by error-prone endogenous repair enzymes.

The tRNA sequences can be included to express multiple gRNA spacer sequences in a single nucleic acid transcript, which can enable simultaneous targeting and attenuation of multiple independent loci. Transfer RNA is an RNA molecule that contributes to amino acid recruitment and assembly using the underlying mRNA instructions. The tRNA sequence may be cleaved by endogenous RNA-processing enzymes, such that if multiple gRNA spacer sequences are flanked by an individual tRNA, then multiple functional gRNAs are released within the cell for target hybridization and cleavage.

With the tRNA and gRNA scaffold sequences flanking each gRNA spacer, the full gRNA cassette sequence for spacer SEQ ID NOS: 3-10 may comprise a sequence identical or substantially identical to SEQ ID NO: 11, SEQ ID NO: 12, SEQ ID NO: 13, SEQ ID NO: 14, SEQ ID NO: 15, SEQ ID NO: 16, SEQ ID NO: 17, and SEQ ID NO: 18, respectively, which are again provided as DNA sequences expressed within the target cells by endogenous RNA polymerase. Substantially identical sequences may comprise a nucleotide sequence having at least 55%, 60%, 70%, 75%, 80%, 85%, 90%, 95%, 98%, or 99% identity to any of the aforementioned SEQ ID NOS: 11-18.

A single promoter can be used to express all gRNA spacer sequences, scaffolds and tRNAs. The promoter may be constitutive, e.g., U6, or it may be expressed at specific times and/or in specific locations. The U6 promoter can be identical or substantially identical to SEQ ID NO: 19. Substantially identical sequences may comprise a nucleotide sequence having at least 55%, 60%, 70%, 75%, 80%, 85%, 90%, 95%, 98%, or 99% identity to SEQ ID NO: 19. Alternatively, each gRNA cassette (comprised of a spacer, scaffold and/or tRNA sequence) included in an exogenous expression vector can be transcribed by an individual promoter.

Aloe mutants can be selected by identifying one or more characteristics of successfully mutated cells, e.g., fluorescence and/or antibiotic resistance. Mutants can also be confirmed via one or more techniques including PCR amplification, quantitative PCR, and/or DNA sequencing. In addition, microtiter or otherwise high-throughput phenotyping assays could be employed including, but not limited to: spectroscopy, thin layer chromatography, and enzyme activity assays.

Before the DNA-editing machinery can target and attenuate one or more target sites, the nucleic acid construct encoding the machinery, e.g., a circular DNA plasmid hereinafter referred to as the “attenuation construct,” must be inserted into the target cells, which may be in culture or constituting fully formed plant tissue. As mentioned above, the target cells can include wild-type aloe cells or reprogrammed stem cells maintained in a cell culture. In some embodiments, the attenuation construct and/or complementary ribonucleoprotein (RNP) may be inserted into the target cells by particle bombardment (i.e., biolistics, gene gun), bacterial transformation (e.g., Agrobacterium-mediated transformation or Rhizobium-mediated transformation), or protoplast transfection or transformation. Non-limiting examples of additional bacterial cells transformed in accordance with the disclosed embodiments can include cells derived from Agrobacteria tumefaciens, Ensifer adhaerens, Ochrobactrum haywardense, Rhizobium etli, Sinorhizobium meliloti, Mesorhizobium loti, Rhizobium sp. strain NGR234, or a combination thereof. Examples may also involve delivering an attenuation construct to target cells using carbon nanotubes, silicon carbide whiskers, direct injection, or a combination thereof. In some embodiments, incorporating the attenuation construct into the cultured cells can be achieved according to a two-step process. Step one can involve transforming bacterial host cells with the attenuation construct, which is then copied via the bacterial cell's machinery. Step two can involve infecting the transformed bacterial cells into the target cells, where the attenuation constructs are transcribed and translated.

In some embodiments, transformation can be achieved by electroporating competent bacterial cells, such as Agrobacterium tumefaciens cells (hereinafter “Agrobacterium”), which are naturally-occurring soil bacteria able to introduce exogenous nucleic acids into the genome of the plant cells via endogenous Transfer DNA (“T-DNA”). The Agrobacterium cells may be transformed according to various techniques, which can include heat-shock and/or electroporation.

In one example, an overnight culture of competent Agrobacterium cells can be grown in the presence of one or more antibiotics. When the bacterial cell culture reaches log growth, e.g., an OD600 reading between about 0.6 and 1.0, the cells can be spun to form a concentrated pellet. After washing one or more times, the pellet can be resuspended in deionized water, and a portion of the resuspended bacteria, e.g., about 80 μL, can be transferred to a separate microcentrifuge tube, to which the attenuation construct can also be added. The amount of attenuation construct added to the bacterial suspension may vary, ranging from about 25 to about 250 ng in various embodiments.

The DNA-inoculated bacterial suspension can then be transferred to a cuvette, e.g., 2 mm cuvette, and electroporated at about 2.5 kv, for example. After electroporation, about 900 μL of Super Optimal broth with Catabolite repression (“S.O.C.”) medium can be added to the transformed bacterial culture, which may then be incubated for about 3 or more hours at about 28° C. The resulting culture can then be centrifuged and the supernatant removed. After resuspension, the transformed bacteria can be spread onto selective media, e.g., agar infused with one or more antibiotics encoded within the attenuation construct. Encoded antibiotics may include kanamycin and/or spectinomycin, for example. DNA from the transformed Agrobacterium may be extracted and sequenced to confirm successful transformation.

After transforming the bacterial cells with the attenuation construct, the construct can be infected into the target plant cells. In some examples, this process may begin after growing the transformed cells on a culture medium supplemented with one or more antibiotics, e.g., agar infused with spectinomycin and/or kanamycin, for about two days at about 28° C. A single bacterial culture can then be chosen and inoculated into a liquid culture medium supplemented with at least one antibiotic and agitated overnight at about 28° C. on a shaker set at about 180 rpm, for example. Once the OD600 reading of the culture reaches at least about 0.8, the liquid culture can be pelleted, the supernatant discarded, and the bacteria resuspended in an “infection medium” having a pH of about 5.7. The resulting resuspension can be supplemented with acetosyringone and the cell density adjusted to about 0.6 to about 0.8.

Pursuant to the target cell infection step, an aloe plant cell culture can be inoculated into a test tube or vial, e.g., a 50 mL conical tube. The cell culture can be centrifuged and resuspended with about 5 mL of the transformed Agrobacterium and infection medium. The mixture can then be vortexed, pelleted and resuspended in a co-cultivation medium. The supernatant can then be removed and the remaining Agrobacterium resuspended. The resulting cell culture can be pelleted at least once thereafter, and after a relatively prolonged period, e.g., about two to three weeks, the cell culture can be resuspended in a new medium, e.g., a medium containing hygromycin. The culture can be observed microscopically for a period of about six to about eight weeks. The culture can be screened with a cell counter and diluted as necessary.

After incubating the cell culture samples until antibiotic-resistant colonies appear, the colonies can be selected for DNA extraction and amplification via PCR to confirm successful attenuation, e.g., knockout, of pks1 and/or pks2. To confirm a successful pks1 knockout, a forward primer used for PCR may have a sequence identical or substantially identical to SEQ ID NO: 20, and a reverse primer may have a sequence identical or substantially identical to SEQ ID NO: 21. To confirm a successful pks2 knockout, a forward primer used for PCR may have a sequence identical or substantially identical to SEQ ID NO: 22, and a reverse primer may have a sequence identical or substantially identical to SEQ ID NO: 23. Substantially identical sequences may comprise a nucleotide sequence having at least 55%, 60%, 70%, 75%, 80%, 85%, 90%, 95%, 98%, or 99% identity to any of the aforementioned SEQ ID NOS: 20-23. In addition or alternatively, knockout confirmation may be achieved by PCR-based amplification of one or more sequences containing at least a portion of an attenuation construct. DNA sequencing of at least a portion of the targeted attenuation site may also be performed to confirm successful editing.

Attenuation of pks1 and/or pks2 expression can be defined as expression reduced by at least 50%, at least 55%, at least 60%, at least 65%, at least 70%, at least 75%, at least 80%, at least 85%, at least 90%, at least 95%, or greater than 95% relative to wild-type expression levels. The attenuated expression may be reduced to undetectable levels in some embodiments. As noted above, attenuation can also encompass wild-type expression of a mutated pks1/pks2 gene. According to such embodiments, the structure, function, and/or production level of the resulting PKS1/PKS2 protein may be disrupted.

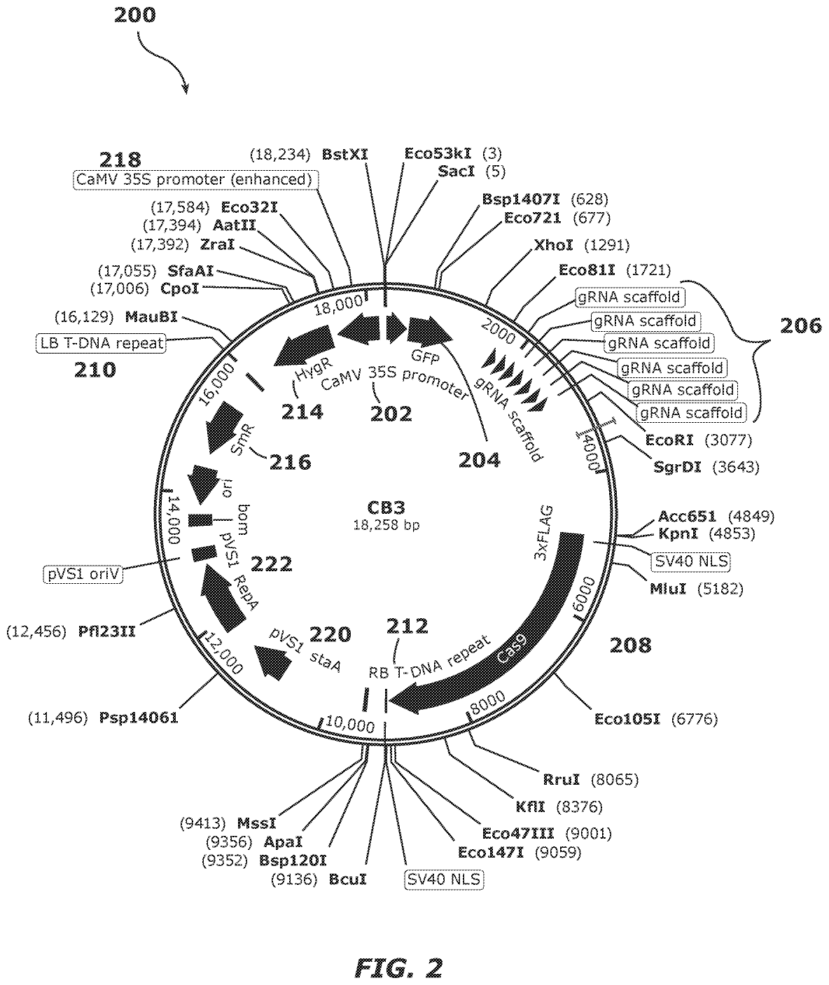

An example of an ˜18,000-bp construct used to knockdown or otherwise attenuate production of the PKS1 and/or PKS2 proteins via CRISPR-mediated editing of the pks1 and/or pks2 genes in aloe host cells is depicted in FIG. 2. The plasmid vector 200 shown comprises a backbone template construct modified to include sequences specific to the processes implemented herein. As shown, the construct may comprise a binary plasmid 200 containing numerous sequences necessary for successful bacterial transformation, target cell infection, and attenuation construct expression. In particular, the plasmid 200 includes at least one plant-specific promoter 202 configured to drive expression of the CRISPR components in the infected plant cells. In the example shown, the plant-specific promoter 202 comprises a sequence derived from the cauliflower mosaic virus, called CaMV 35S. A sequence encoding a green fluorescent protein (“GFP”) 204 is also included downstream of the promoter 202. Expression of the GFP construct 204 in the targeted plant cells enables fluorescent selection of the cells expressing the attenuation construct.

The plasmid 200 also includes a gRNA cassette comprised of multiple gRNAs 206 (provided as DNA for later transcription) and a Cas9 coding region 208. In this particular embodiment, the gRNA cassette includes DNA sequences corresponding to six gRNAs, each of which may include a gRNA spacer sequence and Cas9 scaffold, along with a tRNA sequence for multiplexed expression. Together, the gRNAs may target sequences within or overlapping with pks1 and/or pks2, such that one or both genes are effectively attenuated via targeted Cas9 cleavage. The gRNA cassette, along with one or more additional components, can be cloned into the plasmid 200 using various techniques, including various restriction enzyme-based methods, and/or one or more commercially available cloning systems, such as Gateway® cloning or Golden Gate assembly.

To ensure successful transfer of the attenuation construct, including the gRNA cassette and GFP sequences, into the targeted plant cells, the plasmid 200 also includes a left-border T-DNA repeat (“LB T-DNA”) 210 and a right-border T-DNA repeat (“RB T-DNA”) 212, each of which can comprise approximately 25 basepairs. The region flanked by these sequences is transferred to the targeted plant cells by the bacterial cells transformed with the plasmid 200. After successful plant cell infection, the transferred gRNA cassette may be integrated within the genome of the infected plant cells, where the cassette is transcribed and translated using plant host machinery.

Multiple antibiotic-resistance genes are also included to facilitate antibiotic selection of bacterial cells successfully transformed with the plasmid 200 and plant cells successfully infected with the plasmid 200. The example shown includes a hygromycin-resistance gene 214 and a streptomycin-resistance gene 216, both of which are expressed by an enhanced CaMV 35S promoter 218.

A pVS1 StaA sequence 220 is included to facilitate stable plasmid segregation in the transformed bacterial cells, and a pVS1 RepA sequence 222 is included to facilitate consistent plasmid replication within the bacterial cells.

### Cell Culture

Mutant aloe plant cells can be selected for continued propagation using a variety of techniques. Such techniques may include antibiotic selection and/or fluorescence-based selection. In some examples, selection can be achieved at least in part by utilizing the expression of one or more protein products encoded by the plasmid 200 transformed into bacterial cells and infected within the host plant cells. For example, mutant aloe cells can be selected via their resistance to hygromycin and/or streptomycin, among other antibiotics. Mutant cells can also be selected by the expression of a fluorescent construct included within the attenuation construct, such as a construct encoding GFP or dsRed. The natural fluorescence of cells producing aloin may also facilitate the mutant selection process. High-throughput robotics can be employed to aid any of the aforementioned processes by continuously selecting the cells expressing one or more selectable trait(s).

After selecting aloe plant cells exhibiting successful toxin suppression, methods can involve culturing the mutant aloe cells according to a customized culturing program. The culturing program may involve growing the cells in a suitable medium under controlled environmental conditions designed to foster cell growth, which may be measured by increases in cell size, cellular contents, and/or cellular activity. Vessels for suspension growth that can be used include test tubes, jars, flasks (both baffled and unbaffled), bioreactors, fermenters, buckets, tanks, drums, pipes, or bags. Cells may or may not require to be maintained in suspension, in which case mixing can be achieved by using either or in combination magnetic stir bars, paddles, pumps, shaking, or gas sparging. In some embodiments, cells can also be grown on adherent support systems such as roller bottles, multi-layer cell culture trays, foam, beads, fibers, or microcarriers. In some embodiments, gas exchange may be provided to cultures including 0.01-100% air, 0.1-100% pure nitrogen, 0.1-100% carbon dioxide, 0.1-100% pure oxygen, or any combination thereof. The use of gas permeable materials including but not limited to polymers like polyethylene (PE) film or fibers and foam or natural fiber plugs may be used as an alternative to active gas exchange, when gas exchange is actively introduced, the gas flow rate provided to the cells can be from 1-100 L/min. In the case of active addition of gases, it may be filtered to 0.2 μM or less to maintain sterility. Cell growth may also be measured by cell propagation, which may be measured by increases in total cell count, for example through microscopy or flow cytometry. For ease of illustration, the combination of cell growth and propagation may be referred to as “proliferation” herein.

The particular nutrients and buffers included in the cell culture medium may have a significant impact on cell culture health and growth. Accordingly, the cell culture medium used to grow the toxin-free cells described herein may include novel combinations and amounts of one or more growth-promoting components, such as sugars, nitrates, phosphates, growth regulators, and/or carbon sources. The cell culture medium may also have a specific pH or pH range.

The particular cell culture conditions may also be unique to the methods described herein for enhancing cell growth, development, and metabolite production. Accordingly, the conditions used to grow the toxin-free mutant cells may include novel temperatures, humidity levels, carbon dioxide levels, aeration levels, perturbation conditions (e.g., shaking), and/or light application.

The cell culture conditions can be tailored to photoautotrophic plants. Aloe cell cultures grown photoautotrophically can be grown on a culture medium in which inorganic carbon is substantially the sole source of carbon. For instance, in a culture in which inorganic carbon is substantially the sole source of carbon, any organic carbon molecule or organic carbon compound that may be provided in the culture medium either cannot be taken up and/or metabolized by the cell for energy and/or is not present in an amount sufficient to provide sustainable energy for cell culture proliferation. Cells grown photoautotrophically can be grown under constant light or a diel cycle, for example a diel cycle in which the light period can be, for example, at least four hours, about five hours, about six hours, about seven hours, about eight hours, about nine hours, about ten hours, about 12 hours, about 14 hours, about 16 hours, more than 16 hours, or any length of time therebetween.

At least initially, the cell cultures can be maintained and/or expanded in petri dishes, shake flasks, test tubes, growth plates, vials, and/or microtiter dishes. Such devices may be placed within growth chambers programmed to maintain the cell culture conditions disclosed herein.

By implementing the cell culture conditions described herein, the cultures may expand at a doubling rate of less than about two days, about two days, about three days, about four days, about five days, about six days, more than six days, or any length of time therebetween. Specific embodiments may exhibit a doubling rate of about two to three days. Such proliferation rates may be approximately similar to the proliferation rates achieved in wild-type aloe cell cultures, or the proliferation rates may be accelerated relative to wild-type and/or genetically modified cell cultures.

The cultured cells may be expanded in any suitable vessel, including flasks or bioreactors, where the mutant aloe may be exposed to artificial or natural light in the presence of specialized media without the use of soil. The culture comprising the mutant aloe may be cultured on a light/dark cycle that may be a natural or programmed light/dark cycle. Embodiments may involve culturing the toxin-free cells in one or more bioreactors equipped with an artificial light source and/or having exterior walls configured to allow the passage of natural light sufficient to foster cell culture proliferation. Each bioreactor can supply a source of inorganic carbon to the growing cell cultures. Non-limiting examples of inorganic carbon can include carbon dioxide, bicarbonate, and/or carbonate salts, which may be provided by air, CO₂-enriched air, flue gas, or combinations thereof.

As used herein, the term “bioreactor” refers to an apparatus configured to support and expand a population of mutant aloe plant cells by maintaining an internal environment conducive to cell viability and growth. The apparatus can comprise a vessel, tank or chamber configured to stir, rock or otherwise mix the cultured cells contained therein. Specific embodiments may also include an apparatus configured to air-wheel mix, bubble mix and/or orbitally-shake a population of cultured plant cells. Example stirred-tank bioreactors can be equipped with one or more support tanks containing impellers for mixing and spargers for culture gassing. The material on which the cultured cells are attached in stir-mixed apparatuses can comprise substantially rigid or flexible material, either of which may comprise one or more plastics. Apparatuses configured to rock cultured cells can comprise a generally flexible chamber resembling a plastic pillow or pouch propped upon a tray configured to move back and forth for continual mixing action and gas transfer.

After a period of expansion within one or more bioreactors, the mutant aloe cells may be harvested for toxin-free aloe bioproducts. The term “bioproduct” used herein refers to a product comprising or derived from the mutant cells described in accordance with the disclosed embodiments, i.e., cells lacking or substantially lacking one or more toxins, e.g., aloin. Such products can be in dry form, e.g., powder, wet form, e.g., liquid, and/or mucilaginous form e.g., gel. The products can comprise various amounts of a molecule or class of molecules. Non-limiting examples of such products include a bioproduct comprised of at least one polysaccharide, a glucomannan, a galactomannan, a nucleoside diphosphate sugar, a beta hydroxy acid, a pentacyclic diterpenoid, a heptaketide, an aromatic polyketide, a cinnamate ester, a chromone, an anthraquinone, a dihydroxyanthraquinone, an anthrone, an octaketide, a benzoisochromanequinone, a C-glucosylanthrone, a coenzyme, an anthracene, a chalconoid, a lipid, an amino acid, a protein, a carbohydrate, an aldopentose, a sterol, a steroid, a mineral, a medium chain fatty acid, a pectin, and/or a triglyceride. Particular embodiments of the disclosed bioproducts may lack or substantially lack one or more naturally produced toxins, such as aloin, and may include one or more polysaccharides, minerals, sugars, proteins, lipids, fatty acids, esters, and/or phenolic compounds. Wet or mucilaginous bioproducts may produce any or all of the aforementioned components in a water-based suspension. In embodiments, the harvested bioproduct(s) can comprise or be incorporated into various end products, such as products for consumer use, non-limiting examples of which can include aloe-based gels, creams, lotions, soaps, sunscreens, sprays, haircare products, jellies, moisturizers, cleansers, toners, skin treatment compositions, cosmetics, mouthwashes, toothpastes, edible foods and/or liquids, e.g., water or juice.

The toxin-free cell cultures can also be recovered in whole or in part. In some examples, one or more polysaccharides, proteins, lipids, and/or biomasses may be selectively extracted from the whole-cell cultures. Biomasses can be harvested by centrifugation and/or filtering, which may be followed by one or more processing steps, such as drying, concentrating and/or grinding to produce a purified, toxin-free aloe powder. Toxin-free aloe powder may be reconstituted by adding one or more liquids or gels, for instance. According to some implementations, at least 1 gram of toxin-free aloe powder can be produced per liter of bioreactor specimen per day. Toxin-free aloe bioproducts can also be filtered and/or sterilized to remove any impurities. Whether in liquid, mucilaginous, or dry form, the toxin-free aloe bioproducts may be pure or substantially pure, meaning the products lack or substantially lack components not naturally present in harvested aloe. In addition or alternatively, the bioproducts may be supplemented with one or more additives or preservatives to enhance or stabilize the desired aloe traits. The final product may also be regenerated toxin-free aloe plants or plant products, e.g., plant parts or seeds, having attenuated pks1/2 expression.

The disclosed cell cultures may be expanded into industrial-scale production systems configured to generate large volumes of toxin-free plant products. With increased scale, a 25,000 liter reactor can produce a toxin-free aloe at the equivalent rate of a 100-hectare farm. Cultures can be scaled-up to larger reactors, for example reactors having a volume of 74 liters, 100 liters, 125 liters, 150 liters, 175 liters, 200 liters, 225 liters, 250 liters, 275 liters, 300 liters, greater than 300 liters, or any volume therebetween. This can be scaled up to about a 75,000 liter bioreactor.

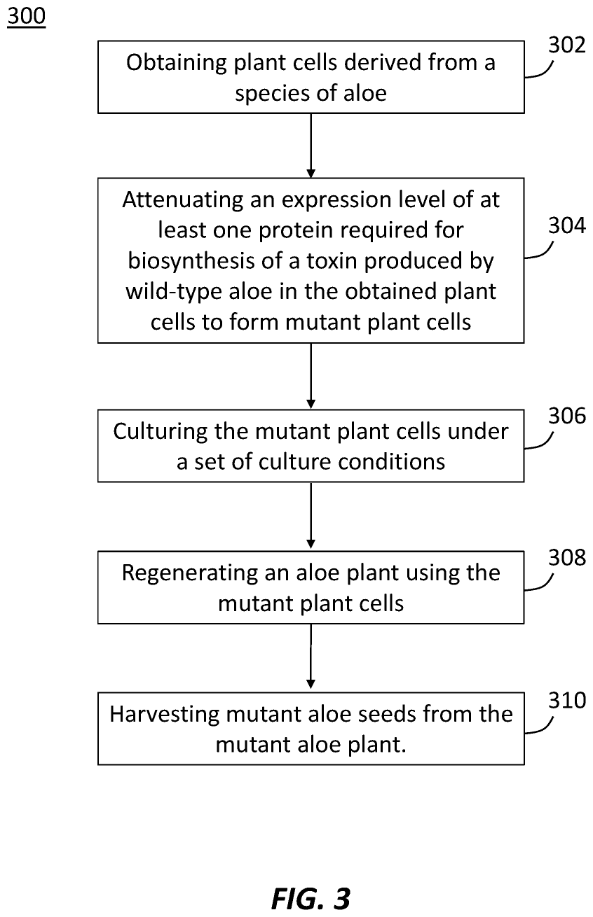

FIG. 3 is a flow diagram depicting another method 300 implemented in accordance with embodiments described herein. As shown, the method 300 may involve, at step 302, “obtaining plant cells derived from a species of aloe.” At step 304, the method 300 may involve “attenuating an expression level of at least one protein (e.g., PKS1 and/or PKS2) required for biosynthesis of a toxin produced by wild-type aloe in the obtained plant cells to form mutant plant cells.” At step 306, the method 300 may involve “culturing the mutant plant cells under a set of culture conditions.” As noted above, methods may also involve optionally converting the obtained plant cells into plant stem cells prior to attenuating the expression level of at least one protein required for biosynthesis of the toxin. At step 308, the method 300 may additionally involve “regenerating an aloe plant using the mutant plant cells.” At step 310, the method 300 may further involve “harvesting mutant aloe seeds from the mutant aloe plant.”

As used herein, the term “about” modifying, for example, the quantity of a component in a composition, concentration, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or components used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities.

Similarly, it should be appreciated that in the foregoing description of example embodiments, various features are sometimes grouped together in a single embodiment for the purpose of streamlining the disclosure and aiding in the understanding of one or more of the various aspects. These methods of disclosure, however, are not to be interpreted as reflecting an intention that the claims require more features than are expressly recited in each claim. Rather, as the following claims reflect, inventive aspects lie in less than all features of a single foregoing disclosed embodiment, and each embodiment described herein may contain more than one inventive feature.

Although the present disclosure provides references to preferred embodiments, persons skilled in the art will recognize that changes may be made in form and detail without departing from the spirit and scope of the invention.

What is claimed is:

1. A method of producing a mutant aloe, the method comprising:
obtaining plant cells derived from a species of aloe;
attenuating an expression level of at least one protein required for biosynthesis of a toxin produced by wild-type aloe in the obtained plant cells to form mutant plant cells, wherein the at least one protein comprises polyketide synthase 1 (PKS1) and/or polyketide synthase 2 (PKS2); and
culturing the mutant plant cells under a set of culture conditions.

2. The method of claim 1, further comprising converting the obtained plant cells into plant stem cells prior to attenuating the expression level of at least one protein required for biosynthesis of the toxin.

3. The method of claim 1, further comprising growing the cultured mutant plant cells in a bioreactor and harvesting an aloe bioproduct from the cultured mutant plant cells.

4. The method of claim 1, wherein the toxin comprises aloin.

5. The method of claim 1, further comprising regenerating an aloe plant using the mutant plant cells.

6. The method of claim 1, further comprising selecting the mutant plant cells before culturing the mutant plant cells under a set of culture conditions by screening the mutant plant cells for fluorescence and/or applying one or more antibiotics to the mutant plant cells.

7. The method of claim 1, wherein attenuating an expression level of at least one protein required for biosynthesis of a toxin comprises disrupting an enzymatic activity of the at least one protein.

8. The method of claim 1, wherein attenuating the expression level of at least one protein comprises disrupting expression of the pks1 and/or pks2 gene.

9. The method of claim 8, wherein disrupting expression of the pks1 and/or pks2 gene comprises:
transforming bacterial cells with an endonuclease configured to cleave pks1 and/or pks2; and
infecting the obtained plant cells with the transformed bacterial cells.

10. The method of claim 9, wherein the endonuclease is encoded in a CRISPR construct comprising a guide RNA sequence having at least 55% identity to SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

11. The method of claim 9, wherein disrupting expression of the pks1 and/or pks2 gene comprises delivering an endonuclease and/or ribonucleoprotein targeting pks1 and/or pks2 to the obtained plant cells via one or more of: particle bombardment, carbon nanotubes, silicon carbide whiskers, or direct injection.

12. A mutant aloe that has attenuated expression of at least one polyketide synthase (pks) gene.

13. The mutant aloe of claim 12, wherein the at least one pks gene comprises pks1 and/or pks2.

14. The mutant aloe of claim 13, wherein an amount of mRNA transcribed by pks1 and/or pks2 is less than 10% of an amount of mRNA transcribed by pks1 and/or pks2 in wild-type aloe.

15. The mutant aloe of claim 12, wherein the mutant aloe comprises a mutant aloe cell or cell culture.

16. The mutant aloe of claim 12, wherein the mutant aloe comprises mutant Aloe vera.

17. The mutant aloe of claim 12, wherein the mutant aloe comprises a mutant aloe bioproduct comprising or included in one or more of: an aloe-based gel, cream, lotion, soap, sunscreen, spray, haircare product, jelly, moisturizer, cleanser, toner, skin treatment composition, cosmetic, mouthwash, toothpaste, edible food or liquid.

18. The mutant aloe of claim 12, wherein the mutant aloe exhibits an absence of one or more toxins produced by wild-type aloe.

19. The mutant aloe of claim 18, wherein the one or more toxins comprise aloin.

20. The mutant aloe of claim 19, wherein an amount of aloin produced in the mutant aloe is less than 10% of an amount of aloin produced in wild-type aloe.


SEQUENCE LISTING (1079 sequences)

SEQ ID NO: 1 - protein, 403 aa, Aloe sp.
MGSLSDSTPLMKDVQGIRKAQKADGTATVMAIGTAHPPHIISQDSYADFYFRVTNSEHKVELKKKFDRICKKTMIGKRYFNFDEEFLKKYPNITSFDKPSLNDRHDICIPGVPALGAEAAVKAIEEWGRPKSEITHLVFCTSGGVDMPSADFQCAKLLGLRTNVNKYCIYMQGCYAGGTVMRYAKDLAENNRGARVLMVCAELTIIALRGPNDSHIDNAIGNSLFGDGAAALIVGSDPIIGVEKPMFEIVCAKQTVIPNSEDVIHLHLRESGLMFYMTKDSAATISNNIEACLVDVFKSVGMTPPEDWNSLFWIPHPGGRAILDQVEAKLKLRPEKFSATRTVLWDYGNMISACVLFILDEMRRKSAAEGLETYGEGLEWGVLLGFGPGMTIETILLHSLPPV

SEQ ID NO: 2 - protein, 403 aa, Aloe sp.
MGSLSNYSPVMEDVQAIRKAQKADGTATVMAIGTAHPPHIFPQDTYADFYFRATNSEHKVELKKKFDRICKKTMIGKRYFNYDEEFLKKFPNITSFDEPSLNDRQDICVPGVPALGAEAAVKAIAEWGRPKSEITHLVFCTSCGVDMPSADFQCAKLLGLRANVNKYCVYMQGCYAGGTVMRYAKDLAENNRGARVLVVCAELTIIGLRGPNESHLDNAIGNSLFGDGAAALIVGSDPIIGVEKPMFEIVCAKQTVIPNSEDVIHLHMREAGLMFYMSKDSPETISNNVEACLVDVFKSVGMTPPEDWNSLFWIPHPGGRAILDQVESRLKLRPEKFGATRTVLWDCGNMVSACVLYILDEMRRKSAADGLATYGEGLEWGVLLGFGPGMTVETILLHSLPTV

SEQ ID NO: 3 - DNA, 23 bp, Artificial Sequence
  Description of Artificial Sequence Synthetic oligonucleotide
catttcgcaggacagctacgcgg

SEQ ID NO: 4 - DNA, 23 bp, Artificial Sequence
  Description of Artificial Sequence Synthetic oligonucleotide
tcagacccgactccctcaggtgg

SEQ ID NO: 5 - DNA, 23 bp, Artificial Sequence
  Description of Artificial Sequence Synthetic oligonucleotide
gcatgacatttgcattcctgggg

SEQ ID NO: 6 - DNA, 23 bp, Artificial Sequence
  Description of Artificial Sequence Synthetic oligonucleotide
gagtcgcactaaacttctcgggg

SEQ ID NO: 7 - DNA, 23 bp, Artificial Sequence
  Description of Artificial Sequence Synthetic oligonucleotide
atgcagggatgctacgctggcgg

SEQ ID NO: 8 - DNA, 23 bp, Artificial Sequence
  Description of Artificial Sequence Synthetic oligonucleotide
aggcatgtcgacaccgcaggagg

SEQ ID NO: 9 - DNA, 23 bp, Artificial Sequence
  Description of Artificial Sequence Synthetic oligonucleotide
ccaggatatttgcgtccctgggg

SEQ ID NO: 10 - DNA, 23 bp, Artificial Sequence
  Description of Artificial Sequence Synthetic oligonucleotide
ttctggatccctcaccccggcgg

SEQ ID NO: 11 - DNA, 176 bp, Artificial Sequence
  Description of Artificial Sequence Synthetic polynucleotide
aacaaagcaccagtggtctagtggtagaatagtaccctgccacggtacagacccgggttcgattcccggctggtgcacatttcgcaggacagctacgcgggttttagagctagaaatagcaagttaaaataaggctagtccgttatcaacttgaaaaagtggcaccgagtcggtgc

SEQ ID NO: 12 - DNA, 176 bp, Artificial Sequence
  Description of Artificial Sequence Synthetic polynucleotide
aacaaagcaccagtggtctagtggtagaatagtaccctgccacggtacagacccgggttcgattcccggctggtgcatcagacccgactccctcaggtgggttttagagctagaaatagcaagttaaaataaggctagtccgttatcaacttgaaaaagtggcaccgagtcggtgc

SEQ ID NO: 13 - DNA, 176 bp, Artificial Sequence
  Description of Artificial Sequence Synthetic polynucleotide
aacaaagcaccagtggtctagtggtagaatagtaccctgccacggtacagacccgggttcgattcccggctggtgcagcatgacatttgcattcctgggggttttagagctagaaatagcaagttaaaataaggctagtccgttatcaacttgaaaaagtggcaccgagtcggtgc

SEQ ID NO: 14 - DNA, 176 bp, Artificial Sequence
  Description of Artificial Sequence Synthetic polynucleotide
aacaaagcaccagtggtctagtggtagaatagtaccctgccacggtacagacccgggttcgattcccggctggtgcagagtcgcactaaacttctcgggggttttagagctagaaatagcaagttaaaataaggctagtccgttatcaacttgaaaaagtggcaccgagtcggtgc

SEQ ID NO: 15 - DNA, 176 bp, Artificial Sequence
  Description of Artificial Sequence Synthetic polynucleotide
aacaaagcaccagtggtctagtggtagaatagtaccctgccacggtacagacccgggttcgattcccggctggtgcaatgcagggatgctacgctggcgggttttagagctagaaatagcaagttaaaataaggctagtccgttatcaacttgaaaaagtggcaccgagtcggtgc

SEQ ID NO: 16 - DNA, 176 bp, Artificial Sequence
  Description of Artificial Sequence Synthetic polynucleotide
aacaaagcaccagtggtctagtggtagaatagtaccctgccacggtacagacccgggttcgattcccggctggtgcaaggcatgtcgacaccgcaggagggttttagagctagaaatagcaagttaaaataaggctagtccgttatcaacttgaaaaagtggcaccgagtcggtgc

SEQ ID NO: 17 - DNA, 176 bp, Artificial Sequence
  Description of Artificial Sequence Synthetic polynucleotide
aacaaagcaccagtggtctagtggtagaatagtaccctgccacggtacagacccgggttcgattcccggctggtgcaccaggatatttgcgtccctgggggttttagagctagaaatagcaagttaaaataaggctagtccgttatcaacttgaaaaagtggcaccgagtcggtgc

SEQ ID NO: 18 - DNA, 176 bp, Artificial Sequence
  Description of Artificial Sequence Synthetic polynucleotide
aacaaagcaccagtggtctagtggtagaatagtaccctgccacggtacagacccgggttcgattcccggctggtgcattctggatccctcaccccggcgggttttagagctagaaatagcaagttaaaataaggctagtccgttatcaacttgaaaaagtggcaccgagtcggtgc

SEQ ID NO: 19 - DNA, 328 bp, Artificial Sequence
  Description of Artificial Sequence Synthetic polynucleotide
aagaacgaactaagccggacaagaaaaggagcacatatacaaaccggttttattcatgaatggtcacgatggatgatggggctcagacttgagctacgaggccgcaggcgagagaagcctagtgtgctctctgcttgtttgggccgtaacggaggatacggccgacgagcgtgtactaccgcgcgggatgccgctgggcgctgcgggggccgttggatggggatcggtgggtcgcgggagcgttgaggggagacaggtttagtaccacctcgcctaccgaacaatgaagaacccaccttataaccccgcgcgctgccgcttgtgttag

SEQ ID NO: 20 - DNA, 20 bp, Artificial Sequence
  Description of Artificial Sequence Synthetic oligonucleotide
cactctccgactctacgcct

SEQ ID NO: 21 - DNA, 20 bp, Artificial Sequence
  Description of Artificial Sequence Synthetic oligonucleotide
taaacttctcggggcgaagc

SEQ ID NO: 22 - DNA, 20 bp, Artificial Sequence
  Description of Artificial Sequence Synthetic oligonucleotide
ctcgccagtgatggaggatg

SEQ ID NO: 23 - DNA, 20 bp, Artificial Sequence
  Description of Artificial Sequence Synthetic oligonucleotide
ccgtaggttgctagtccgtc

SEQ ID NO: 24 - DNA, 23 bp, Artificial Sequence
  Description of Artificial Sequence Synthetic oligonucleotide
cgactctacgcctttgatgaagg

SEQ ID NO: 25 - DNA, 23 bp, Artificial Sequence
  Description of Artificial Sequence Synthetic oligonucleotide
gcctttgatgaaggatgtgcagg

SEQ ID NO: 26 - DNA, 23 bp, Artificial Sequence
  Description of Artificial Sequence Synthetic oligonucleotide
cctttgatgaaggatgtgcaggg

SEQ ID NO: 27 - DNA, 23 bp, Artificial Sequence
  Description of Artificial Sequence Synthetic oligonucleotide
ggatgtgcagggcatccgaaagg

SEQ ID NO: 28 - DNA, 23 bp, Artificial Sequence
  Description of Artificial Sequence Synthetic oligonucleotide
gggcatccgaaaggcccaaaagg

SEQ ID NO: 29 - DNA, 23 bp, Artificial Sequence
  Description of Artificial Sequence Synthetic oligonucleotide
cgaaaggcccaaaaggccgacgg

SEQ ID NO: 30 - DNA, 23 bp, Artificial Sequence
  Description of Artificial Sequence Synthetic oligonucleotide
cgacggaactgcgaccgtgatgg

SEQ ID NO: 31 - DNA, 23 bp, Artificial Sequence
  Description of Artificial Sequence Synthetic oligonucleotide
actgcgaccgtgatggccattgg

SEQ ID NO: 32 - DNA, 23 bp, Artificial Sequence
  Description of Artificial Sequence Synthetic oligonucleotide
ccctcctcatatcatttcgcagg

SEQ ID NO: 33 - DNA, 23 bp, Artificial Sequence
  Description of Artificial Sequence Synthetic oligonucleotide
catttcgcaggacagctacgcgg

SEQ ID NO: 34 - DNA, 23 bp, Artificial Sequence
  Description of Artificial Sequence Synthetic oligonucleotide
gctacgcggatttctacttccgg

SEQ ID NO: 35 - DNA, 23 bp, Artificial Sequence
  Description of Artificial Sequence Synthetic oligonucleotide
ctacgcggatttctacttccggg

SEQ ID NO: 36 - DNA, 23 bp, Artificial Sequence
  Description of Artificial Sequence Synthetic oligonucleotide
ggtcaccaacagcgagcacaagg

SEQ ID NO: 37 - DNA, 23 bp, Artificial Sequence
  Description of Artificial Sequence Synthetic oligonucleotide
atctgcaagaaaacgatgatagg

SEQ ID NO: 38 - DNA, 23 bp, Artificial Sequence
  Description of Artificial Sequence Synthetic oligonucleotide
gcgttactttaacttcgacgagg

SEQ ID NO: 39 - DNA, 23 bp, Artificial Sequence
  Description of Artificial Sequence Synthetic oligonucleotide
acaagcccagcctcaacgacagg

SEQ ID NO: 40 - DNA, 23 bp, Artificial Sequence
  Description of Artificial Sequence Synthetic oligonucleotide
aggcatgacatttgcattcctgg

SEQ ID NO: 41 - DNA, 23 bp, Artificial Sequence
  Description of Artificial Sequence Synthetic oligonucleotide
ggcatgacatttgcattcctggg

SEQ ID NO: 42 - DNA, 23 bp, Artificial Sequence
  Description of Artificial Sequence Synthetic oligonucleotide
gcatgacatttgcattcctgggg

SEQ ID NO: 43 - DNA, 23 bp, Artificial Sequence
  Description of Artificial Sequence Synthetic oligonucleotide
catttgcattcctggggtgccgg

SEQ ID NO: 44 - DNA, 23 bp, Artificial Sequence
  Description of Artificial Sequence Synthetic oligonucleotide
cattcctggggtgccggcgctgg

SEQ ID NO: 45 - DNA, 23 bp, Artificial Sequence
  Description of Artificial Sequence Synthetic oligonucleotide
attcctggggtgccggcgctggg

SEQ ID NO: 46 - DNA, 23 bp, Artificial Sequence
  Description of Artificial Sequence Synthetic oligonucleotide
ttcctggggtgccggcgctgggg

SEQ ID NO: 47 - DNA, 23 bp, Artificial Sequence
  Description of Artificial Sequence Synthetic oligonucleotide
tcctggggtgccggcgctggggg

SEQ ID NO: 48 - DNA, 23 bp, Artificial Sequence
  Description of Artificial Sequence Synthetic oligonucleotide
tggggtgccggcgctgggggcgg

SEQ ID NO: 49 - DNA, 23 bp, Artificial Sequence
  Description of Artificial Sequence Synthetic oligonucleotide
gccggcgctgggggcggaagcgg

SEQ ID NO: 50 - DNA, 23 bp, Artificial Sequence
  Description of Artificial Sequence Synthetic oligonucleotide
agcggctgttaaagccattgagg

SEQ ID NO: 51 - DNA, 23 bp, Artificial Sequence
  Description of Artificial Sequence Synthetic oligonucleotide
ctgttaaagccattgaggagtgg

SEQ ID NO: 52 - DNA, 23 bp, Artificial Sequence
  Description of Artificial Sequence Synthetic oligonucleotide
tgttaaagccattgaggagtggg

SEQ ID NO: 53 - DNA, 23 bp, Artificial Sequence
  Description of Artificial Sequence Synthetic oligonucleotide
gttaaagccattgaggagtgggg

SEQ ID NO: 54 - DNA, 23 bp, Artificial Sequence
  Description of Artificial Sequence Synthetic oligonucleotide
cacctcgtcttctgcaccagcgg

SEQ ID NO: 55 - DNA, 23 bp, Artificial Sequence
  Description of Artificial Sequence Synthetic oligonucleotide
ctcgtcttctgcaccagcggcgg

SEQ ID NO: 56 - DNA, 23 bp, Artificial Sequence
  Description of Artificial Sequence Synthetic oligonucleotide
ttccagtgcgctaagctccttgg

SEQ ID NO: 57 - DNA, 23 bp, Artificial Sequence
  Description of Artificial Sequence Synthetic oligonucleotide
caaatactgcatctacatgcagg

SEQ ID NO: 58 - DNA, 23 bp, Artificial Sequence
  Description of Artificial Sequence Synthetic oligonucleotide
aaatactgcatctacatgcaggg

SEQ ID NO: 59 - DNA, 23 bp, Artificial Sequence
  Description of Artificial Sequence Synthetic oligonucleotide
tacatgcagggatgctatgctgg

SEQ ID NO: 60 - DNA, 23 bp, Artificial Sequence
  Description of Artificial Sequence Synthetic oligonucleotide
atgcagggatgctatgctggtgg

SEQ ID NO: 61 - DNA, 23 bp, Artificial Sequence
  Description of Artificial Sequence Synthetic oligonucleotide
atgctggtggcaccgtcatgcgg

SEQ ID NO: 62 - DNA, 23 bp, Artificial Sequence
  Description of Artificial Sequence Synthetic oligonucleotide
caccgtcatgcggtatgccaagg

SEQ ID NO: 63 - DNA, 23 bp, Artificial Sequence
  Description of Artificial Sequence Synthetic oligonucleotide
catgcggtatgccaaggatctgg

SEQ ID NO: 64 - DNA, 23 bp, Artificial Sequence
  Description of Artificial Sequence Synthetic oligonucleotide
gatctggccgagaacaaccgtgg

SEQ ID NO: 65 - DNA, 23 bp, Artificial Sequence
  Description of Artificial Sequence Synthetic oligonucleotide
ccgtggtgctcgtgttctgatgg

SEQ ID NO: 66 - DNA, 23 bp, Artificial Sequence
  Description of Artificial Sequence Synthetic oligonucleotide
ctcaccatcattgcgctccgtgg

SEQ ID NO: 67 - DNA, 23 bp, Artificial Sequence
  Description of Artificial Sequence Synthetic oligonucleotide
tcccatatcgacaatgctatcgg

SEQ ID NO: 68 - DNA, 23 bp, Artificial Sequence
  Description of Artificial Sequence Synthetic oligonucleotide
gctatcggcaactctcttttcgg

SEQ ID NO: 69 - DNA, 23 bp, Artificial Sequence
  Description of Artificial Sequence Synthetic oligonucleotide
ggcaactctcttttcggagatgg

SEQ ID NO: 70 - DNA, 23 bp, Artificial Sequence
  Description of Artificial Sequence Synthetic oligonucleotide
ggagctgctgcgctgattgttgg

SEQ ID NO: 71 - DNA, 23 bp, Artificial Sequence
  Description of Artificial Sequence Synthetic oligonucleotide
gagctgctgcgctgattgttggg

SEQ ID NO: 72 - DNA, 23 bp, Artificial Sequence
  Description of Artificial Sequence Synthetic oligonucleotide
gttgggtcagaccccatcatcgg

SEQ ID NO: 73 - DNA, 23 bp, Artificial Sequence
  Description of Artificial Sequence Synthetic oligonucleotide
atgttatccatctccacctgagg

SEQ ID NO: 74 - DNA, 23 bp, Artificial Sequence
  Description of Artificial Sequence Synthetic oligonucleotide
tgttatccatctccacctgaggg

SEQ ID NO: 75 - DNA, 23 bp, Artificial Sequence
  Description of Artificial Sequence Synthetic oligonucleotide
ccatctccacctgagggagtcgg

SEQ ID NO: 76 - DNA, 23 bp, Artificial Sequence
  Description of Artificial Sequence Synthetic oligonucleotide
catctccacctgagggagtcggg

SEQ ID NO: 77 - DNA, 23 bp, Artificial Sequence
  Description of Artificial Sequence Synthetic oligonucleotide
tctgatgttctacatgaccaagg

SEQ ID NO: 78 - DNA, 23 bp, Artificial Sequence
  Description of Artificial Sequence Synthetic oligonucleotide
gaccatctccaataatatagagg

SEQ ID NO: 79 - DNA, 23 bp, Artificial Sequence
  Description of Artificial Sequence Synthetic oligonucleotide
ccttgttgatgtgtttaagtcgg

SEQ ID NO: 80 - DNA, 23 bp, Artificial Sequence
  Description of Artificial Sequence Synthetic oligonucleotide
tgttgatgtgtttaagtcggtgg

SEQ ID NO: 81 - DNA, 23 bp, Artificial Sequence
  Description of Artificial Sequence Synthetic oligonucleotide
gttgatgtgtttaagtcggtggg

SEQ ID NO: 82 - DNA, 23 bp, Artificial Sequence
  Description of Artificial Sequence Synthetic oligonucleotide
ttgatgtgtttaagtcggtgggg

SEQ ID NO: 83 - DNA, 23 bp, Artificial Sequence
  Description of Artificial Sequence Synthetic oligonucleotide
gtcggtggggatgactcctccgg

SEQ ID NO: 84 - DNA, 23 bp, Artificial Sequence
  Description of Artificial Sequence Synthetic oligonucleotide
ggtggggatgactcctccggagg

SEQ ID NO: 85 - DNA, 23 bp, Artificial Sequence
  Description of Artificial Sequence Synthetic oligonucleotide
ggatgactcctccggaggattgg

SEQ ID NO: 86 - DNA, 23 bp, Artificial Sequence
  Description of Artificial Sequence Synthetic oligonucleotide
aggattggaactctctcttctgg

SEQ ID NO: 87 - DNA, 23 bp, Artificial Sequence
  Description of Artificial Sequence Synthetic oligonucleotide
ctcttctggatccctcatcccgg

SEQ ID NO: 88 - DNA, 23 bp, Artificial Sequence
  Description of Artificial Sequence Synthetic oligonucleotide
ttctggatccctcatcccggtgg

SEQ ID NO: 89 - DNA, 23 bp, Artificial Sequence
  Description of Artificial Sequence Synthetic oligonucleotide
ggatccctcatcccggtggccgg

SEQ ID NO: 90 - DNA, 23 bp, Artificial Sequence
  Description of Artificial Sequence Synthetic oligonucleotide
gatccctcatcccggtggccggg

SEQ ID NO: 91 - DNA, 23 bp, Artificial Sequence
  Description of Artificial Sequence Synthetic oligonucleotide
ggctatcctcgaccaagttgagg

SEQ ID NO: 92 - DNA, 23 bp, Artificial Sequence
  Description of Artificial Sequence Synthetic oligonucleotide
gtgcgactcgaacagttctctgg

SEQ ID NO: 93 - DNA, 23 bp, Artificial Sequence
  Description of Artificial Sequence Synthetic oligonucleotide
tgcgactcgaacagttctctggg

SEQ ID NO: 94 - DNA, 23 bp, Artificial Sequence
  Description of Artificial Sequence Synthetic oligonucleotide
cgaacagttctctgggattatgg

SEQ ID NO: 95 - DNA, 23 bp, Artificial Sequence
  Description of Artificial Sequence Synthetic oligonucleotide
tgcatgtgtgctctttatactgg

SEQ ID NO: 96 - DNA, 23 bp, Artificial Sequence
  Description of Artificial Sequence Synthetic oligonucleotide
agaagaaaatctgctgctgaagg

SEQ ID NO: 97 - DNA, 23 bp, Artificial Sequence
  Description of Artificial Sequence Synthetic oligonucleotide
gctgaaggattagaaacctacgg

SEQ ID NO: 98 - DNA, 23 bp, Artificial Sequence
  Description of Artificial Sequence Synthetic oligonucleotide
aggattagaaacctacggagagg

SEQ ID NO: 99 - DNA, 23 bp, Artificial Sequence
  Description of Artificial Sequence Synthetic oligonucleotide
ggattagaaacctacggagaggg

SEQ ID NO: 100 - DNA, 23 bp, Artificial Sequence
  Description of Artificial Sequence Synthetic oligonucleotide
gattagaaacctacggagagggg

SEQ ID NO: 101 - DNA, 23 bp, Artificial Sequence
  Description of Artificial Sequence Synthetic oligonucleotide
agaaacctacggagaggggttgg

SEQ ID NO: 102 - DNA, 23 bp, Artificial Sequence
  Description of Artificial Sequence Synthetic oligonucleotide
cctacggagaggggttggagtgg

SEQ ID NO: 103 - DNA, 23 bp, Artificial Sequence
  Description of Artificial Sequence Synthetic oligonucleotide
ctacggagaggggttggagtggg

SEQ ID NO: 104 - DNA, 23 bp, Artificial Sequence
  Description of Artificial Sequence Synthetic oligonucleotide
tacggagaggggttggagtgggg

SEQ ID NO: 105 - DNA, 23 bp, Artificial Sequence
  Description of Artificial Sequence Synthetic oligonucleotide
ttggagtggggtgtcttgcttgg

SEQ ID NO: 106 - DNA, 23 bp, Artificial Sequence
  Description of Artificial Sequence Synthetic oligonucleotide
tggggtgtcttgcttggttttgg

SEQ ID NO: 107 - DNA, 23 bp, Artificial Sequence
  Description of Artificial Sequence Synthetic oligonucleotide
gtcttgcttggttttggaccagg

SEQ ID NO: 108 - DNA, 23 bp, Artificial Sequence
  Description of Artificial Sequence Synthetic oligonucleotide
caggaggcaggctatgaagaagg

SEQ ID NO: 109 - DNA, 23 bp, Artificial Sequence
  Description of Artificial Sequence Synthetic oligonucleotide
atagtttcaatagtcattcctgg

SEQ ID NO: 110 - DNA, 23 bp, Artificial Sequence
  Description of Artificial Sequence Synthetic oligonucleotide
ccactccaacccctctccgtagg

SEQ ID NO: 111 - DNA, 23 bp, Artificial Sequence
  Description of Artificial Sequence Synthetic oligonucleotide
tcgagtcgcactaaacttctcgg

SEQ ID NO: 112 - DNA, 23 bp, Artificial Sequence
  Description of Artificial Sequence Synthetic oligonucleotide
cgagtcgcactaaacttctcggg

SEQ ID NO: 113 - DNA, 23 bp, Artificial Sequence
  Description of Artificial Sequence Synthetic oligonucleotide
gagtcgcactaaacttctcgggg

SEQ ID NO: 114 - DNA, 23 bp, Artificial Sequence
  Description of Artificial Sequence Synthetic oligonucleotide
ctcggggcgaagcttcagcttgg

SEQ ID NO: 115 - DNA, 23 bp, Artificial Sequence
  Description of Artificial Sequence Synthetic oligonucleotide
gcttcagcttggcctcaacttgg

SEQ ID NO: 116 - DNA, 23 bp, Artificial Sequence
  Description of Artificial Sequence Synthetic oligonucleotide
gcttggcctcaacttggtcgagg

SEQ ID NO: 117 - DNA, 23 bp, Artificial Sequence
  Description of Artificial Sequence Synthetic oligonucleotide
caacttggtcgaggatagcccgg

SEQ ID NO: 118 - DNA, 23 bp, Artificial Sequence
  Description of Artificial Sequence Synthetic oligonucleotide
gtcgaggatagcccggccaccgg

SEQ ID NO: 119 - DNA, 23 bp, Artificial Sequence
  Description of Artificial Sequence Synthetic oligonucleotide
tcgaggatagcccggccaccggg

SEQ ID NO: 120 - DNA, 23 bp, Artificial Sequence
  Description of Artificial Sequence Synthetic oligonucleotide
atagcccggccaccgggatgagg

SEQ ID NO: 121 - DNA, 23 bp, Artificial Sequence
  Description of Artificial Sequence Synthetic oligonucleotide
tagcccggccaccgggatgaggg

SEQ ID NO: 122 - DNA, 23 bp, Artificial Sequence
  Description of Artificial Sequence Synthetic oligonucleotide
aagagagagttccaatcctccgg

SEQ ID NO: 123 - DNA, 23 bp, Artificial Sequence
  Description of Artificial Sequence Synthetic oligonucleotide
agagagttccaatcctccggagg

SEQ ID NO: 124 - DNA, 23 bp, Artificial Sequence
  Description of Artificial Sequence Synthetic oligonucleotide
ccgacttaaacacatcaacaagg

SEQ ID NO: 125 - DNA, 23 bp, Artificial Sequence
  Description of Artificial Sequence Synthetic oligonucleotide
cttaaacacatcaacaaggcagg

SEQ ID NO: 126 - DNA, 23 bp, Artificial Sequence
  Description of Artificial Sequence Synthetic oligonucleotide
aaggcaggcctctatattattgg

SEQ ID NO: 127 - DNA, 23 bp, Artificial Sequence
  Description of Artificial Sequence Synthetic oligonucleotide
ggcctctatattattggagatgg

SEQ ID NO: 128 - DNA, 23 bp, Artificial Sequence
  Description of Artificial Sequence Synthetic oligonucleotide
tatattattggagatggtcgcgg

SEQ ID NO: 129 - DNA, 23 bp, Artificial Sequence
  Description of Artificial Sequence Synthetic oligonucleotide
gatggtcgcggcgctgtccttgg

SEQ ID NO: 130 - DNA, 23 bp, Artificial Sequence
  Description of Artificial Sequence Synthetic oligonucleotide
acatcagacccgactccctcagg

SEQ ID NO: 131 - DNA, 23 bp, Artificial Sequence
  Description of Artificial Sequence Synthetic oligonucleotide
tcagacccgactccctcaggtgg

SEQ ID NO: 132 - DNA, 23 bp, Artificial Sequence
  Description of Artificial Sequence Synthetic oligonucleotide
ccgactccctcaggtggagatgg

SEQ ID NO: 133 - DNA, 23 bp, Artificial Sequence
  Description of Artificial Sequence Synthetic oligonucleotide
tggataacatcttcactgtttgg

SEQ ID NO: 134 - DNA, 23 bp, Artificial Sequence
  Description of Artificial Sequence Synthetic oligonucleotide
ggataacatcttcactgtttggg

SEQ ID NO: 135 - DNA, 23 bp, Artificial Sequence
  Description of Artificial Sequence Synthetic oligonucleotide
tgcgcagacaatctcgaacatgg

SEQ ID NO: 136 - DNA, 23 bp, Artificial Sequence
  Description of Artificial Sequence Synthetic oligonucleotide
gcgcagacaatctcgaacatggg

SEQ ID NO: 137 - DNA, 23 bp, Artificial Sequence
  Description of Artificial Sequence Synthetic oligonucleotide
gggcttctcgacaccgatgatgg

SEQ ID NO: 138 - DNA, 23 bp, Artificial Sequence
  Description of Artificial Sequence Synthetic oligonucleotide
ggcttctcgacaccgatgatggg

SEQ ID NO: 139 - DNA, 23 bp, Artificial Sequence
  Description of Artificial Sequence Synthetic oligonucleotide
gcttctcgacaccgatgatgggg

SEQ ID NO: 140 - DNA, 23 bp, Artificial Sequence
  Description of Artificial Sequence Synthetic oligonucleotide
tgccgatagcattgtcgatatgg

SEQ ID NO: 141 - DNA, 23 bp, Artificial Sequence
  Description of Artificial Sequence Synthetic oligonucleotide
gccgatagcattgtcgatatggg

SEQ ID NO: 142 - DNA, 23 bp, Artificial Sequence
  Description of Artificial Sequence Synthetic oligonucleotide
attgtcgatatgggaatcattgg

SEQ ID NO: 143 - DNA, 23 bp, Artificial Sequence
  Description of Artificial Sequence Synthetic oligonucleotide
ttgtcgatatgggaatcattggg

SEQ ID NO: 144 - DNA, 23 bp, Artificial Sequence
  Description of Artificial Sequence Synthetic oligonucleotide
tgtcgatatgggaatcattgggg

SEQ ID NO: 145 - DNA, 23 bp, Artificial Sequence
  Description of Artificial Sequence Synthetic oligonucleotide
tatgggaatcattggggccacgg

SEQ ID NO: 146 - DNA, 23 bp, Artificial Sequence
  Description of Artificial Sequence Synthetic oligonucleotide
ggggccacggagcgcaatgatgg

SEQ ID NO: 147 - DNA, 23 bp, Artificial Sequence
  Description of Artificial Sequence Synthetic oligonucleotide
ccatcagaacacgagcaccacgg

SEQ ID NO: 148 - DNA, 23 bp, Artificial Sequence
  Description of Artificial Sequence Synthetic oligonucleotide
acgagcaccacggttgttctcgg

SEQ ID NO: 149 - DNA, 23 bp, Artificial Sequence
  Description of Artificial Sequence Synthetic oligonucleotide
gttgttctcggccagatccttgg

SEQ ID NO: 150 - DNA, 23 bp, Artificial Sequence
  Description of Artificial Sequence Synthetic oligonucleotide
atccttggcataccgcatgacgg

SEQ ID NO: 151 - DNA, 23 bp, Artificial Sequence
  Description of Artificial Sequence Synthetic oligonucleotide
gatgcagtatttgttgacattgg

SEQ ID NO: 152 - DNA, 23 bp, Artificial Sequence
  Description of Artificial Sequence Synthetic oligonucleotide
agtatttgttgacattggtgcgg

SEQ ID NO: 153 - DNA, 23 bp, Artificial Sequence
  Description of Artificial Sequence Synthetic oligonucleotide
atttgttgacattggtgcggagg

SEQ ID NO: 154 - DNA, 23 bp, Artificial Sequence
  Description of Artificial Sequence Synthetic oligonucleotide
tgacattggtgcggaggccaagg

SEQ ID NO: 155 - DNA, 23 bp, Artificial Sequence
  Description of Artificial Sequence Synthetic oligonucleotide
ggccaaggagcttagcgcactgg

SEQ ID NO: 156 - DNA, 23 bp, Artificial Sequence
  Description of Artificial Sequence Synthetic oligonucleotide
gagcttagcgcactggaaatcgg

SEQ ID NO: 157 - DNA, 23 bp, Artificial Sequence
  Description of Artificial Sequence Synthetic oligonucleotide
gcgcactggaaatcggcactagg

SEQ ID NO: 158 - DNA, 23 bp, Artificial Sequence
  Description of Artificial Sequence Synthetic oligonucleotide
aggcatgtcaacaccgccgctgg

SEQ ID NO: 159 - DNA, 23 bp, Artificial Sequence
  Description of Artificial Sequence Synthetic oligonucleotide
cgccgctggtgcagaagacgagg

SEQ ID NO: 160 - DNA, 23 bp, Artificial Sequence
  Description of Artificial Sequence Synthetic oligonucleotide
aggtgagtgatctcagacttagg

SEQ ID NO: 161 - DNA, 23 bp, Artificial Sequence
  Description of Artificial Sequence Synthetic oligonucleotide
ggtgagtgatctcagacttaggg

SEQ ID NO: 162 - DNA, 23 bp, Artificial Sequence
  Description of Artificial Sequence Synthetic oligonucleotide
agggcgtccccactcctcaatgg

SEQ ID NO: 163 - DNA, 23 bp, Artificial Sequence
  Description of Artificial Sequence Synthetic oligonucleotide
gccgcttccgcccccagcgccgg

SEQ ID NO: 164 - DNA, 23 bp, Artificial Sequence
  Description of Artificial Sequence Synthetic oligonucleotide
gcccccagcgccggcaccccagg

SEQ ID NO: 165 - DNA, 23 bp, Artificial Sequence
  Description of Artificial Sequence Synthetic oligonucleotide
aaatgtcatgcctgtcgttgagg

SEQ ID NO: 166 - DNA, 23 bp, Artificial Sequence
  Description of Artificial Sequence Synthetic oligonucleotide
gtcatgcctgtcgttgaggctgg

SEQ ID NO: 167 - DNA, 23 bp, Artificial Sequence
  Description of Artificial Sequence Synthetic oligonucleotide
tcatgcctgtcgttgaggctggg

SEQ ID NO: 168 - DNA, 23 bp, Artificial Sequence
  Description of Artificial Sequence Synthetic oligonucleotide
cttgtcaaatgaagtaatgttgg

SEQ ID NO: 169 - DNA, 23 bp, Artificial Sequence
  Description of Artificial Sequence Synthetic oligonucleotide
ttgtcaaatgaagtaatgttggg

SEQ ID NO: 170 - DNA, 23 bp, Artificial Sequence
  Description of Artificial Sequence Synthetic oligonucleotide
ctcgaccttgtgctcgctgttgg

SEQ ID NO: 171 - DNA, 23 bp, Artificial Sequence
  Description of Artificial Sequence Synthetic oligonucleotide
tgtgctcgctgttggtgacccgg

SEQ ID NO: 172 - DNA, 23 bp, Artificial Sequence
  Description of Artificial Sequence Synthetic oligonucleotide
ctgtcctgcgaaatgatatgagg

SEQ ID NO: 173 - DNA, 23 bp, Artificial Sequence
  Description of Artificial Sequence Synthetic oligonucleotide
tcctgcgaaatgatatgaggagg

SEQ ID NO: 174 - DNA, 23 bp, Artificial Sequence
  Description of Artificial Sequence Synthetic oligonucleotide
cctgcgaaatgatatgaggaggg

SEQ ID NO: 175 - DNA, 23 bp, Artificial Sequence
  Description of Artificial Sequence Synthetic oligonucleotide
aggagggtgagctgttccaatgg

SEQ ID NO: 176 - DNA, 23 bp, Artificial Sequence
  Description of Artificial Sequence Synthetic oligonucleotide
agctgttccaatggccatcacgg

SEQ ID NO: 177 - DNA, 23 bp, Artificial Sequence
  Description of Artificial Sequence Synthetic oligonucleotide
catcacggtcgcagttccgtcgg

SEQ ID NO: 178 - DNA, 23 bp, Artificial Sequence
  Description of Artificial Sequence Synthetic oligonucleotide
tcgcagttccgtcggccttttgg

SEQ ID NO: 179 - DNA, 23 bp, Artificial Sequence
  Description of Artificial Sequence Synthetic oligonucleotide
cgcagttccgtcggccttttggg

SEQ ID NO: 180 - DNA, 23 bp, Artificial Sequence
  Description of Artificial Sequence Synthetic oligonucleotide
cgtcggccttttgggcctttcgg

SEQ ID NO: 181 - DNA, 23 bp, Artificial Sequence
  Description of Artificial Sequence Synthetic oligonucleotide
ccctgcacatccttcatcaaagg

SEQ ID NO: 182 - DNA, 23 bp, Artificial Sequence
  Description of Artificial Sequence Synthetic oligonucleotide
cttcatcaaaggcgtagagtcgg

SEQ ID NO: 183 - DNA, 24 bp, Artificial Sequence
  Description of Artificial Sequence Synthetic oligonucleotide
tttgatgaaggatgtgcagggcat

SEQ ID NO: 184 - DNA, 24 bp, Artificial Sequence
  Description of Artificial Sequence Synthetic oligonucleotide
tttcgcaggacagctacgcggatt

SEQ ID NO: 185 - DNA, 24 bp, Artificial Sequence
  Description of Artificial Sequence Synthetic oligonucleotide
tttctacttccgggtcaccaacag

SEQ ID NO: 186 - DNA, 24 bp, Artificial Sequence
  Description of Artificial Sequence Synthetic oligonucleotide
tttaacttcgacgaggagttcttg

SEQ ID NO: 187 - DNA, 24 bp, Artificial Sequence
  Description of Artificial Sequence Synthetic oligonucleotide
tttgacaagcccagcctcaacgac

SEQ ID NO: 188 - DNA, 24 bp, Artificial Sequence
  Description of Artificial Sequence Synthetic oligonucleotide
tttgcattcctggggtgccggcgc

SEQ ID NO: 189 - DNA, 24 bp, Artificial Sequence
  Description of Artificial Sequence Synthetic oligonucleotide
tttccagtgcgctaagctccttgg

SEQ ID NO: 190 - DNA, 24 bp, Artificial Sequence
  Description of Artificial Sequence Synthetic oligonucleotide
ttttcggagatggagctgctgcgc

SEQ ID NO: 191 - DNA, 24 bp, Artificial Sequence
  Description of Artificial Sequence Synthetic oligonucleotide
tttcggagatggagctgctgcgct

SEQ ID NO: 192 - DNA, 24 bp, Artificial Sequence
  Description of Artificial Sequence Synthetic oligonucleotide
tttaagtcggtggggatgactcct

SEQ ID NO: 193 - DNA, 24 bp, Artificial Sequence
  Description of Artificial Sequence Synthetic oligonucleotide
tttagtgcgactcgaacagttctc

SEQ ID NO: 194 - DNA, 24 bp, Artificial Sequence
  Description of Artificial Sequence Synthetic oligonucleotide
tttatactggatgaaatgagaaga

SEQ ID NO: 195 - DNA, 24 bp, Artificial Sequence
  Description of Artificial Sequence Synthetic oligonucleotide
ttttggaccaggaatgactattga

SEQ ID NO: 196 - DNA, 24 bp, Artificial Sequence
  Description of Artificial Sequence Synthetic oligonucleotide
tttggaccaggaatgactattgaa

SEQ ID NO: 197 - DNA, 24 bp, Artificial Sequence
  Description of Artificial Sequence Synthetic oligonucleotide
tttcaatagtcattcctggtccaa

SEQ ID NO: 198 - DNA, 24 bp, Artificial Sequence
  Description of Artificial Sequence Synthetic oligonucleotide
tttctaatccttcagcagcagatt

SEQ ID NO: 199 - DNA, 24 bp, Artificial Sequence
  Description of Artificial Sequence Synthetic oligonucleotide
ttttcttctcatttcatccagtat

SEQ ID NO: 200 - DNA, 24 bp, Artificial Sequence
  Description of Artificial Sequence Synthetic oligonucleotide
tttcttctcatttcatccagtata

SEQ ID NO: 201 - DNA, 24 bp, Artificial Sequence
  Description of Artificial Sequence Synthetic oligonucleotide
tttcatccagtataaagagcacac

SEQ ID NO: 202 - DNA, 24 bp, Artificial Sequence
  Description of Artificial Sequence Synthetic oligonucleotide
tttgggatcacagtctgctttgcg

SEQ ID NO: 203 - DNA, 24 bp, Artificial Sequence
  Description of Artificial Sequence Synthetic oligonucleotide
tttgcgcagacaatctcgaacatg

SEQ ID NO: 204 - DNA, 24 bp, Artificial Sequence
  Description of Artificial Sequence Synthetic oligonucleotide
tttgttgacattggtgcggaggcc

SEQ ID NO: 205 - DNA, 24 bp, Artificial Sequence
  Description of Artificial Sequence Synthetic oligonucleotide
tttaacagccgcttccgcccccag

SEQ ID NO: 206 - DNA, 24 bp, Artificial Sequence
  Description of Artificial Sequence Synthetic oligonucleotide
ttttcttgcagatgcgatcgaact

SEQ ID NO: 207 - DNA, 24 bp, Artificial Sequence
  Description of Artificial Sequence Synthetic oligonucleotide
tttcttgcagatgcgatcgaactt

SEQ ID NO: 208 - DNA, 24 bp, Artificial Sequence
  Description of Artificial Sequence Synthetic oligonucleotide
ttttgggcctttcggatgccctgc

SEQ ID NO: 209 - DNA, 24 bp, Artificial Sequence
  Description of Artificial Sequence Synthetic oligonucleotide
tttgggcctttcggatgccctgca

SEQ ID NO: 210 - DNA, 24 bp, Artificial Sequence
  Description of Artificial Sequence Synthetic oligonucleotide
tttcggatgccctgcacatccttc

SEQ ID NO: 211 - DNA, 23 bp, Artificial Sequence
  Description of Artificial Sequence Synthetic oligonucleotide
ttcactctccgactctacgcctt

SEQ ID NO: 212 - DNA, 23 bp, Artificial Sequence
  Description of Artificial Sequence Synthetic oligonucleotide
ctctccgactctacgcctttgat

SEQ ID NO: 213 - DNA, 23 bp, Artificial Sequence
  Description of Artificial Sequence Synthetic oligonucleotide
ctccgactctacgcctttgatga

SEQ ID NO: 214 - DNA, 23 bp, Artificial Sequence
  Description of Artificial Sequence Synthetic oligonucleotide
ctctacgcctttgatgaaggatg

SEQ ID NO: 215 - DNA, 23 bp, Artificial Sequence
  Description of Artificial Sequence Synthetic oligonucleotide
ctacgcctttgatgaaggatgtg

SEQ ID NO: 216 - DNA, 23 bp, Artificial Sequence
  Description of Artificial Sequence Synthetic oligonucleotide
ctttgatgaaggatgtgcagggc

SEQ ID NO: 217 - DNA, 23 bp, Artificial Sequence
  Description of Artificial Sequence Synthetic oligonucleotide
tttgatgaaggatgtgcagggca

SEQ ID NO: 218 - DNA, 23 bp, Artificial Sequence
  Description of Artificial Sequence Synthetic oligonucleotide
ttgatgaaggatgtgcagggcat

SEQ ID NO: 219 - DNA, 23 bp, Artificial Sequence
  Description of Artificial Sequence Synthetic oligonucleotide
ctgcgaccgtgatggccattgga

SEQ ID NO: 220 - DNA, 23 bp, Artificial Sequence
  Description of Artificial Sequence Synthetic oligonucleotide
ttggaacagctcaccctcctcat

SEQ ID NO: 221 - DNA, 23 bp, Artificial Sequence
  Description of Artificial Sequence Synthetic oligonucleotide
ctcaccctcctcatatcatttcg

SEQ ID NO: 222 - DNA, 23 bp, Artificial Sequence
  Description of Artificial Sequence Synthetic oligonucleotide
ctcctcatatcatttcgcaggac

SEQ ID NO: 223 - DNA, 23 bp, Artificial Sequence
  Description of Artificial Sequence Synthetic oligonucleotide
ctcatatcatttcgcaggacagc

SEQ ID NO: 224 - DNA, 23 bp, Artificial Sequence
  Description of Artificial Sequence Synthetic oligonucleotide
tttcgcaggacagctacgcggat

SEQ ID NO: 225 - DNA, 23 bp, Artificial Sequence
  Description of Artificial Sequence Synthetic oligonucleotide
ttcgcaggacagctacgcggatt

SEQ ID NO: 226 - DNA, 23 bp, Artificial Sequence
  Description of Artificial Sequence Synthetic oligonucleotide
tttctacttccgggtcaccaaca

SEQ ID NO: 227 - DNA, 23 bp, Artificial Sequence
  Description of Artificial Sequence Synthetic oligonucleotide
ttctacttccgggtcaccaacag

SEQ ID NO: 228 - DNA, 23 bp, Artificial Sequence
  Description of Artificial Sequence Synthetic oligonucleotide
ctacttccgggtcaccaacagcg

SEQ ID NO: 229 - DNA, 23 bp, Artificial Sequence
  Description of Artificial Sequence Synthetic oligonucleotide
cttccgggtcaccaacagcgagc

SEQ ID NO: 230 - DNA, 23 bp, Artificial Sequence
  Description of Artificial Sequence Synthetic oligonucleotide
ttccgggtcaccaacagcgagca

SEQ ID NO: 231 - DNA, 23 bp, Artificial Sequence
  Description of Artificial Sequence Synthetic oligonucleotide
ctcaagaagaagttcgatcgcat

SEQ ID NO: 232 - DNA, 23 bp, Artificial Sequence
  Description of Artificial Sequence Synthetic oligonucleotide
ttcgatcgcatctgcaagaaaac

SEQ ID NO: 233 - DNA, 23 bp, Artificial Sequence
  Description of Artificial Sequence Synthetic oligonucleotide
ctgcaagaaaacgatgataggca

SEQ ID NO: 234 - DNA, 23 bp, Artificial Sequence
  Description of Artificial Sequence Synthetic oligonucleotide
ttactttaacttcgacgaggagt

SEQ ID NO: 235 - DNA, 23 bp, Artificial Sequence
  Description of Artificial Sequence Synthetic oligonucleotide
ctttaacttcgacgaggagttct

SEQ ID NO: 236 - DNA, 23 bp, Artificial Sequence
  Description of Artificial Sequence Synthetic oligonucleotide
tttaacttcgacgaggagttctt

SEQ ID NO: 237 - DNA, 23 bp, Artificial Sequence
  Description of Artificial Sequence Synthetic oligonucleotide
ttaacttcgacgaggagttcttg

SEQ ID NO: 238 - DNA, 23 bp, Artificial Sequence
  Description of Artificial Sequence Synthetic oligonucleotide
cttcgacgaggagttcttgaaga

SEQ ID NO: 239 - DNA, 23 bp, Artificial Sequence
  Description of Artificial Sequence Synthetic oligonucleotide
ttcgacgaggagttcttgaagaa

SEQ ID NO: 240 - DNA, 23 bp, Artificial Sequence
  Description of Artificial Sequence Synthetic oligonucleotide
ttcttgaagaagtatcccaacat

SEQ ID NO: 241 - DNA, 23 bp, Artificial Sequence
  Description of Artificial Sequence Synthetic oligonucleotide
cttgaagaagtatcccaacatta

SEQ ID NO: 242 - DNA, 23 bp, Artificial Sequence
  Description of Artificial Sequence Synthetic oligonucleotide
ttgaagaagtatcccaacattac

SEQ ID NO: 243 - DNA, 23 bp, Artificial Sequence
  Description of Artificial Sequence Synthetic oligonucleotide
ttacttcatttgacaagcccagc

SEQ ID NO: 244 - DNA, 23 bp, Artificial Sequence
  Description of Artificial Sequence Synthetic oligonucleotide
cttcatttgacaagcccagcctc

SEQ ID NO: 245 - DNA, 23 bp, Artificial Sequence
  Description of Artificial Sequence Synthetic oligonucleotide
ttcatttgacaagcccagcctca

SEQ ID NO: 246 - DNA, 23 bp, Artificial Sequence
  Description of Artificial Sequence Synthetic oligonucleotide
tttgacaagcccagcctcaacga

SEQ ID NO: 247 - DNA, 23 bp, Artificial Sequence
  Description of Artificial Sequence Synthetic oligonucleotide
ttgacaagcccagcctcaacgac

SEQ ID NO: 248 - DNA, 23 bp, Artificial Sequence
  Description of Artificial Sequence Synthetic oligonucleotide
ctcaacgacaggcatgacatttg

SEQ ID NO: 249 - DNA, 23 bp, Artificial Sequence
  Description of Artificial Sequence Synthetic oligonucleotide
tttgcattcctggggtgccggcg

SEQ ID NO: 250 - DNA, 23 bp, Artificial Sequence
  Description of Artificial Sequence Synthetic oligonucleotide
ttgcattcctggggtgccggcgc

SEQ ID NO: 251 - DNA, 23 bp, Artificial Sequence
  Description of Artificial Sequence Synthetic oligonucleotide
ctggggtgccggcgctgggggcg

SEQ ID NO: 252 - DNA, 23 bp, Artificial Sequence
  Description of Artificial Sequence Synthetic oligonucleotide
ctgggggcggaagcggctgttaa

SEQ ID NO: 253 - DNA, 23 bp, Artificial Sequence
  Description of Artificial Sequence Synthetic oligonucleotide
ttaaagccattgaggagtgggga

SEQ ID NO: 254 - DNA, 23 bp, Artificial Sequence
  Description of Artificial Sequence Synthetic oligonucleotide
ttgaggagtggggacgccctaag

SEQ ID NO: 255 - DNA, 23 bp, Artificial Sequence
  Description of Artificial Sequence Synthetic oligonucleotide
ctaagtctgagatcactcacctc

SEQ ID NO: 256 - DNA, 23 bp, Artificial Sequence
  Description of Artificial Sequence Synthetic oligonucleotide
ctgagatcactcacctcgtcttc

SEQ ID NO: 257 - DNA, 23 bp, Artificial Sequence
  Description of Artificial Sequence Synthetic oligonucleotide
ctcacctcgtcttctgcaccagc

SEQ ID NO: 258 - DNA, 23 bp, Artificial Sequence
  Description of Artificial Sequence Synthetic oligonucleotide
cttctgcaccagcggcggtgttg

SEQ ID NO: 259 - DNA, 23 bp, Artificial Sequence
  Description of Artificial Sequence Synthetic oligonucleotide
ttctgcaccagcggcggtgttga

SEQ ID NO: 260 - DNA, 23 bp, Artificial Sequence
  Description of Artificial Sequence Synthetic oligonucleotide
ctgcaccagcggcggtgttgaca

SEQ ID NO: 261 - DNA, 23 bp, Artificial Sequence
  Description of Artificial Sequence Synthetic oligonucleotide
ttgacatgcctagtgccgatttc

SEQ ID NO: 262 - DNA, 23 bp, Artificial Sequence
  Description of Artificial Sequence Synthetic oligonucleotide
ctagtgccgatttccagtgcgct

SEQ ID NO: 263 - DNA, 23 bp, Artificial Sequence
  Description of Artificial Sequence Synthetic oligonucleotide
tttccagtgcgctaagctccttg

SEQ ID NO: 264 - DNA, 23 bp, Artificial Sequence
  Description of Artificial Sequence Synthetic oligonucleotide
ctaagctccttggcctccgcacc

SEQ ID NO: 265 - DNA, 23 bp, Artificial Sequence
  Description of Artificial Sequence Synthetic oligonucleotide
ctccttggcctccgcaccaatgt

SEQ ID NO: 266 - DNA, 23 bp, Artificial Sequence
  Description of Artificial Sequence Synthetic oligonucleotide
cttggcctccgcaccaatgtcaa

SEQ ID NO: 267 - DNA, 23 bp, Artificial Sequence
  Description of Artificial Sequence Synthetic oligonucleotide
ttggcctccgcaccaatgtcaac

SEQ ID NO: 268 - DNA, 23 bp, Artificial Sequence
  Description of Artificial Sequence Synthetic oligonucleotide
ctccgcaccaatgtcaacaaata

SEQ ID NO: 269 - DNA, 23 bp, Artificial Sequence
  Description of Artificial Sequence Synthetic oligonucleotide
ctgcatctacatgcagggatgct

SEQ ID NO: 270 - DNA, 23 bp, Artificial Sequence
  Description of Artificial Sequence Synthetic oligonucleotide
ctacatgcagggatgctatgctg

SEQ ID NO: 271 - DNA, 23 bp, Artificial Sequence
  Description of Artificial Sequence Synthetic oligonucleotide
ctatgctggtggcaccgtcatgc

SEQ ID NO: 272 - DNA, 23 bp, Artificial Sequence
  Description of Artificial Sequence Synthetic oligonucleotide
ctggtggcaccgtcatgcggtat

SEQ ID NO: 273 - DNA, 23 bp, Artificial Sequence
  Description of Artificial Sequence Synthetic oligonucleotide
ctggccgagaacaaccgtggtgc

SEQ ID NO: 274 - DNA, 23 bp, Artificial Sequence
  Description of Artificial Sequence Synthetic oligonucleotide
ctcgtgttctgatggtgtgcgca

SEQ ID NO: 275 - DNA, 23 bp, Artificial Sequence
  Description of Artificial Sequence Synthetic oligonucleotide
ttctgatggtgtgcgcagagctc

SEQ ID NO: 276 - DNA, 23 bp, Artificial Sequence
  Description of Artificial Sequence Synthetic oligonucleotide
ctgatggtgtgcgcagagctcac

SEQ ID NO: 277 - DNA, 23 bp, Artificial Sequence
  Description of Artificial Sequence Synthetic oligonucleotide
ttgcgctccgtggccccaatgat

SEQ ID NO: 278 - DNA, 23 bp, Artificial Sequence
  Description of Artificial Sequence Synthetic oligonucleotide
ctccgtggccccaatgattccca

SEQ ID NO: 279 - DNA, 23 bp, Artificial Sequence
  Description of Artificial Sequence Synthetic oligonucleotide
ttcccatatcgacaatgctatcg

SEQ ID NO: 280 - DNA, 23 bp, Artificial Sequence
  Description of Artificial Sequence Synthetic oligonucleotide
ctatcggcaactctcttttcgga

SEQ ID NO: 281 - DNA, 23 bp, Artificial Sequence
  Description of Artificial Sequence Synthetic oligonucleotide
ctctcttttcggagatggagctg

SEQ ID NO: 282 - DNA, 23 bp, Artificial Sequence
  Description of Artificial Sequence Synthetic oligonucleotide
ctcttttcggagatggagctgct

SEQ ID NO: 283 - DNA, 23 bp, Artificial Sequence
  Description of Artificial Sequence Synthetic oligonucleotide
cttttcggagatggagctgctgc

SEQ ID NO: 284 - DNA, 23 bp, Artificial Sequence
  Description of Artificial Sequence Synthetic oligonucleotide
ttttcggagatggagctgctgcg

SEQ ID NO: 285 - DNA, 23 bp, Artificial Sequence
  Description of Artificial Sequence Synthetic oligonucleotide
tttcggagatggagctgctgcgc

SEQ ID NO: 286 - DNA, 23 bp, Artificial Sequence
  Description of Artificial Sequence Synthetic oligonucleotide
ttcggagatggagctgctgcgct

SEQ ID NO: 287 - DNA, 23 bp, Artificial Sequence
  Description of Artificial Sequence Synthetic oligonucleotide
ctgctgcgctgattgttgggtca

SEQ ID NO: 288 - DNA, 23 bp, Artificial Sequence
  Description of Artificial Sequence Synthetic oligonucleotide
ctgcgctgattgttgggtcagac

SEQ ID NO: 289 - DNA, 23 bp, Artificial Sequence
  Description of Artificial Sequence Synthetic oligonucleotide
ctgattgttgggtcagaccccat

SEQ ID NO: 290 - DNA, 23 bp, Artificial Sequence
  Description of Artificial Sequence Synthetic oligonucleotide
ttgttgggtcagaccccatcatc

SEQ ID NO: 291 - DNA, 23 bp, Artificial Sequence
  Description of Artificial Sequence Synthetic oligonucleotide
ttgggtcagaccccatcatcggt

SEQ ID NO: 292 - DNA, 23 bp, Artificial Sequence
  Description of Artificial Sequence Synthetic oligonucleotide
ttcgagattgtctgcgcaaagca

SEQ ID NO: 293 - DNA, 23 bp, Artificial Sequence
  Description of Artificial Sequence Synthetic oligonucleotide
ttgtctgcgcaaagcagactgtg

SEQ ID NO: 294 - DNA, 23 bp, Artificial Sequence
  Description of Artificial Sequence Synthetic oligonucleotide
ctgcgcaaagcagactgtgatcc

SEQ ID NO: 295 - DNA, 23 bp, Artificial Sequence
  Description of Artificial Sequence Synthetic oligonucleotide
ctgtgatcccaaacagtgaagat

SEQ ID NO: 296 - DNA, 23 bp, Artificial Sequence
  Description of Artificial Sequence Synthetic oligonucleotide
ttatccatctccacctgagggag

SEQ ID NO: 297 - DNA, 23 bp, Artificial Sequence
  Description of Artificial Sequence Synthetic oligonucleotide
ctccacctgagggagtcgggtct

SEQ ID NO: 298 - DNA, 23 bp, Artificial Sequence
  Description of Artificial Sequence Synthetic oligonucleotide
ctgagggagtcgggtctgatgtt

SEQ ID NO: 299 - DNA, 23 bp, Artificial Sequence
  Description of Artificial Sequence Synthetic oligonucleotide
ctgatgttctacatgaccaagga

SEQ ID NO: 300 - DNA, 23 bp, Artificial Sequence
  Description of Artificial Sequence Synthetic oligonucleotide
ttctacatgaccaaggacagcgc

SEQ ID NO: 301 - DNA, 23 bp, Artificial Sequence
  Description of Artificial Sequence Synthetic oligonucleotide
ctacatgaccaaggacagcgccg

SEQ ID NO: 302 - DNA, 23 bp, Artificial Sequence
  Description of Artificial Sequence Synthetic oligonucleotide
ctccaataatatagaggcctgcc

SEQ ID NO: 303 - DNA, 23 bp, Artificial Sequence
  Description of Artificial Sequence Synthetic oligonucleotide
ctgccttgttgatgtgtttaagt

SEQ ID NO: 304 - DNA, 23 bp, Artificial Sequence
  Description of Artificial Sequence Synthetic oligonucleotide
cttgttgatgtgtttaagtcggt

SEQ ID NO: 305 - DNA, 23 bp, Artificial Sequence
  Description of Artificial Sequence Synthetic oligonucleotide
ttgttgatgtgtttaagtcggtg

SEQ ID NO: 306 - DNA, 23 bp, Artificial Sequence
  Description of Artificial Sequence Synthetic oligonucleotide
tttaagtcggtggggatgactcc

SEQ ID NO: 307 - DNA, 23 bp, Artificial Sequence
  Description of Artificial Sequence Synthetic oligonucleotide
ttaagtcggtggggatgactcct

SEQ ID NO: 308 - DNA, 23 bp, Artificial Sequence
  Description of Artificial Sequence Synthetic oligonucleotide
ctcctccggaggattggaactct

SEQ ID NO: 309 - DNA, 23 bp, Artificial Sequence
  Description of Artificial Sequence Synthetic oligonucleotide
ctccggaggattggaactctctc

SEQ ID NO: 310 - DNA, 23 bp, Artificial Sequence
  Description of Artificial Sequence Synthetic oligonucleotide
ttggaactctctcttctggatcc

SEQ ID NO: 311 - DNA, 23 bp, Artificial Sequence
  Description of Artificial Sequence Synthetic oligonucleotide
ctctctcttctggatccctcatc

SEQ ID NO: 312 - DNA, 23 bp, Artificial Sequence
  Description of Artificial Sequence Synthetic oligonucleotide
ctctcttctggatccctcatccc

SEQ ID NO: 313 - DNA, 23 bp, Artificial Sequence
  Description of Artificial Sequence Synthetic oligonucleotide
cttctggatccctcatcccggtg

SEQ ID NO: 314 - DNA, 23 bp, Artificial Sequence
  Description of Artificial Sequence Synthetic oligonucleotide
ctggatccctcatcccggtggcc

SEQ ID NO: 315 - DNA, 23 bp, Artificial Sequence
  Description of Artificial Sequence Synthetic oligonucleotide
ctcatcccggtggccgggctatc

SEQ ID NO: 316 - DNA, 23 bp, Artificial Sequence
  Description of Artificial Sequence Synthetic oligonucleotide
ctatcctcgaccaagttgaggcc

SEQ ID NO: 317 - DNA, 23 bp, Artificial Sequence
  Description of Artificial Sequence Synthetic oligonucleotide
ctcgaccaagttgaggccaagct

SEQ ID NO: 318 - DNA, 23 bp, Artificial Sequence
  Description of Artificial Sequence Synthetic oligonucleotide
ttgaggccaagctgaagcttcgc

SEQ ID NO: 319 - DNA, 23 bp, Artificial Sequence
  Description of Artificial Sequence Synthetic oligonucleotide
ctgaagcttcgccccgagaagtt

SEQ ID NO: 320 - DNA, 23 bp, Artificial Sequence
  Description of Artificial Sequence Synthetic oligonucleotide
cttcgccccgagaagtttagtgc

SEQ ID NO: 321 - DNA, 23 bp, Artificial Sequence
  Description of Artificial Sequence Synthetic oligonucleotide
ttcgccccgagaagtttagtgcg

SEQ ID NO: 322 - DNA, 23 bp, Artificial Sequence
  Description of Artificial Sequence Synthetic oligonucleotide
tttagtgcgactcgaacagttct

SEQ ID NO: 323 - DNA, 23 bp, Artificial Sequence
  Description of Artificial Sequence Synthetic oligonucleotide
ttagtgcgactcgaacagttctc

SEQ ID NO: 324 - DNA, 23 bp, Artificial Sequence
  Description of Artificial Sequence Synthetic oligonucleotide
ctcgaacagttctctgggattat

SEQ ID NO: 325 - DNA, 23 bp, Artificial Sequence
  Description of Artificial Sequence Synthetic oligonucleotide
ttctctgggattatggcaacatg

SEQ ID NO: 326 - DNA, 23 bp, Artificial Sequence
  Description of Artificial Sequence Synthetic oligonucleotide
ctctgggattatggcaacatgat

SEQ ID NO: 327 - DNA, 23 bp, Artificial Sequence
  Description of Artificial Sequence Synthetic oligonucleotide
ctgggattatggcaacatgataa

SEQ ID NO: 328 - DNA, 23 bp, Artificial Sequence
  Description of Artificial Sequence Synthetic oligonucleotide
ttatggcaacatgataagtgcat

SEQ ID NO: 329 - DNA, 23 bp, Artificial Sequence
  Description of Artificial Sequence Synthetic oligonucleotide
ctctttatactggatgaaatgag

SEQ ID NO: 330 - DNA, 23 bp, Artificial Sequence
  Description of Artificial Sequence Synthetic oligonucleotide
ctttatactggatgaaatgagaa

SEQ ID NO: 331 - DNA, 23 bp, Artificial Sequence
  Description of Artificial Sequence Synthetic oligonucleotide
tttatactggatgaaatgagaag

SEQ ID NO: 332 - DNA, 23 bp, Artificial Sequence
  Description of Artificial Sequence Synthetic oligonucleotide
ttatactggatgaaatgagaaga

SEQ ID NO: 333 - DNA, 23 bp, Artificial Sequence
  Description of Artificial Sequence Synthetic oligonucleotide
ctggatgaaatgagaagaaaatc

SEQ ID NO: 334 - DNA, 23 bp, Artificial Sequence
  Description of Artificial Sequence Synthetic oligonucleotide
ctgctgctgaaggattagaaacc

SEQ ID NO: 335 - DNA, 23 bp, Artificial Sequence
  Description of Artificial Sequence Synthetic oligonucleotide
ctgctgaaggattagaaacctac

SEQ ID NO: 336 - DNA, 23 bp, Artificial Sequence
  Description of Artificial Sequence Synthetic oligonucleotide
ctgaaggattagaaacctacgga

SEQ ID NO: 337 - DNA, 23 bp, Artificial Sequence
  Description of Artificial Sequence Synthetic oligonucleotide
ttagaaacctacggagaggggtt

SEQ ID NO: 338 - DNA, 23 bp, Artificial Sequence
  Description of Artificial Sequence Synthetic oligonucleotide
cttgcttggttttggaccaggaa

SEQ ID NO: 339 - DNA, 23 bp, Artificial Sequence
  Description of Artificial Sequence Synthetic oligonucleotide
ttgcttggttttggaccaggaat

SEQ ID NO: 340 - DNA, 23 bp, Artificial Sequence
  Description of Artificial Sequence Synthetic oligonucleotide
cttggttttggaccaggaatgac

SEQ ID NO: 341 - DNA, 23 bp, Artificial Sequence
  Description of Artificial Sequence Synthetic oligonucleotide
ttggttttggaccaggaatgact

SEQ ID NO: 342 - DNA, 23 bp, Artificial Sequence
  Description of Artificial Sequence Synthetic oligonucleotide
ttttggaccaggaatgactattg

SEQ ID NO: 343 - DNA, 23 bp, Artificial Sequence
  Description of Artificial Sequence Synthetic oligonucleotide
tttggaccaggaatgactattga

SEQ ID NO: 344 - DNA, 23 bp, Artificial Sequence
  Description of Artificial Sequence Synthetic oligonucleotide
ttggaccaggaatgactattgaa

SEQ ID NO: 345 - DNA, 23 bp, Artificial Sequence
  Description of Artificial Sequence Synthetic oligonucleotide
ctattgaaactatccttcttcat

SEQ ID NO: 346 - DNA, 23 bp, Artificial Sequence
  Description of Artificial Sequence Synthetic oligonucleotide
ttgaaactatccttcttcatagc

SEQ ID NO: 347 - DNA, 23 bp, Artificial Sequence
  Description of Artificial Sequence Synthetic oligonucleotide
ctatccttcttcatagcctgcct

SEQ ID NO: 348 - DNA, 23 bp, Artificial Sequence
  Description of Artificial Sequence Synthetic oligonucleotide
cttcttcatagcctgcctcctgt

SEQ ID NO: 349 - DNA, 23 bp, Artificial Sequence
  Description of Artificial Sequence Synthetic oligonucleotide
ttcttcatagcctgcctcctgtg

SEQ ID NO: 350 - DNA, 23 bp, Artificial Sequence
  Description of Artificial Sequence Synthetic oligonucleotide
cttcatagcctgcctcctgtgta

SEQ ID NO: 351 - DNA, 23 bp, Artificial Sequence
  Description of Artificial Sequence Synthetic oligonucleotide
ttcatagcctgcctcctgtgtaa

SEQ ID NO: 352 - DNA, 23 bp, Artificial Sequence
  Description of Artificial Sequence Synthetic oligonucleotide
ttacacaggaggcaggctatgaa

SEQ ID NO: 353 - DNA, 23 bp, Artificial Sequence
  Description of Artificial Sequence Synthetic oligonucleotide
ctatgaagaaggatagtttcaat

SEQ ID NO: 354 - DNA, 23 bp, Artificial Sequence
  Description of Artificial Sequence Synthetic oligonucleotide
tttcaatagtcattcctggtcca

SEQ ID NO: 355 - DNA, 23 bp, Artificial Sequence
  Description of Artificial Sequence Synthetic oligonucleotide
ttcaatagtcattcctggtccaa

SEQ ID NO: 356 - DNA, 23 bp, Artificial Sequence
  Description of Artificial Sequence Synthetic oligonucleotide
ttcctggtccaaaaccaagcaag

SEQ ID NO: 357 - DNA, 23 bp, Artificial Sequence
  Description of Artificial Sequence Synthetic oligonucleotide
ctggtccaaaaccaagcaagaca

SEQ ID NO: 358 - DNA, 23 bp, Artificial Sequence
  Description of Artificial Sequence Synthetic oligonucleotide
ctccaacccctctccgtaggttt

SEQ ID NO: 359 - DNA, 23 bp, Artificial Sequence
  Description of Artificial Sequence Synthetic oligonucleotide
ctctccgtaggtttctaatcctt

SEQ ID NO: 360 - DNA, 23 bp, Artificial Sequence
  Description of Artificial Sequence Synthetic oligonucleotide
ctccgtaggtttctaatccttca

SEQ ID NO: 361 - DNA, 23 bp, Artificial Sequence
  Description of Artificial Sequence Synthetic oligonucleotide
tttctaatccttcagcagcagat

SEQ ID NO: 362 - DNA, 23 bp, Artificial Sequence
  Description of Artificial Sequence Synthetic oligonucleotide
ttctaatccttcagcagcagatt

SEQ ID NO: 363 - DNA, 23 bp, Artificial Sequence
  Description of Artificial Sequence Synthetic oligonucleotide
ctaatccttcagcagcagatttt

SEQ ID NO: 364 - DNA, 23 bp, Artificial Sequence
  Description of Artificial Sequence Synthetic oligonucleotide
cttcagcagcagattttcttctc

SEQ ID NO: 365 - DNA, 23 bp, Artificial Sequence
  Description of Artificial Sequence Synthetic oligonucleotide
ttcagcagcagattttcttctca

SEQ ID NO: 366 - DNA, 23 bp, Artificial Sequence
  Description of Artificial Sequence Synthetic oligonucleotide
ttttcttctcatttcatccagta

SEQ ID NO: 367 - DNA, 23 bp, Artificial Sequence
  Description of Artificial Sequence Synthetic oligonucleotide
tttcttctcatttcatccagtat

SEQ ID NO: 368 - DNA, 23 bp, Artificial Sequence
  Description of Artificial Sequence Synthetic oligonucleotide
ttcttctcatttcatccagtata

SEQ ID NO: 369 - DNA, 23 bp, Artificial Sequence
  Description of Artificial Sequence Synthetic oligonucleotide
cttctcatttcatccagtataaa

SEQ ID NO: 370 - DNA, 23 bp, Artificial Sequence
  Description of Artificial Sequence Synthetic oligonucleotide
ttctcatttcatccagtataaag

SEQ ID NO: 371 - DNA, 23 bp, Artificial Sequence
  Description of Artificial Sequence Synthetic oligonucleotide
ctcatttcatccagtataaagag

SEQ ID NO: 372 - DNA, 23 bp, Artificial Sequence
  Description of Artificial Sequence Synthetic oligonucleotide
tttcatccagtataaagagcaca

SEQ ID NO: 373 - DNA, 23 bp, Artificial Sequence
  Description of Artificial Sequence Synthetic oligonucleotide
ttcatccagtataaagagcacac

SEQ ID NO: 374 - DNA, 23 bp, Artificial Sequence
  Description of Artificial Sequence Synthetic oligonucleotide
cttatcatgttgccataatccca

SEQ ID NO: 375 - DNA, 23 bp, Artificial Sequence
  Description of Artificial Sequence Synthetic oligonucleotide
ttatcatgttgccataatcccag

SEQ ID NO: 376 - DNA, 23 bp, Artificial Sequence
  Description of Artificial Sequence Synthetic oligonucleotide
ttgccataatcccagagaactgt

SEQ ID NO: 377 - DNA, 23 bp, Artificial Sequence
  Description of Artificial Sequence Synthetic oligonucleotide
ctgttcgagtcgcactaaacttc

SEQ ID NO: 378 - DNA, 23 bp, Artificial Sequence
  Description of Artificial Sequence Synthetic oligonucleotide
ttcgagtcgcactaaacttctcg

SEQ ID NO: 379 - DNA, 23 bp, Artificial Sequence
  Description of Artificial Sequence Synthetic oligonucleotide
ctaaacttctcggggcgaagctt

SEQ ID NO: 380 - DNA, 23 bp, Artificial Sequence
  Description of Artificial Sequence Synthetic oligonucleotide
cttctcggggcgaagcttcagct

SEQ ID NO: 381 - DNA, 23 bp, Artificial Sequence
  Description of Artificial Sequence Synthetic oligonucleotide
ttctcggggcgaagcttcagctt

SEQ ID NO: 382 - DNA, 23 bp, Artificial Sequence
  Description of Artificial Sequence Synthetic oligonucleotide
cttcagcttggcctcaacttggt

SEQ ID NO: 383 - DNA, 23 bp, Artificial Sequence
  Description of Artificial Sequence Synthetic oligonucleotide
ttcagcttggcctcaacttggtc

SEQ ID NO: 384 - DNA, 23 bp, Artificial Sequence
  Description of Artificial Sequence Synthetic oligonucleotide
cttggcctcaacttggtcgagga

SEQ ID NO: 385 - DNA, 23 bp, Artificial Sequence
  Description of Artificial Sequence Synthetic oligonucleotide
ttggcctcaacttggtcgaggat

SEQ ID NO: 386 - DNA, 23 bp, Artificial Sequence
  Description of Artificial Sequence Synthetic oligonucleotide
ctcaacttggtcgaggatagccc

SEQ ID NO: 387 - DNA, 23 bp, Artificial Sequence
  Description of Artificial Sequence Synthetic oligonucleotide
cttggtcgaggatagcccggcca

SEQ ID NO: 388 - DNA, 23 bp, Artificial Sequence
  Description of Artificial Sequence Synthetic oligonucleotide
ttggtcgaggatagcccggccac

SEQ ID NO: 389 - DNA, 23 bp, Artificial Sequence
  Description of Artificial Sequence Synthetic oligonucleotide
ttccaatcctccggaggagtcat

SEQ ID NO: 390 - DNA, 23 bp, Artificial Sequence
  Description of Artificial Sequence Synthetic oligonucleotide
ctccggaggagtcatccccaccg

SEQ ID NO: 391 - DNA, 23 bp, Artificial Sequence
  Description of Artificial Sequence Synthetic oligonucleotide
ttaaacacatcaacaaggcaggc

SEQ ID NO: 392 - DNA, 23 bp, Artificial Sequence
  Description of Artificial Sequence Synthetic oligonucleotide
ctctatattattggagatggtcg

SEQ ID NO: 393 - DNA, 23 bp, Artificial Sequence
  Description of Artificial Sequence Synthetic oligonucleotide
ctatattattggagatggtcgcg

SEQ ID NO: 394 - DNA, 23 bp, Artificial Sequence
  Description of Artificial Sequence Synthetic oligonucleotide
ttattggagatggtcgcggcgct

SEQ ID NO: 395 - DNA, 23 bp, Artificial Sequence
  Description of Artificial Sequence Synthetic oligonucleotide
ttggagatggtcgcggcgctgtc

SEQ ID NO: 396 - DNA, 23 bp, Artificial Sequence
  Description of Artificial Sequence Synthetic oligonucleotide
ctgtccttggtcatgtagaacat

SEQ ID NO: 397 - DNA, 23 bp, Artificial Sequence
  Description of Artificial Sequence Synthetic oligonucleotide
cttggtcatgtagaacatcagac

SEQ ID NO: 398 - DNA, 23 bp, Artificial Sequence
  Description of Artificial Sequence Synthetic oligonucleotide
ttggtcatgtagaacatcagacc

SEQ ID NO: 399 - DNA, 23 bp, Artificial Sequence
  Description of Artificial Sequence Synthetic oligonucleotide
ctccctcaggtggagatggataa

SEQ ID NO: 400 - DNA, 23 bp, Artificial Sequence
  Description of Artificial Sequence Synthetic oligonucleotide
ctcaggtggagatggataacatc

SEQ ID NO: 401 - DNA, 23 bp, Artificial Sequence
  Description of Artificial Sequence Synthetic oligonucleotide
cttcactgtttgggatcacagtc

SEQ ID NO: 402 - DNA, 23 bp, Artificial Sequence
  Description of Artificial Sequence Synthetic oligonucleotide
ttcactgtttgggatcacagtct

SEQ ID NO: 403 - DNA, 23 bp, Artificial Sequence
  Description of Artificial Sequence Synthetic oligonucleotide
ctgtttgggatcacagtctgctt

SEQ ID NO: 404 - DNA, 23 bp, Artificial Sequence
  Description of Artificial Sequence Synthetic oligonucleotide
tttgggatcacagtctgctttgc

SEQ ID NO: 405 - DNA, 23 bp, Artificial Sequence
  Description of Artificial Sequence Synthetic oligonucleotide
ttgggatcacagtctgctttgcg

SEQ ID NO: 406 - DNA, 23 bp, Artificial Sequence
  Description of Artificial Sequence Synthetic oligonucleotide
ctgctttgcgcagacaatctcga

SEQ ID NO: 407 - DNA, 23 bp, Artificial Sequence
  Description of Artificial Sequence Synthetic oligonucleotide
ctttgcgcagacaatctcgaaca

SEQ ID NO: 408 - DNA, 23 bp, Artificial Sequence
  Description of Artificial Sequence Synthetic oligonucleotide
tttgcgcagacaatctcgaacat

SEQ ID NO: 409 - DNA, 23 bp, Artificial Sequence
  Description of Artificial Sequence Synthetic oligonucleotide
ttgcgcagacaatctcgaacatg

SEQ ID NO: 410 - DNA, 23 bp, Artificial Sequence
  Description of Artificial Sequence Synthetic oligonucleotide
ctcgaacatgggcttctcgacac

SEQ ID NO: 411 - DNA, 23 bp, Artificial Sequence
  Description of Artificial Sequence Synthetic oligonucleotide
cttctcgacaccgatgatggggt

SEQ ID NO: 412 - DNA, 23 bp, Artificial Sequence
  Description of Artificial Sequence Synthetic oligonucleotide
ttctcgacaccgatgatggggtc

SEQ ID NO: 413 - DNA, 23 bp, Artificial Sequence
  Description of Artificial Sequence Synthetic oligonucleotide
ctcgacaccgatgatggggtctg

SEQ ID NO: 414 - DNA, 23 bp, Artificial Sequence
  Description of Artificial Sequence Synthetic oligonucleotide
ctgacccaacaatcagcgcagca

SEQ ID NO: 415 - DNA, 23 bp, Artificial Sequence
  Description of Artificial Sequence Synthetic oligonucleotide
ctccatctccgaaaagagagttg

SEQ ID NO: 416 - DNA, 23 bp, Artificial Sequence
  Description of Artificial Sequence Synthetic oligonucleotide
ctccgaaaagagagttgccgata

SEQ ID NO: 417 - DNA, 23 bp, Artificial Sequence
  Description of Artificial Sequence Synthetic oligonucleotide
ttgccgatagcattgtcgatatg

SEQ ID NO: 418 - DNA, 23 bp, Artificial Sequence
  Description of Artificial Sequence Synthetic oligonucleotide
ttggggccacggagcgcaatgat

SEQ ID NO: 419 - DNA, 23 bp, Artificial Sequence
  Description of Artificial Sequence Synthetic oligonucleotide
ctctgcgcacaccatcagaacac

SEQ ID NO: 420 - DNA, 23 bp, Artificial Sequence
  Description of Artificial Sequence Synthetic oligonucleotide
ctgcgcacaccatcagaacacga

SEQ ID NO: 421 - DNA, 23 bp, Artificial Sequence
  Description of Artificial Sequence Synthetic oligonucleotide
ttgttctcggccagatccttggc

SEQ ID NO: 422 - DNA, 23 bp, Artificial Sequence
  Description of Artificial Sequence Synthetic oligonucleotide
ttctcggccagatccttggcata

SEQ ID NO: 423 - DNA, 23 bp, Artificial Sequence
  Description of Artificial Sequence Synthetic oligonucleotide
ctcggccagatccttggcatacc

SEQ ID NO: 424 - DNA, 23 bp, Artificial Sequence
  Description of Artificial Sequence Synthetic oligonucleotide
cttggcataccgcatgacggtgc

SEQ ID NO: 425 - DNA, 23 bp, Artificial Sequence
  Description of Artificial Sequence Synthetic oligonucleotide
ttggcataccgcatgacggtgcc

SEQ ID NO: 426 - DNA, 23 bp, Artificial Sequence
  Description of Artificial Sequence Synthetic oligonucleotide
ctgcatgtagatgcagtatttgt

SEQ ID NO: 427 - DNA, 23 bp, Artificial Sequence
  Description of Artificial Sequence Synthetic oligonucleotide
tttgttgacattggtgcggaggc

SEQ ID NO: 428 - DNA, 23 bp, Artificial Sequence
  Description of Artificial Sequence Synthetic oligonucleotide
ttgttgacattggtgcggaggcc

SEQ ID NO: 429 - DNA, 23 bp, Artificial Sequence
  Description of Artificial Sequence Synthetic oligonucleotide
ttgacattggtgcggaggccaag

SEQ ID NO: 430 - DNA, 23 bp, Artificial Sequence
  Description of Artificial Sequence Synthetic oligonucleotide
ttggtgcggaggccaaggagctt

SEQ ID NO: 431 - DNA, 23 bp, Artificial Sequence
  Description of Artificial Sequence Synthetic oligonucleotide
cttagcgcactggaaatcggcac

SEQ ID NO: 432 - DNA, 23 bp, Artificial Sequence
  Description of Artificial Sequence Synthetic oligonucleotide
ttagcgcactggaaatcggcact

SEQ ID NO: 433 - DNA, 23 bp, Artificial Sequence
  Description of Artificial Sequence Synthetic oligonucleotide
ctggaaatcggcactaggcatgt

SEQ ID NO: 434 - DNA, 23 bp, Artificial Sequence
  Description of Artificial Sequence Synthetic oligonucleotide
ctaggcatgtcaacaccgccgct

SEQ ID NO: 435 - DNA, 23 bp, Artificial Sequence
  Description of Artificial Sequence Synthetic oligonucleotide
ctggtgcagaagacgaggtgagt

SEQ ID NO: 436 - DNA, 23 bp, Artificial Sequence
  Description of Artificial Sequence Synthetic oligonucleotide
ctcagacttagggcgtccccact

SEQ ID NO: 437 - DNA, 23 bp, Artificial Sequence
  Description of Artificial Sequence Synthetic oligonucleotide
cttagggcgtccccactcctcaa

SEQ ID NO: 438 - DNA, 23 bp, Artificial Sequence
  Description of Artificial Sequence Synthetic oligonucleotide
ttagggcgtccccactcctcaat

SEQ ID NO: 439 - DNA, 23 bp, Artificial Sequence
  Description of Artificial Sequence Synthetic oligonucleotide
ctcctcaatggctttaacagccg

SEQ ID NO: 440 - DNA, 23 bp, Artificial Sequence
  Description of Artificial Sequence Synthetic oligonucleotide
ctcaatggctttaacagccgctt

SEQ ID NO: 441 - DNA, 23 bp, Artificial Sequence
  Description of Artificial Sequence Synthetic oligonucleotide
ctttaacagccgcttccgccccc

SEQ ID NO: 442 - DNA, 23 bp, Artificial Sequence
  Description of Artificial Sequence Synthetic oligonucleotide
tttaacagccgcttccgccccca

SEQ ID NO: 443 - DNA, 23 bp, Artificial Sequence
  Description of Artificial Sequence Synthetic oligonucleotide
ttaacagccgcttccgcccccag

SEQ ID NO: 444 - DNA, 23 bp, Artificial Sequence
  Description of Artificial Sequence Synthetic oligonucleotide
cttccgcccccagcgccggcacc

SEQ ID NO: 445 - DNA, 23 bp, Artificial Sequence
  Description of Artificial Sequence Synthetic oligonucleotide
ttccgcccccagcgccggcaccc

SEQ ID NO: 446 - DNA, 23 bp, Artificial Sequence
  Description of Artificial Sequence Synthetic oligonucleotide
ctgtcgttgaggctgggcttgtc

SEQ ID NO: 447 - DNA, 23 bp, Artificial Sequence
  Description of Artificial Sequence Synthetic oligonucleotide
ttgaggctgggcttgtcaaatga

SEQ ID NO: 448 - DNA, 23 bp, Artificial Sequence
  Description of Artificial Sequence Synthetic oligonucleotide
ctgggcttgtcaaatgaagtaat

SEQ ID NO: 449 - DNA, 23 bp, Artificial Sequence
  Description of Artificial Sequence Synthetic oligonucleotide
ttgggatacttcttcaagaactc

SEQ ID NO: 450 - DNA, 23 bp, Artificial Sequence
  Description of Artificial Sequence Synthetic oligonucleotide
cttcttcaagaactcctcgtcga

SEQ ID NO: 451 - DNA, 23 bp, Artificial Sequence
  Description of Artificial Sequence Synthetic oligonucleotide
ttcttcaagaactcctcgtcgaa

SEQ ID NO: 452 - DNA, 23 bp, Artificial Sequence
  Description of Artificial Sequence Synthetic oligonucleotide
cttcaagaactcctcgtcgaagt

SEQ ID NO: 453 - DNA, 23 bp, Artificial Sequence
  Description of Artificial Sequence Synthetic oligonucleotide
ttcaagaactcctcgtcgaagtt

SEQ ID NO: 454 - DNA, 23 bp, Artificial Sequence
  Description of Artificial Sequence Synthetic oligonucleotide
ctcctcgtcgaagttaaagtaac

SEQ ID NO: 455 - DNA, 23 bp, Artificial Sequence
  Description of Artificial Sequence Synthetic oligonucleotide
ctcgtcgaagttaaagtaacgct

SEQ ID NO: 456 - DNA, 23 bp, Artificial Sequence
  Description of Artificial Sequence Synthetic oligonucleotide
ttaaagtaacgcttgcctatcat

SEQ ID NO: 457 - DNA, 23 bp, Artificial Sequence
  Description of Artificial Sequence Synthetic oligonucleotide
cttgcctatcatcgttttcttgc

SEQ ID NO: 458 - DNA, 23 bp, Artificial Sequence
  Description of Artificial Sequence Synthetic oligonucleotide
ttgcctatcatcgttttcttgca

SEQ ID NO: 459 - DNA, 23 bp, Artificial Sequence
  Description of Artificial Sequence Synthetic oligonucleotide
ctatcatcgttttcttgcagatg

SEQ ID NO: 460 - DNA, 23 bp, Artificial Sequence
  Description of Artificial Sequence Synthetic oligonucleotide
ttttcttgcagatgcgatcgaac

SEQ ID NO: 461 - DNA, 23 bp, Artificial Sequence
  Description of Artificial Sequence Synthetic oligonucleotide
tttcttgcagatgcgatcgaact

SEQ ID NO: 462 - DNA, 23 bp, Artificial Sequence
  Description of Artificial Sequence Synthetic oligonucleotide
ttcttgcagatgcgatcgaactt

SEQ ID NO: 463 - DNA, 23 bp, Artificial Sequence
  Description of Artificial Sequence Synthetic oligonucleotide
cttgcagatgcgatcgaacttct

SEQ ID NO: 464 - DNA, 23 bp, Artificial Sequence
  Description of Artificial Sequence Synthetic oligonucleotide
ttgcagatgcgatcgaacttctt

SEQ ID NO: 465 - DNA, 23 bp, Artificial Sequence
  Description of Artificial Sequence Synthetic oligonucleotide
cttcttcttgagctcgaccttgt

SEQ ID NO: 466 - DNA, 23 bp, Artificial Sequence
  Description of Artificial Sequence Synthetic oligonucleotide
ttcttcttgagctcgaccttgtg

SEQ ID NO: 467 - DNA, 23 bp, Artificial Sequence
  Description of Artificial Sequence Synthetic oligonucleotide
cttcttgagctcgaccttgtgct

SEQ ID NO: 468 - DNA, 23 bp, Artificial Sequence
  Description of Artificial Sequence Synthetic oligonucleotide
ttcttgagctcgaccttgtgctc

SEQ ID NO: 469 - DNA, 23 bp, Artificial Sequence
  Description of Artificial Sequence Synthetic oligonucleotide
cttgagctcgaccttgtgctcgc

SEQ ID NO: 470 - DNA, 23 bp, Artificial Sequence
  Description of Artificial Sequence Synthetic oligonucleotide
ttgagctcgaccttgtgctcgct

SEQ ID NO: 471 - DNA, 23 bp, Artificial Sequence
  Description of Artificial Sequence Synthetic oligonucleotide
cttgtgctcgctgttggtgaccc

SEQ ID NO: 472 - DNA, 23 bp, Artificial Sequence
  Description of Artificial Sequence Synthetic oligonucleotide
ttgtgctcgctgttggtgacccg

SEQ ID NO: 473 - DNA, 23 bp, Artificial Sequence
  Description of Artificial Sequence Synthetic oligonucleotide
ctcgctgttggtgacccggaagt

SEQ ID NO: 474 - DNA, 23 bp, Artificial Sequence
  Description of Artificial Sequence Synthetic oligonucleotide
ctgttggtgacccggaagtagaa

SEQ ID NO: 475 - DNA, 23 bp, Artificial Sequence
  Description of Artificial Sequence Synthetic oligonucleotide
ttggtgacccggaagtagaaatc

SEQ ID NO: 476 - DNA, 23 bp, Artificial Sequence
  Description of Artificial Sequence Synthetic oligonucleotide
ctgcgaaatgatatgaggagggt

SEQ ID NO: 477 - DNA, 23 bp, Artificial Sequence
  Description of Artificial Sequence Synthetic oligonucleotide
ctgttccaatggccatcacggtc

SEQ ID NO: 478 - DNA, 23 bp, Artificial Sequence
  Description of Artificial Sequence Synthetic oligonucleotide
ttccaatggccatcacggtcgca

SEQ ID NO: 479 - DNA, 23 bp, Artificial Sequence
  Description of Artificial Sequence Synthetic oligonucleotide
ttccgtcggccttttgggccttt

SEQ ID NO: 480 - DNA, 23 bp, Artificial Sequence
  Description of Artificial Sequence Synthetic oligonucleotide
cttttgggcctttcggatgccct

SEQ ID NO: 481 - DNA, 23 bp, Artificial Sequence
  Description of Artificial Sequence Synthetic oligonucleotide
ttttgggcctttcggatgccctg

SEQ ID NO: 482 - DNA, 23 bp, Artificial Sequence
  Description of Artificial Sequence Synthetic oligonucleotide
tttgggcctttcggatgccctgc

SEQ ID NO: 483 - DNA, 23 bp, Artificial Sequence
  Description of Artificial Sequence Synthetic oligonucleotide
ttgggcctttcggatgccctgca

SEQ ID NO: 484 - DNA, 23 bp, Artificial Sequence
  Description of Artificial Sequence Synthetic oligonucleotide
ctttcggatgccctgcacatcct

SEQ ID NO: 485 - DNA, 23 bp, Artificial Sequence
  Description of Artificial Sequence Synthetic oligonucleotide
tttcggatgccctgcacatcctt

SEQ ID NO: 486 - DNA, 23 bp, Artificial Sequence
  Description of Artificial Sequence Synthetic oligonucleotide
ttcggatgccctgcacatccttc

SEQ ID NO: 487 - DNA, 23 bp, Artificial Sequence
  Description of Artificial Sequence Synthetic oligonucleotide
ctgcacatccttcatcaaaggcg

SEQ ID NO: 488 - DNA, 23 bp, Artificial Sequence
  Description of Artificial Sequence Synthetic oligonucleotide
ttcatcaaaggcgtagagtcgga

SEQ ID NO: 489 - DNA, 26 bp, Artificial Sequence
  Description of Artificial Sequence Synthetic oligonucleotide
ccgactctacgcctttgatgaaggat

SEQ ID NO: 490 - DNA, 26 bp, Artificial Sequence
  Description of Artificial Sequence Synthetic oligonucleotide
tcatttcgcaggacagctacgcggat

SEQ ID NO: 491 - DNA, 26 bp, Artificial Sequence
  Description of Artificial Sequence Synthetic oligonucleotide
agctacgcggatttctacttccgggt

SEQ ID NO: 492 - DNA, 26 bp, Artificial Sequence
  Description of Artificial Sequence Synthetic oligonucleotide
gcgttactttaacttcgacgaggagt

SEQ ID NO: 493 - DNA, 26 bp, Artificial Sequence
  Description of Artificial Sequence Synthetic oligonucleotide
aggcatgacatttgcattcctggggt

SEQ ID NO: 494 - DNA, 26 bp, Artificial Sequence
  Description of Artificial Sequence Synthetic oligonucleotide
agcggctgttaaagccattgaggagt

SEQ ID NO: 495 - DNA, 26 bp, Artificial Sequence
  Description of Artificial Sequence Synthetic oligonucleotide
caaatactgcatctacatgcagggat

SEQ ID NO: 496 - DNA, 26 bp, Artificial Sequence
  Description of Artificial Sequence Synthetic oligonucleotide
gcaccgtcatgcggtatgccaaggat

SEQ ID NO: 497 - DNA, 26 bp, Artificial Sequence
  Description of Artificial Sequence Synthetic oligonucleotide
tggagctgctgcgctgattgttgggt

SEQ ID NO: 498 - DNA, 26 bp, Artificial Sequence
  Description of Artificial Sequence Synthetic oligonucleotide
tgttatccatctccacctgagggagt

SEQ ID NO: 499 - DNA, 26 bp, Artificial Sequence
  Description of Artificial Sequence Synthetic oligonucleotide
tccatctccacctgagggagtcgggt

SEQ ID NO: 500 - DNA, 26 bp, Artificial Sequence
  Description of Artificial Sequence Synthetic oligonucleotide
gttgatgtgtttaagtcggtggggat

SEQ ID NO: 501 - DNA, 26 bp, Artificial Sequence
  Description of Artificial Sequence Synthetic oligonucleotide
cggtggggatgactcctccggaggat

SEQ ID NO: 502 - DNA, 26 bp, Artificial Sequence
  Description of Artificial Sequence Synthetic oligonucleotide
gaggattggaactctctcttctggat

SEQ ID NO: 503 - DNA, 26 bp, Artificial Sequence
  Description of Artificial Sequence Synthetic oligonucleotide
gtgcgactcgaacagttctctgggat

SEQ ID NO: 504 - DNA, 26 bp, Artificial Sequence
  Description of Artificial Sequence Synthetic oligonucleotide
gtgcatgtgtgctctttatactggat

SEQ ID NO: 505 - DNA, 26 bp, Artificial Sequence
  Description of Artificial Sequence Synthetic oligonucleotide
gagaagaaaatctgctgctgaaggat

SEQ ID NO: 506 - DNA, 26 bp, Artificial Sequence
  Description of Artificial Sequence Synthetic oligonucleotide
aggattagaaacctacggagaggggt

SEQ ID NO: 507 - DNA, 26 bp, Artificial Sequence
  Description of Artificial Sequence Synthetic oligonucleotide
agaaacctacggagaggggttggagt

SEQ ID NO: 508 - DNA, 26 bp, Artificial Sequence
  Description of Artificial Sequence Synthetic oligonucleotide
cctacggagaggggttggagtggggt

SEQ ID NO: 509 - DNA, 26 bp, Artificial Sequence
  Description of Artificial Sequence Synthetic oligonucleotide
gtcttgcttggttttggaccaggaat

SEQ ID NO: 510 - DNA, 26 bp, Artificial Sequence
  Description of Artificial Sequence Synthetic oligonucleotide
acaggaggcaggctatgaagaaggat

SEQ ID NO: 511 - DNA, 26 bp, Artificial Sequence
  Description of Artificial Sequence Synthetic oligonucleotide
ccataatcccagagaactgttcgagt

SEQ ID NO: 512 - DNA, 26 bp, Artificial Sequence
  Description of Artificial Sequence Synthetic oligonucleotide
agcttggcctcaacttggtcgaggat

SEQ ID NO: 513 - DNA, 26 bp, Artificial Sequence
  Description of Artificial Sequence Synthetic oligonucleotide
gtcgaggatagcccggccaccgggat

SEQ ID NO: 514 - DNA, 26 bp, Artificial Sequence
  Description of Artificial Sequence Synthetic oligonucleotide
atagcccggccaccgggatgagggat

SEQ ID NO: 515 - DNA, 26 bp, Artificial Sequence
  Description of Artificial Sequence Synthetic oligonucleotide
gggatgagggatccagaagagagagt

SEQ ID NO: 516 - DNA, 26 bp, Artificial Sequence
  Description of Artificial Sequence Synthetic oligonucleotide
agagagttccaatcctccggaggagt

SEQ ID NO: 517 - DNA, 26 bp, Artificial Sequence
  Description of Artificial Sequence Synthetic oligonucleotide
cccgactccctcaggtggagatggat

SEQ ID NO: 518 - DNA, 26 bp, Artificial Sequence
  Description of Artificial Sequence Synthetic oligonucleotide
tggataacatcttcactgtttgggat

SEQ ID NO: 519 - DNA, 26 bp, Artificial Sequence
  Description of Artificial Sequence Synthetic oligonucleotide
gggcttctcgacaccgatgatggggt

SEQ ID NO: 520 - DNA, 26 bp, Artificial Sequence
  Description of Artificial Sequence Synthetic oligonucleotide
agcagctccatctccgaaaagagagt

SEQ ID NO: 521 - DNA, 26 bp, Artificial Sequence
  Description of Artificial Sequence Synthetic oligonucleotide
gccgatagcattgtcgatatgggaat

SEQ ID NO: 522 - DNA, 26 bp, Artificial Sequence
  Description of Artificial Sequence Synthetic oligonucleotide
ccgctggtgcagaagacgaggtgagt

SEQ ID NO: 523 - DNA, 26 bp, Artificial Sequence
  Description of Artificial Sequence Synthetic oligonucleotide
gcccccagcgccggcaccccaggaat

SEQ ID NO: 524 - DNA, 26 bp, Artificial Sequence
  Description of Artificial Sequence Synthetic oligonucleotide
cttgtcaaatgaagtaatgttgggat

SEQ ID NO: 525 - DNA, 26 bp, Artificial Sequence
  Description of Artificial Sequence Synthetic oligonucleotide
gtcctgcgaaatgatatgaggagggt

SEQ ID NO: 526 - DNA, 26 bp, Artificial Sequence
  Description of Artificial Sequence Synthetic oligonucleotide
ccgtcggccttttgggcctttcggat

SEQ ID NO: 527 - DNA, 26 bp, Artificial Sequence
  Description of Artificial Sequence Synthetic oligonucleotide
cacatccttcatcaaaggcgtagagt

SEQ ID NO: 528 - DNA, 26 bp, Artificial Sequence
  Description of Artificial Sequence Synthetic oligonucleotide
tcatcaaaggcgtagagtcggagagt

SEQ ID NO: 529 - DNA, 27 bp, Artificial Sequence
  Description of Artificial Sequence Synthetic oligonucleotide
tcatttcgcaggacagctacgcggatt

SEQ ID NO: 530 - DNA, 27 bp, Artificial Sequence
  Description of Artificial Sequence Synthetic oligonucleotide
gcggtgttgacatgcctagtgccgatt

SEQ ID NO: 531 - DNA, 27 bp, Artificial Sequence
  Description of Artificial Sequence Synthetic oligonucleotide
tcattgcgctccgtggccccaatgatt

SEQ ID NO: 532 - DNA, 27 bp, Artificial Sequence
  Description of Artificial Sequence Synthetic oligonucleotide
ttcggagatggagctgctgcgctgatt

SEQ ID NO: 533 - DNA, 27 bp, Artificial Sequence
  Description of Artificial Sequence Synthetic oligonucleotide
ggtgtcgagaagcccatgttcgagatt

SEQ ID NO: 534 - DNA, 27 bp, Artificial Sequence
  Description of Artificial Sequence Synthetic oligonucleotide
cggtggggatgactcctccggaggatt

SEQ ID NO: 535 - DNA, 27 bp, Artificial Sequence
  Description of Artificial Sequence Synthetic oligonucleotide
gtgcgactcgaacagttctctgggatt

SEQ ID NO: 536 - DNA, 27 bp, Artificial Sequence
  Description of Artificial Sequence Synthetic oligonucleotide
gagaagaaaatctgctgctgaaggatt

SEQ ID NO: 537 - DNA, 27 bp, Artificial Sequence
  Description of Artificial Sequence Synthetic oligonucleotide
aggtttctaatccttcagcagcagatt

SEQ ID NO: 538 - DNA, 27 bp, Artificial Sequence
  Description of Artificial Sequence Synthetic oligonucleotide
aagaagttcgatcgcatctgcaagaaa

SEQ ID NO: 539 - DNA, 27 bp, Artificial Sequence
  Description of Artificial Sequence Synthetic oligonucleotide
ctttatactggatgaaatgagaagaaa

SEQ ID NO: 540 - DNA, 27 bp, Artificial Sequence
  Description of Artificial Sequence Synthetic oligonucleotide
gaaaatctgctgctgaaggattagaaa

SEQ ID NO: 541 - DNA, 27 bp, Artificial Sequence
  Description of Artificial Sequence Synthetic oligonucleotide
tcgctgttggtgacccggaagtagaaa

SEQ ID NO: 542 - DNA, 26 bp, Artificial Sequence
  Description of Artificial Sequence Synthetic oligonucleotide
aagttcgatcgcatctgcaagaaaac

SEQ ID NO: 543 - DNA, 26 bp, Artificial Sequence
  Description of Artificial Sequence Synthetic oligonucleotide
ttcaatagtcattcctggtccaaaac

SEQ ID NO: 544 - DNA, 28 bp, Artificial Sequence
  Description of Artificial Sequence Synthetic oligonucleotide
ctacttccgggtcaccaacagcgagcac

SEQ ID NO: 545 - DNA, 28 bp, Artificial Sequence
  Description of Artificial Sequence Synthetic oligonucleotide
ctgagatcactcacctcgtcttctgcac

SEQ ID NO: 546 - DNA, 28 bp, Artificial Sequence
  Description of Artificial Sequence Synthetic oligonucleotide
agtgcgctaagctccttggcctccgcac

SEQ ID NO: 547 - DNA, 28 bp, Artificial Sequence
  Description of Artificial Sequence Synthetic oligonucleotide
tggcctccgcaccaatgtcaacaaatac

SEQ ID NO: 548 - DNA, 28 bp, Artificial Sequence
  Description of Artificial Sequence Synthetic oligonucleotide
acatgcagggatgctatgctggtggcac

SEQ ID NO: 549 - DNA, 28 bp, Artificial Sequence
  Description of Artificial Sequence Synthetic oligonucleotide
atgataagtgcatgtgtgctctttatac

SEQ ID NO: 550 - DNA, 28 bp, Artificial Sequence
  Description of Artificial Sequence Synthetic oligonucleotide
attcctggtccaaaaccaagcaagacac

SEQ ID NO: 551 - DNA, 28 bp, Artificial Sequence
  Description of Artificial Sequence Synthetic oligonucleotide
ttctcatttcatccagtataaagagcac

SEQ ID NO: 552 - DNA, 28 bp, Artificial Sequence
  Description of Artificial Sequence Synthetic oligonucleotide
ctcatttcatccagtataaagagcacac

SEQ ID NO: 553 - DNA, 28 bp, Artificial Sequence
  Description of Artificial Sequence Synthetic oligonucleotide
tcatccagtataaagagcacacatgcac

SEQ ID NO: 554 - DNA, 28 bp, Artificial Sequence
  Description of Artificial Sequence Synthetic oligonucleotide
taatcccagagaactgttcgagtcgcac

SEQ ID NO: 555 - DNA, 28 bp, Artificial Sequence
  Description of Artificial Sequence Synthetic oligonucleotide
gaggagtcatccccaccgacttaaacac

SEQ ID NO: 556 - DNA, 28 bp, Artificial Sequence
  Description of Artificial Sequence Synthetic oligonucleotide
acaatctcgaacatgggcttctcgacac

SEQ ID NO: 557 - DNA, 28 bp, Artificial Sequence
  Description of Artificial Sequence Synthetic oligonucleotide
gagcgcaatgatggtgagctctgcgcac

SEQ ID NO: 558 - DNA, 28 bp, Artificial Sequence
  Description of Artificial Sequence Synthetic oligonucleotide
gcgcaatgatggtgagctctgcgcacac

SEQ ID NO: 559 - DNA, 28 bp, Artificial Sequence
  Description of Artificial Sequence Synthetic oligonucleotide
gtgagctctgcgcacaccatcagaacac

SEQ ID NO: 560 - DNA, 28 bp, Artificial Sequence
  Description of Artificial Sequence Synthetic oligonucleotide
tctgcgcacaccatcagaacacgagcac

SEQ ID NO: 561 - DNA, 28 bp, Artificial Sequence
  Description of Artificial Sequence Synthetic oligonucleotide
gttgttctcggccagatccttggcatac

SEQ ID NO: 562 - DNA, 28 bp, Artificial Sequence
  Description of Artificial Sequence Synthetic oligonucleotide
ggtgcggaggccaaggagcttagcgcac

SEQ ID NO: 563 - DNA, 28 bp, Artificial Sequence
  Description of Artificial Sequence Synthetic oligonucleotide
aggagcttagcgcactggaaatcggcac

SEQ ID NO: 564 - DNA, 28 bp, Artificial Sequence
  Description of Artificial Sequence Synthetic oligonucleotide
tggaaatcggcactaggcatgtcaacac

SEQ ID NO: 565 - DNA, 28 bp, Artificial Sequence
  Description of Artificial Sequence Synthetic oligonucleotide
cagccgcttccgcccccagcgccggcac

SEQ ID NO: 566 - DNA, 28 bp, Artificial Sequence
  Description of Artificial Sequence Synthetic oligonucleotide
cttgtcaaatgaagtaatgttgggatac

SEQ ID NO: 567 - DNA, 28 bp, Artificial Sequence
  Description of Artificial Sequence Synthetic oligonucleotide
ccttttgggcctttcggatgccctgcac

SEQ ID NO: 568 - DNA, 23 bp, Artificial Sequence
  Description of Artificial Sequence Synthetic oligonucleotide
ctccaactactcgccagtgatgg

SEQ ID NO: 569 - DNA, 23 bp, Artificial Sequence
  Description of Artificial Sequence Synthetic oligonucleotide
caactactcgccagtgatggagg

SEQ ID NO: 570 - DNA, 23 bp, Artificial Sequence
  Description of Artificial Sequence Synthetic oligonucleotide
gccagtgatggaggatgtgcagg

SEQ ID NO: 571 - DNA, 23 bp, Artificial Sequence
  Description of Artificial Sequence Synthetic oligonucleotide
ggatgtgcaggccatccgaaagg

SEQ ID NO: 572 - DNA, 23 bp, Artificial Sequence
  Description of Artificial Sequence Synthetic oligonucleotide
cgaaaggcccaaaaagcagatgg

SEQ ID NO: 573 - DNA, 23 bp, Artificial Sequence
  Description of Artificial Sequence Synthetic oligonucleotide
agatggaaccgcaacagtgatgg

SEQ ID NO: 574 - DNA, 23 bp, Artificial Sequence
  Description of Artificial Sequence Synthetic oligonucleotide
accgcaacagtgatggccatcgg

SEQ ID NO: 575 - DNA, 23 bp, Artificial Sequence
  Description of Artificial Sequence Synthetic oligonucleotide
ccctcctcatatctttcctcagg

SEQ ID NO: 576 - DNA, 23 bp, Artificial Sequence
  Description of Artificial Sequence Synthetic oligonucleotide
ctttcctcaggacacctatgcgg

SEQ ID NO: 577 - DNA, 23 bp, Artificial Sequence
  Description of Artificial Sequence Synthetic oligonucleotide
atctgcaaaaagacaatgatagg

SEQ ID NO: 578 - DNA, 23 bp, Artificial Sequence
  Description of Artificial Sequence Synthetic oligonucleotide
gcgctacttcaactacgatgagg

SEQ ID NO: 579 - DNA, 23 bp, Artificial Sequence
  Description of Artificial Sequence Synthetic oligonucleotide
gccgagcctcaacgaccgccagg

SEQ ID NO: 580 - DNA, 23 bp, Artificial Sequence
  Description of Artificial Sequence Synthetic oligonucleotide
cgccaggatatttgcgtccctgg

SEQ ID NO: 581 - DNA, 23 bp, Artificial Sequence
  Description of Artificial Sequence Synthetic oligonucleotide
gccaggatatttgcgtccctggg

SEQ ID NO: 582 - DNA, 23 bp, Artificial Sequence
  Description of Artificial Sequence Synthetic oligonucleotide
ccaggatatttgcgtccctgggg

SEQ ID NO: 583 - DNA, 23 bp, Artificial Sequence
  Description of Artificial Sequence Synthetic oligonucleotide
tatttgcgtccctggggtgccgg

SEQ ID NO: 584 - DNA, 23 bp, Artificial Sequence
  Description of Artificial Sequence Synthetic oligonucleotide
cgtccctggggtgccggccctgg

SEQ ID NO: 585 - DNA, 23 bp, Artificial Sequence
  Description of Artificial Sequence Synthetic oligonucleotide
gtccctggggtgccggccctggg

SEQ ID NO: 586 - DNA, 23 bp, Artificial Sequence
  Description of Artificial Sequence Synthetic oligonucleotide
ctgtcaaagccatcgctgaatgg

SEQ ID NO: 587 - DNA, 23 bp, Artificial Sequence
  Description of Artificial Sequence Synthetic oligonucleotide
tgtcaaagccatcgctgaatggg

SEQ ID NO: 588 - DNA, 23 bp, Artificial Sequence
  Description of Artificial Sequence Synthetic oligonucleotide
gtcaaagccatcgctgaatgggg

SEQ ID NO: 589 - DNA, 23 bp, Artificial Sequence
  Description of Artificial Sequence Synthetic oligonucleotide
ctcgtcttctgcacctcctgcgg

SEQ ID NO: 590 - DNA, 23 bp, Artificial Sequence
  Description of Artificial Sequence Synthetic oligonucleotide
ttccagtgcgccaagctcctcgg

SEQ ID NO: 591 - DNA, 23 bp, Artificial Sequence
  Description of Artificial Sequence Synthetic oligonucleotide
caagtattgcgtctacatgcagg

SEQ ID NO: 592 - DNA, 23 bp, Artificial Sequence
  Description of Artificial Sequence Synthetic oligonucleotide
aagtattgcgtctacatgcaggg

SEQ ID NO: 593 - DNA, 23 bp, Artificial Sequence
  Description of Artificial Sequence Synthetic oligonucleotide
tacatgcagggatgctacgctgg

SEQ ID NO: 594 - DNA, 23 bp, Artificial Sequence
  Description of Artificial Sequence Synthetic oligonucleotide
atgcagggatgctacgctggcgg

SEQ ID NO: 595 - DNA, 23 bp, Artificial Sequence
  Description of Artificial Sequence Synthetic oligonucleotide
acgctggcggcacagtcatgcgg

SEQ ID NO: 596 - DNA, 23 bp, Artificial Sequence
  Description of Artificial Sequence Synthetic oligonucleotide
cacagtcatgcggtacgccaagg

SEQ ID NO: 597 - DNA, 23 bp, Artificial Sequence
  Description of Artificial Sequence Synthetic oligonucleotide
gatctcgccgagaacaaccgcgg

SEQ ID NO: 598 - DNA, 23 bp, Artificial Sequence
  Description of Artificial Sequence Synthetic oligonucleotide
ccgcggtgctcgtgttctagtgg

SEQ ID NO: 599 - DNA, 23 bp, Artificial Sequence
  Description of Artificial Sequence Synthetic oligonucleotide
tgcgccgagctcaccatcatcgg

SEQ ID NO: 600 - DNA, 23 bp, Artificial Sequence
  Description of Artificial Sequence Synthetic oligonucleotide
gcgccgagctcaccatcatcggg

SEQ ID NO: 601 - DNA, 23 bp, Artificial Sequence
  Description of Artificial Sequence Synthetic oligonucleotide
ctcaccatcatcgggcttcgcgg

SEQ ID NO: 602 - DNA, 23 bp, Artificial Sequence
  Description of Artificial Sequence Synthetic oligonucleotide
tcccatctcgacaacgccatcgg

SEQ ID NO: 603 - DNA, 23 bp, Artificial Sequence
  Description of Artificial Sequence Synthetic oligonucleotide
gccatcggcaactccctttttgg

SEQ ID NO: 604 - DNA, 23 bp, Artificial Sequence
  Description of Artificial Sequence Synthetic oligonucleotide
ggcaactccctttttggagacgg

SEQ ID NO: 605 - DNA, 23 bp, Artificial Sequence
  Description of Artificial Sequence Synthetic oligonucleotide
ggagctgctgcgctgattgtcgg

SEQ ID NO: 606 - DNA, 23 bp, Artificial Sequence
  Description of Artificial Sequence Synthetic oligonucleotide
gagctgctgcgctgattgtcggg

SEQ ID NO: 607 - DNA, 23 bp, Artificial Sequence
  Description of Artificial Sequence Synthetic oligonucleotide
gtcgggtcagaccccatcattgg

SEQ ID NO: 608 - DNA, 23 bp, Artificial Sequence
  Description of Artificial Sequence Synthetic oligonucleotide
tatccatctccacatgagagagg

SEQ ID NO: 609 - DNA, 23 bp, Artificial Sequence
  Description of Artificial Sequence Synthetic oligonucleotide
catctccacatgagagaggcagg

SEQ ID NO: 610 - DNA, 23 bp, Artificial Sequence
  Description of Artificial Sequence Synthetic oligonucleotide
tctaatgttctacatgagcaagg

SEQ ID NO: 611 - DNA, 23 bp, Artificial Sequence
  Description of Artificial Sequence Synthetic oligonucleotide
gaccatctccaacaatgtagagg

SEQ ID NO: 612 - DNA, 23 bp, Artificial Sequence
  Description of Artificial Sequence Synthetic oligonucleotide
ccttgtcgatgtgttcaagtcgg

SEQ ID NO: 613 - DNA, 23 bp, Artificial Sequence
  Description of Artificial Sequence Synthetic oligonucleotide
tgtcgatgtgttcaagtcggtgg

SEQ ID NO: 614 - DNA, 23 bp, Artificial Sequence
  Description of Artificial Sequence Synthetic oligonucleotide
gtcgatgtgttcaagtcggtggg

SEQ ID NO: 615 - DNA, 23 bp, Artificial Sequence
  Description of Artificial Sequence Synthetic oligonucleotide
tcgatgtgttcaagtcggtgggg

SEQ ID NO: 616 - DNA, 23 bp, Artificial Sequence
  Description of Artificial Sequence Synthetic oligonucleotide
ggtggggatgactcctcccgagg

SEQ ID NO: 617 - DNA, 23 bp, Artificial Sequence
  Description of Artificial Sequence Synthetic oligonucleotide
ggatgactcctcccgaggactgg

SEQ ID NO: 618 - DNA, 23 bp, Artificial Sequence
  Description of Artificial Sequence Synthetic oligonucleotide
aggactggaactctctcttctgg

SEQ ID NO: 619 - DNA, 23 bp, Artificial Sequence
  Description of Artificial Sequence Synthetic oligonucleotide
ctcttctggatccctcaccccgg

SEQ ID NO: 620 - DNA, 23 bp, Artificial Sequence
  Description of Artificial Sequence Synthetic oligonucleotide
ttctggatccctcaccccggcgg

SEQ ID NO: 621 - DNA, 23 bp, Artificial Sequence
  Description of Artificial Sequence Synthetic oligonucleotide
cggccgagctatcctcgaccagg

SEQ ID NO: 622 - DNA, 23 bp, Artificial Sequence
  Description of Artificial Sequence Synthetic oligonucleotide
tcctcgaccaggttgagtccagg

SEQ ID NO: 623 - DNA, 23 bp, Artificial Sequence
  Description of Artificial Sequence Synthetic oligonucleotide
aagcttcgtcccgagaagttcgg

SEQ ID NO: 624 - DNA, 23 bp, Artificial Sequence
  Description of Artificial Sequence Synthetic oligonucleotide
gcgcgactcgaactgtgctctgg

SEQ ID NO: 625 - DNA, 23 bp, Artificial Sequence
  Description of Artificial Sequence Synthetic oligonucleotide
cgcgactcgaactgtgctctggg

SEQ ID NO: 626 - DNA, 23 bp, Artificial Sequence
  Description of Artificial Sequence Synthetic oligonucleotide
cgaactgtgctctgggattgcgg

SEQ ID NO: 627 - DNA, 23 bp, Artificial Sequence
  Description of Artificial Sequence Synthetic oligonucleotide
gctctgggattgcggaaacatgg

SEQ ID NO: 628 - DNA, 23 bp, Artificial Sequence
  Description of Artificial Sequence Synthetic oligonucleotide
cgcgtgtgttctctacatattgg

SEQ ID NO: 629 - DNA, 23 bp, Artificial Sequence
  Description of Artificial Sequence Synthetic oligonucleotide
agaagaaaatctgctgctgacgg

SEQ ID NO: 630 - DNA, 23 bp, Artificial Sequence
  Description of Artificial Sequence Synthetic oligonucleotide
gctgacggactagcaacctacgg

SEQ ID NO: 631 - DNA, 23 bp, Artificial Sequence
  Description of Artificial Sequence Synthetic oligonucleotide
cggactagcaacctacggagagg

SEQ ID NO: 632 - DNA, 23 bp, Artificial Sequence
  Description of Artificial Sequence Synthetic oligonucleotide
ggactagcaacctacggagaggg

SEQ ID NO: 633 - DNA, 23 bp, Artificial Sequence
  Description of Artificial Sequence Synthetic oligonucleotide
gactagcaacctacggagagggg

SEQ ID NO: 634 - DNA, 23 bp, Artificial Sequence
  Description of Artificial Sequence Synthetic oligonucleotide
agcaacctacggagaggggctgg

SEQ ID NO: 635 - DNA, 23 bp, Artificial Sequence
  Description of Artificial Sequence Synthetic oligonucleotide
cctacggagaggggctggagtgg

SEQ ID NO: 636 - DNA, 23 bp, Artificial Sequence
  Description of Artificial Sequence Synthetic oligonucleotide
ctacggagaggggctggagtggg

SEQ ID NO: 637 - DNA, 23 bp, Artificial Sequence
  Description of Artificial Sequence Synthetic oligonucleotide
tacggagaggggctggagtgggg

SEQ ID NO: 638 - DNA, 23 bp, Artificial Sequence
  Description of Artificial Sequence Synthetic oligonucleotide
ctggagtggggtgtcttgcttgg

SEQ ID NO: 639 - DNA, 23 bp, Artificial Sequence
  Description of Artificial Sequence Synthetic oligonucleotide
tggggtgtcttgcttggtttcgg

SEQ ID NO: 640 - DNA, 23 bp, Artificial Sequence
  Description of Artificial Sequence Synthetic oligonucleotide
gtcttgcttggtttcggaccagg

SEQ ID NO: 641 - DNA, 23 bp, Artificial Sequence
  Description of Artificial Sequence Synthetic oligonucleotide
tcttgcttggtttcggaccaggg

SEQ ID NO: 642 - DNA, 23 bp, Artificial Sequence
  Description of Artificial Sequence Synthetic oligonucleotide
cagtgggcaggctgtggagaagg

SEQ ID NO: 643 - DNA, 23 bp, Artificial Sequence
  Description of Artificial Sequence Synthetic oligonucleotide
gtggagaaggatagtttcaacgg

SEQ ID NO: 644 - DNA, 23 bp, Artificial Sequence
  Description of Artificial Sequence Synthetic oligonucleotide
atagtttcaacggtcatccctgg

SEQ ID NO: 645 - DNA, 23 bp, Artificial Sequence
  Description of Artificial Sequence Synthetic oligonucleotide
ccactccagcccctctccgtagg

SEQ ID NO: 646 - DNA, 23 bp, Artificial Sequence
  Description of Artificial Sequence Synthetic oligonucleotide
tcgagtcgcgccgaacttctcgg

SEQ ID NO: 647 - DNA, 23 bp, Artificial Sequence
  Description of Artificial Sequence Synthetic oligonucleotide
cgagtcgcgccgaacttctcggg

SEQ ID NO: 648 - DNA, 23 bp, Artificial Sequence
  Description of Artificial Sequence Synthetic oligonucleotide
ctcgggacgaagcttaagcctgg

SEQ ID NO: 649 - DNA, 23 bp, Artificial Sequence
  Description of Artificial Sequence Synthetic oligonucleotide
gcttaagcctggactcaacctgg

SEQ ID NO: 650 - DNA, 23 bp, Artificial Sequence
  Description of Artificial Sequence Synthetic oligonucleotide
gcctggactcaacctggtcgagg

SEQ ID NO: 651 - DNA, 23 bp, Artificial Sequence
  Description of Artificial Sequence Synthetic oligonucleotide
caacctggtcgaggatagctcgg

SEQ ID NO: 652 - DNA, 23 bp, Artificial Sequence
  Description of Artificial Sequence Synthetic oligonucleotide
gtcgaggatagctcggccgccgg

SEQ ID NO: 653 - DNA, 23 bp, Artificial Sequence
  Description of Artificial Sequence Synthetic oligonucleotide
tcgaggatagctcggccgccggg

SEQ ID NO: 654 - DNA, 23 bp, Artificial Sequence
  Description of Artificial Sequence Synthetic oligonucleotide
cgaggatagctcggccgccgggg

SEQ ID NO: 655 - DNA, 23 bp, Artificial Sequence
  Description of Artificial Sequence Synthetic oligonucleotide
atagctcggccgccggggtgagg

SEQ ID NO: 656 - DNA, 23 bp, Artificial Sequence
  Description of Artificial Sequence Synthetic oligonucleotide
tagctcggccgccggggtgaggg

SEQ ID NO: 657 - DNA, 23 bp, Artificial Sequence
  Description of Artificial Sequence Synthetic oligonucleotide
gaagagagagttccagtcctcgg

SEQ ID NO: 658 - DNA, 23 bp, Artificial Sequence
  Description of Artificial Sequence Synthetic oligonucleotide
aagagagagttccagtcctcggg

SEQ ID NO: 659 - DNA, 23 bp, Artificial Sequence
  Description of Artificial Sequence Synthetic oligonucleotide
agagagttccagtcctcgggagg

SEQ ID NO: 660 - DNA, 23 bp, Artificial Sequence
  Description of Artificial Sequence Synthetic oligonucleotide
ccgacttgaacacatcgacaagg

SEQ ID NO: 661 - DNA, 23 bp, Artificial Sequence
  Description of Artificial Sequence Synthetic oligonucleotide
aaggcatgcctctacattgttgg

SEQ ID NO: 662 - DNA, 23 bp, Artificial Sequence
  Description of Artificial Sequence Synthetic oligonucleotide
tgcctctacattgttggagatgg

SEQ ID NO: 663 - DNA, 23 bp, Artificial Sequence
  Description of Artificial Sequence Synthetic oligonucleotide
acattgttggagatggtctcagg

SEQ ID NO: 664 - DNA, 23 bp, Artificial Sequence
  Description of Artificial Sequence Synthetic oligonucleotide
cattgttggagatggtctcaggg

SEQ ID NO: 665 - DNA, 23 bp, Artificial Sequence
  Description of Artificial Sequence Synthetic oligonucleotide
ttagacctgcctctctcatgtgg

SEQ ID NO: 666 - DNA, 23 bp, Artificial Sequence
  Description of Artificial Sequence Synthetic oligonucleotide
ctgcctctctcatgtggagatgg

SEQ ID NO: 667 - DNA, 23 bp, Artificial Sequence
  Description of Artificial Sequence Synthetic oligonucleotide
atcttcactgtttgggatcacgg

SEQ ID NO: 668 - DNA, 23 bp, Artificial Sequence
  Description of Artificial Sequence Synthetic oligonucleotide
gcgcacacgatctcgaacatagg

SEQ ID NO: 669 - DNA, 23 bp, Artificial Sequence
  Description of Artificial Sequence Synthetic oligonucleotide
aggcttctcgacgccaatgatgg

SEQ ID NO: 670 - DNA, 23 bp, Artificial Sequence
  Description of Artificial Sequence Synthetic oligonucleotide
ggcttctcgacgccaatgatggg

SEQ ID NO: 671 - DNA, 23 bp, Artificial Sequence
  Description of Artificial Sequence Synthetic oligonucleotide
gcttctcgacgccaatgatgggg

SEQ ID NO: 672 - DNA, 23 bp, Artificial Sequence
  Description of Artificial Sequence Synthetic oligonucleotide
cagcagctccgtctccaaaaagg

SEQ ID NO: 673 - DNA, 23 bp, Artificial Sequence
  Description of Artificial Sequence Synthetic oligonucleotide
agcagctccgtctccaaaaaggg

SEQ ID NO: 674 - DNA, 23 bp, Artificial Sequence
  Description of Artificial Sequence Synthetic oligonucleotide
tccaaaaagggagttgccgatgg

SEQ ID NO: 675 - DNA, 23 bp, Artificial Sequence
  Description of Artificial Sequence Synthetic oligonucleotide
tgccgatggcgttgtcgagatgg

SEQ ID NO: 676 - DNA, 23 bp, Artificial Sequence
  Description of Artificial Sequence Synthetic oligonucleotide
gccgatggcgttgtcgagatggg

SEQ ID NO: 677 - DNA, 23 bp, Artificial Sequence
  Description of Artificial Sequence Synthetic oligonucleotide
ttgtcgagatgggactcatttgg

SEQ ID NO: 678 - DNA, 23 bp, Artificial Sequence
  Description of Artificial Sequence Synthetic oligonucleotide
tggtccgcgaagcccgatgatgg

SEQ ID NO: 679 - DNA, 23 bp, Artificial Sequence
  Description of Artificial Sequence Synthetic oligonucleotide
aagcccgatgatggtgagctcgg

SEQ ID NO: 680 - DNA, 23 bp, Artificial Sequence
  Description of Artificial Sequence Synthetic oligonucleotide
ccactagaacacgagcaccgcgg

SEQ ID NO: 681 - DNA, 23 bp, Artificial Sequence
  Description of Artificial Sequence Synthetic oligonucleotide
acgagcaccgcggttgttctcgg

SEQ ID NO: 682 - DNA, 23 bp, Artificial Sequence
  Description of Artificial Sequence Synthetic oligonucleotide
gttgttctcggcgagatccttgg

SEQ ID NO: 683 - DNA, 23 bp, Artificial Sequence
  Description of Artificial Sequence Synthetic oligonucleotide
gacgcaatacttgttgacattgg

SEQ ID NO: 684 - DNA, 23 bp, Artificial Sequence
  Description of Artificial Sequence Synthetic oligonucleotide
aatacttgttgacattggcgcgg

SEQ ID NO: 685 - DNA, 23 bp, Artificial Sequence
  Description of Artificial Sequence Synthetic oligonucleotide
acttgttgacattggcgcggagg

SEQ ID NO: 686 - DNA, 23 bp, Artificial Sequence
  Description of Artificial Sequence Synthetic oligonucleotide
tgacattggcgcggaggccgagg

SEQ ID NO: 687 - DNA, 23 bp, Artificial Sequence
  Description of Artificial Sequence Synthetic oligonucleotide
ggcgcggaggccgaggagcttgg

SEQ ID NO: 688 - DNA, 23 bp, Artificial Sequence
  Description of Artificial Sequence Synthetic oligonucleotide
ggccgaggagcttggcgcactgg

SEQ ID NO: 689 - DNA, 23 bp, Artificial Sequence
  Description of Artificial Sequence Synthetic oligonucleotide
gcgcactggaagtcagcgctagg

SEQ ID NO: 690 - DNA, 23 bp, Artificial Sequence
  Description of Artificial Sequence Synthetic oligonucleotide
gctaggcatgtcgacaccgcagg

SEQ ID NO: 691 - DNA, 23 bp, Artificial Sequence
  Description of Artificial Sequence Synthetic oligonucleotide
aggcatgtcgacaccgcaggagg

SEQ ID NO: 692 - DNA, 23 bp, Artificial Sequence
  Description of Artificial Sequence Synthetic oligonucleotide
agatgagtgatctcagactttgg

SEQ ID NO: 693 - DNA, 23 bp, Artificial Sequence
  Description of Artificial Sequence Synthetic oligonucleotide
tggacgtccccattcagcgatgg

SEQ ID NO: 694 - DNA, 23 bp, Artificial Sequence
  Description of Artificial Sequence Synthetic oligonucleotide
gatggctttgacagctgcttcgg

SEQ ID NO: 695 - DNA, 23 bp, Artificial Sequence
  Description of Artificial Sequence Synthetic oligonucleotide
tgacagctgcttcggctcccagg

SEQ ID NO: 696 - DNA, 23 bp, Artificial Sequence
  Description of Artificial Sequence Synthetic oligonucleotide
gacagctgcttcggctcccaggg

SEQ ID NO: 697 - DNA, 23 bp, Artificial Sequence
  Description of Artificial Sequence Synthetic oligonucleotide
gctgcttcggctcccagggccgg

SEQ ID NO: 698 - DNA, 23 bp, Artificial Sequence
  Description of Artificial Sequence Synthetic oligonucleotide
gctcccagggccggcaccccagg

SEQ ID NO: 699 - DNA, 23 bp, Artificial Sequence
  Description of Artificial Sequence Synthetic oligonucleotide
ctcccagggccggcaccccaggg

SEQ ID NO: 700 - DNA, 23 bp, Artificial Sequence
  Description of Artificial Sequence Synthetic oligonucleotide
ccccagggacgcaaatatcctgg

SEQ ID NO: 701 - DNA, 23 bp, Artificial Sequence
  Description of Artificial Sequence Synthetic oligonucleotide
cagggacgcaaatatcctggcgg

SEQ ID NO: 702 - DNA, 23 bp, Artificial Sequence
  Description of Artificial Sequence Synthetic oligonucleotide
aaatatcctggcggtcgttgagg

SEQ ID NO: 703 - DNA, 23 bp, Artificial Sequence
  Description of Artificial Sequence Synthetic oligonucleotide
tcctggcggtcgttgaggctcgg

SEQ ID NO: 704 - DNA, 23 bp, Artificial Sequence
  Description of Artificial Sequence Synthetic oligonucleotide
ctcgtcgaatgaagtaatgttgg

SEQ ID NO: 705 - DNA, 23 bp, Artificial Sequence
  Description of Artificial Sequence Synthetic oligonucleotide
tcgtcgaatgaagtaatgttggg

SEQ ID NO: 706 - DNA, 23 bp, Artificial Sequence
  Description of Artificial Sequence Synthetic oligonucleotide
taatgttgggaaacttcttgagg

SEQ ID NO: 707 - DNA, 23 bp, Artificial Sequence
  Description of Artificial Sequence Synthetic oligonucleotide
ctcgactttgtgctcgctgttgg

SEQ ID NO: 708 - DNA, 23 bp, Artificial Sequence
  Description of Artificial Sequence Synthetic oligonucleotide
gactttgtgctcgctgttggtgg

SEQ ID NO: 709 - DNA, 23 bp, Artificial Sequence
  Description of Artificial Sequence Synthetic oligonucleotide
tgtgctcgctgttggtggcgcgg

SEQ ID NO: 710 - DNA, 23 bp, Artificial Sequence
  Description of Artificial Sequence Synthetic oligonucleotide
gcggaagtagaagtccgcatagg

SEQ ID NO: 711 - DNA, 23 bp, Artificial Sequence
  Description of Artificial Sequence Synthetic oligonucleotide
aagtccgcataggtgtcctgagg

SEQ ID NO: 712 - DNA, 23 bp, Artificial Sequence
  Description of Artificial Sequence Synthetic oligonucleotide
gtgtcctgaggaaagatatgagg

SEQ ID NO: 713 - DNA, 23 bp, Artificial Sequence
  Description of Artificial Sequence Synthetic oligonucleotide
tcctgaggaaagatatgaggagg

SEQ ID NO: 714 - DNA, 23 bp, Artificial Sequence
  Description of Artificial Sequence Synthetic oligonucleotide
cctgaggaaagatatgaggaggg

SEQ ID NO: 715 - DNA, 23 bp, Artificial Sequence
  Description of Artificial Sequence Synthetic oligonucleotide
aggagggtgagctgttccgatgg

SEQ ID NO: 716 - DNA, 23 bp, Artificial Sequence
  Description of Artificial Sequence Synthetic oligonucleotide
tccgatggccatcactgttgcgg

SEQ ID NO: 717 - DNA, 23 bp, Artificial Sequence
  Description of Artificial Sequence Synthetic oligonucleotide
ttgcggttccatctgctttttgg

SEQ ID NO: 718 - DNA, 23 bp, Artificial Sequence
  Description of Artificial Sequence Synthetic oligonucleotide
tgcggttccatctgctttttggg

SEQ ID NO: 719 - DNA, 23 bp, Artificial Sequence
  Description of Artificial Sequence Synthetic oligonucleotide
catctgctttttgggcctttcgg

SEQ ID NO: 720 - DNA, 23 bp, Artificial Sequence
  Description of Artificial Sequence Synthetic oligonucleotide
tgctttttgggcctttcggatgg

SEQ ID NO: 721 - DNA, 23 bp, Artificial Sequence
  Description of Artificial Sequence Synthetic oligonucleotide
gcctgcacatcctccatcactgg

SEQ ID NO: 722 - DNA, 23 bp, Artificial Sequence
  Description of Artificial Sequence Synthetic oligonucleotide
ctccatcactggcgagtagttgg

SEQ ID NO: 723 - DNA, 24 bp, Artificial Sequence
  Description of Artificial Sequence Synthetic oligonucleotide
tttcctcaggacacctatgcggac

SEQ ID NO: 724 - DNA, 24 bp, Artificial Sequence
  Description of Artificial Sequence Synthetic oligonucleotide
tttcccaacattacttcattcgac

SEQ ID NO: 725 - DNA, 24 bp, Artificial Sequence
  Description of Artificial Sequence Synthetic oligonucleotide
tttgcgtccctggggtgccggccc

SEQ ID NO: 726 - DNA, 24 bp, Artificial Sequence
  Description of Artificial Sequence Synthetic oligonucleotide
tttttggagacggagctgctgcgc

SEQ ID NO: 727 - DNA, 24 bp, Artificial Sequence
  Description of Artificial Sequence Synthetic oligonucleotide
ttttggagacggagctgctgcgct

SEQ ID NO: 728 - DNA, 24 bp, Artificial Sequence
  Description of Artificial Sequence Synthetic oligonucleotide
tttggagacggagctgctgcgctg

SEQ ID NO: 729 - DNA, 24 bp, Artificial Sequence
  Description of Artificial Sequence Synthetic oligonucleotide
tttcggaccagggatgaccgttga

SEQ ID NO: 730 - DNA, 24 bp, Artificial Sequence
  Description of Artificial Sequence Synthetic oligonucleotide
tttcaacggtcatccctggtccga

SEQ ID NO: 731 - DNA, 24 bp, Artificial Sequence
  Description of Artificial Sequence Synthetic oligonucleotide
ttttcttctcatctcatccaatat

SEQ ID NO: 732 - DNA, 24 bp, Artificial Sequence
  Description of Artificial Sequence Synthetic oligonucleotide
tttcttctcatctcatccaatatg

SEQ ID NO: 733 - DNA, 24 bp, Artificial Sequence
  Description of Artificial Sequence Synthetic oligonucleotide
tttccgcaatcccagagcacagtt

SEQ ID NO: 734 - DNA, 24 bp, Artificial Sequence
  Description of Artificial Sequence Synthetic oligonucleotide
tttgggatcacggtctgctttgcg

SEQ ID NO: 735 - DNA, 24 bp, Artificial Sequence
  Description of Artificial Sequence Synthetic oligonucleotide
tttgcgcacacgatctcgaacata

SEQ ID NO: 736 - DNA, 24 bp, Artificial Sequence
  Description of Artificial Sequence Synthetic oligonucleotide
tttggtccgcgaagcccgatgatg

SEQ ID NO: 737 - DNA, 24 bp, Artificial Sequence
  Description of Artificial Sequence Synthetic oligonucleotide
tttggacgtccccattcagcgatg

SEQ ID NO: 738 - DNA, 24 bp, Artificial Sequence
  Description of Artificial Sequence Synthetic oligonucleotide
tttgacagctgcttcggctcccag

SEQ ID NO: 739 - DNA, 24 bp, Artificial Sequence
  Description of Artificial Sequence Synthetic oligonucleotide
tttttgcagatacgatcgaatttc

SEQ ID NO: 740 - DNA, 24 bp, Artificial Sequence
  Description of Artificial Sequence Synthetic oligonucleotide
ttttgcagatacgatcgaatttct

SEQ ID NO: 741 - DNA, 24 bp, Artificial Sequence
  Description of Artificial Sequence Synthetic oligonucleotide
tttgcagatacgatcgaatttctt

SEQ ID NO: 742 - DNA, 24 bp, Artificial Sequence
  Description of Artificial Sequence Synthetic oligonucleotide
tttctttttgagctcgactttgtg

SEQ ID NO: 743 - DNA, 24 bp, Artificial Sequence
  Description of Artificial Sequence Synthetic oligonucleotide
tttttgagctcgactttgtgctcg

SEQ ID NO: 744 - DNA, 24 bp, Artificial Sequence
  Description of Artificial Sequence Synthetic oligonucleotide
ttttgagctcgactttgtgctcgc

SEQ ID NO: 745 - DNA, 24 bp, Artificial Sequence
  Description of Artificial Sequence Synthetic oligonucleotide
tttgagctcgactttgtgctcgct

SEQ ID NO: 746 - DNA, 24 bp, Artificial Sequence
  Description of Artificial Sequence Synthetic oligonucleotide
tttgtgctcgctgttggtggcgcg

SEQ ID NO: 747 - DNA, 24 bp, Artificial Sequence
  Description of Artificial Sequence Synthetic oligonucleotide
tttttgggcctttcggatggcctg

SEQ ID NO: 748 - DNA, 24 bp, Artificial Sequence
  Description of Artificial Sequence Synthetic oligonucleotide
ttttgggcctttcggatggcctgc

SEQ ID NO: 749 - DNA, 24 bp, Artificial Sequence
  Description of Artificial Sequence Synthetic oligonucleotide
tttgggcctttcggatggcctgca

SEQ ID NO: 750 - DNA, 24 bp, Artificial Sequence
  Description of Artificial Sequence Synthetic oligonucleotide
tttcggatggcctgcacatcctcc

SEQ ID NO: 751 - DNA, 23 bp, Artificial Sequence
  Description of Artificial Sequence Synthetic oligonucleotide
ttctctctccaactactcgccag

SEQ ID NO: 752 - DNA, 23 bp, Artificial Sequence
  Description of Artificial Sequence Synthetic oligonucleotide
ctctctccaactactcgccagtg

SEQ ID NO: 753 - DNA, 23 bp, Artificial Sequence
  Description of Artificial Sequence Synthetic oligonucleotide
ctctccaactactcgccagtgat

SEQ ID NO: 754 - DNA, 23 bp, Artificial Sequence
  Description of Artificial Sequence Synthetic oligonucleotide
ctactcgccagtgatggaggatg

SEQ ID NO: 755 - DNA, 23 bp, Artificial Sequence
  Description of Artificial Sequence Synthetic oligonucleotide
ctcgccagtgatggaggatgtgc

SEQ ID NO: 756 - DNA, 23 bp, Artificial Sequence
  Description of Artificial Sequence Synthetic oligonucleotide
ctcaccctcctcatatctttcct

SEQ ID NO: 757 - DNA, 23 bp, Artificial Sequence
  Description of Artificial Sequence Synthetic oligonucleotide
ctcctcatatctttcctcaggac

SEQ ID NO: 758 - DNA, 23 bp, Artificial Sequence
  Description of Artificial Sequence Synthetic oligonucleotide
ctcatatctttcctcaggacacc

SEQ ID NO: 759 - DNA, 23 bp, Artificial Sequence
  Description of Artificial Sequence Synthetic oligonucleotide
tttcctcaggacacctatgcgga

SEQ ID NO: 760 - DNA, 23 bp, Artificial Sequence
  Description of Artificial Sequence Synthetic oligonucleotide
ttcctcaggacacctatgcggac

SEQ ID NO: 761 - DNA, 23 bp, Artificial Sequence
  Description of Artificial Sequence Synthetic oligonucleotide
ctcaggacacctatgcggacttc

SEQ ID NO: 762 - DNA, 23 bp, Artificial Sequence
  Description of Artificial Sequence Synthetic oligonucleotide
ctatgcggacttctacttccgcg

SEQ ID NO: 763 - DNA, 23 bp, Artificial Sequence
  Description of Artificial Sequence Synthetic oligonucleotide
cttctacttccgcgccaccaaca

SEQ ID NO: 764 - DNA, 23 bp, Artificial Sequence
  Description of Artificial Sequence Synthetic oligonucleotide
ttctacttccgcgccaccaacag

SEQ ID NO: 765 - DNA, 23 bp, Artificial Sequence
  Description of Artificial Sequence Synthetic oligonucleotide
ctacttccgcgccaccaacagcg

SEQ ID NO: 766 - DNA, 23 bp, Artificial Sequence
  Description of Artificial Sequence Synthetic oligonucleotide
cttccgcgccaccaacagcgagc

SEQ ID NO: 767 - DNA, 23 bp, Artificial Sequence
  Description of Artificial Sequence Synthetic oligonucleotide
ttccgcgccaccaacagcgagca

SEQ ID NO: 768 - DNA, 23 bp, Artificial Sequence
  Description of Artificial Sequence Synthetic oligonucleotide
ctcaaaaagaaattcgatcgtat

SEQ ID NO: 769 - DNA, 23 bp, Artificial Sequence
  Description of Artificial Sequence Synthetic oligonucleotide
ttcgatcgtatctgcaaaaagac

SEQ ID NO: 770 - DNA, 23 bp, Artificial Sequence
  Description of Artificial Sequence Synthetic oligonucleotide
ctgcaaaaagacaatgataggca

SEQ ID NO: 771 - DNA, 23 bp, Artificial Sequence
  Description of Artificial Sequence Synthetic oligonucleotide
ctacttcaactacgatgaggaat

SEQ ID NO: 772 - DNA, 23 bp, Artificial Sequence
  Description of Artificial Sequence Synthetic oligonucleotide
cttcaactacgatgaggaattcc

SEQ ID NO: 773 - DNA, 23 bp, Artificial Sequence
  Description of Artificial Sequence Synthetic oligonucleotide
ttcaactacgatgaggaattcct

SEQ ID NO: 774 - DNA, 23 bp, Artificial Sequence
  Description of Artificial Sequence Synthetic oligonucleotide
ctacgatgaggaattcctcaaga

SEQ ID NO: 775 - DNA, 23 bp, Artificial Sequence
  Description of Artificial Sequence Synthetic oligonucleotide
ttcctcaagaagtttcccaacat

SEQ ID NO: 776 - DNA, 23 bp, Artificial Sequence
  Description of Artificial Sequence Synthetic oligonucleotide
ctcaagaagtttcccaacattac

SEQ ID NO: 777 - DNA, 23 bp, Artificial Sequence
  Description of Artificial Sequence Synthetic oligonucleotide
tttcccaacattacttcattcga

SEQ ID NO: 778 - DNA, 23 bp, Artificial Sequence
  Description of Artificial Sequence Synthetic oligonucleotide
ttcccaacattacttcattcgac

SEQ ID NO: 779 - DNA, 23 bp, Artificial Sequence
  Description of Artificial Sequence Synthetic oligonucleotide
ttacttcattcgacgagccgagc

SEQ ID NO: 780 - DNA, 23 bp, Artificial Sequence
  Description of Artificial Sequence Synthetic oligonucleotide
cttcattcgacgagccgagcctc

SEQ ID NO: 781 - DNA, 23 bp, Artificial Sequence
  Description of Artificial Sequence Synthetic oligonucleotide
ttcattcgacgagccgagcctca

SEQ ID NO: 782 - DNA, 23 bp, Artificial Sequence
  Description of Artificial Sequence Synthetic oligonucleotide
ttcgacgagccgagcctcaacga

SEQ ID NO: 783 - DNA, 23 bp, Artificial Sequence
  Description of Artificial Sequence Synthetic oligonucleotide
ctcaacgaccgccaggatatttg

SEQ ID NO: 784 - DNA, 23 bp, Artificial Sequence
  Description of Artificial Sequence Synthetic oligonucleotide
tttgcgtccctggggtgccggcc

SEQ ID NO: 785 - DNA, 23 bp, Artificial Sequence
  Description of Artificial Sequence Synthetic oligonucleotide
ttgcgtccctggggtgccggccc

SEQ ID NO: 786 - DNA, 23 bp, Artificial Sequence
  Description of Artificial Sequence Synthetic oligonucleotide
ctggggtgccggccctgggagcc

SEQ ID NO: 787 - DNA, 23 bp, Artificial Sequence
  Description of Artificial Sequence Synthetic oligonucleotide
ctgggagccgaagcagctgtcaa

SEQ ID NO: 788 - DNA, 23 bp, Artificial Sequence
  Description of Artificial Sequence Synthetic oligonucleotide
ctgaatggggacgtccaaagtct

SEQ ID NO: 789 - DNA, 23 bp, Artificial Sequence
  Description of Artificial Sequence Synthetic oligonucleotide
ctgagatcactcatctcgtcttc

SEQ ID NO: 790 - DNA, 23 bp, Artificial Sequence
  Description of Artificial Sequence Synthetic oligonucleotide
ctcatctcgtcttctgcacctcc

SEQ ID NO: 791 - DNA, 23 bp, Artificial Sequence
  Description of Artificial Sequence Synthetic oligonucleotide
cttctgcacctcctgcggtgtcg

SEQ ID NO: 792 - DNA, 23 bp, Artificial Sequence
  Description of Artificial Sequence Synthetic oligonucleotide
ttctgcacctcctgcggtgtcga

SEQ ID NO: 793 - DNA, 23 bp, Artificial Sequence
  Description of Artificial Sequence Synthetic oligonucleotide
ctgcacctcctgcggtgtcgaca

SEQ ID NO: 794 - DNA, 23 bp, Artificial Sequence
  Description of Artificial Sequence Synthetic oligonucleotide
ctcctgcggtgtcgacatgccta

SEQ ID NO: 795 - DNA, 23 bp, Artificial Sequence
  Description of Artificial Sequence Synthetic oligonucleotide
ctgcggtgtcgacatgcctagcg

SEQ ID NO: 796 - DNA, 23 bp, Artificial Sequence
  Description of Artificial Sequence Synthetic oligonucleotide
ctagcgctgacttccagtgcgcc

SEQ ID NO: 797 - DNA, 23 bp, Artificial Sequence
  Description of Artificial Sequence Synthetic oligonucleotide
ctgacttccagtgcgccaagctc

SEQ ID NO: 798 - DNA, 23 bp, Artificial Sequence
  Description of Artificial Sequence Synthetic oligonucleotide
cttccagtgcgccaagctcctcg

SEQ ID NO: 799 - DNA, 23 bp, Artificial Sequence
  Description of Artificial Sequence Synthetic oligonucleotide
ctcctcggcctccgcgccaatgt

SEQ ID NO: 800 - DNA, 23 bp, Artificial Sequence
  Description of Artificial Sequence Synthetic oligonucleotide
ctcggcctccgcgccaatgtcaa

SEQ ID NO: 801 - DNA, 23 bp, Artificial Sequence
  Description of Artificial Sequence Synthetic oligonucleotide
ctccgcgccaatgtcaacaagta

SEQ ID NO: 802 - DNA, 23 bp, Artificial Sequence
  Description of Artificial Sequence Synthetic oligonucleotide
ttgcgtctacatgcagggatgct

SEQ ID NO: 803 - DNA, 23 bp, Artificial Sequence
  Description of Artificial Sequence Synthetic oligonucleotide
ctacatgcagggatgctacgctg

SEQ ID NO: 804 - DNA, 23 bp, Artificial Sequence
  Description of Artificial Sequence Synthetic oligonucleotide
ctacgctggcggcacagtcatgc

SEQ ID NO: 805 - DNA, 23 bp, Artificial Sequence
  Description of Artificial Sequence Synthetic oligonucleotide
ctggcggcacagtcatgcggtac

SEQ ID NO: 806 - DNA, 23 bp, Artificial Sequence
  Description of Artificial Sequence Synthetic oligonucleotide
ctcgccgagaacaaccgcggtgc

SEQ ID NO: 807 - DNA, 23 bp, Artificial Sequence
  Description of Artificial Sequence Synthetic oligonucleotide
ctcgtgttctagtggtgtgcgcc

SEQ ID NO: 808 - DNA, 23 bp, Artificial Sequence
  Description of Artificial Sequence Synthetic oligonucleotide
ttctagtggtgtgcgccgagctc

SEQ ID NO: 809 - DNA, 23 bp, Artificial Sequence
  Description of Artificial Sequence Synthetic oligonucleotide
ctagtggtgtgcgccgagctcac

SEQ ID NO: 810 - DNA, 23 bp, Artificial Sequence
  Description of Artificial Sequence Synthetic oligonucleotide
cttcgcggaccaaatgagtccca

SEQ ID NO: 811 - DNA, 23 bp, Artificial Sequence
  Description of Artificial Sequence Synthetic oligonucleotide
ttcgcggaccaaatgagtcccat

SEQ ID NO: 812 - DNA, 23 bp, Artificial Sequence
  Description of Artificial Sequence Synthetic oligonucleotide
ctcgacaacgccatcggcaactc

SEQ ID NO: 813 - DNA, 23 bp, Artificial Sequence
  Description of Artificial Sequence Synthetic oligonucleotide
ctccctttttggagacggagctg

SEQ ID NO: 814 - DNA, 23 bp, Artificial Sequence
  Description of Artificial Sequence Synthetic oligonucleotide
ctttttggagacggagctgctgc

SEQ ID NO: 815 - DNA, 23 bp, Artificial Sequence
  Description of Artificial Sequence Synthetic oligonucleotide
tttttggagacggagctgctgcg

SEQ ID NO: 816 - DNA, 23 bp, Artificial Sequence
  Description of Artificial Sequence Synthetic oligonucleotide
ttttggagacggagctgctgcgc

SEQ ID NO: 817 - DNA, 23 bp, Artificial Sequence
  Description of Artificial Sequence Synthetic oligonucleotide
tttggagacggagctgctgcgct

SEQ ID NO: 818 - DNA, 23 bp, Artificial Sequence
  Description of Artificial Sequence Synthetic oligonucleotide
ttggagacggagctgctgcgctg

SEQ ID NO: 819 - DNA, 23 bp, Artificial Sequence
  Description of Artificial Sequence Synthetic oligonucleotide
ctgctgcgctgattgtcgggtca

SEQ ID NO: 820 - DNA, 23 bp, Artificial Sequence
  Description of Artificial Sequence Synthetic oligonucleotide
ctgcgctgattgtcgggtcagac

SEQ ID NO: 821 - DNA, 23 bp, Artificial Sequence
  Description of Artificial Sequence Synthetic oligonucleotide
ctgattgtcgggtcagaccccat

SEQ ID NO: 822 - DNA, 23 bp, Artificial Sequence
  Description of Artificial Sequence Synthetic oligonucleotide
ttgtcgggtcagaccccatcatt

SEQ ID NO: 823 - DNA, 23 bp, Artificial Sequence
  Description of Artificial Sequence Synthetic oligonucleotide
ttggcgtcgagaagcctatgttc

SEQ ID NO: 824 - DNA, 23 bp, Artificial Sequence
  Description of Artificial Sequence Synthetic oligonucleotide
ctatgttcgagatcgtgtgcgca

SEQ ID NO: 825 - DNA, 23 bp, Artificial Sequence
  Description of Artificial Sequence Synthetic oligonucleotide
ttcgagatcgtgtgcgcaaagca

SEQ ID NO: 826 - DNA, 23 bp, Artificial Sequence
  Description of Artificial Sequence Synthetic oligonucleotide
ttatccatctccacatgagagag

SEQ ID NO: 827 - DNA, 23 bp, Artificial Sequence
  Description of Artificial Sequence Synthetic oligonucleotide
ctccacatgagagaggcaggtct

SEQ ID NO: 828 - DNA, 23 bp, Artificial Sequence
  Description of Artificial Sequence Synthetic oligonucleotide
ctaatgttctacatgagcaagga

SEQ ID NO: 829 - DNA, 23 bp, Artificial Sequence
  Description of Artificial Sequence Synthetic oligonucleotide
ttctacatgagcaaggacagccc

SEQ ID NO: 830 - DNA, 23 bp, Artificial Sequence
  Description of Artificial Sequence Synthetic oligonucleotide
ctacatgagcaaggacagccctg

SEQ ID NO: 831 - DNA, 23 bp, Artificial Sequence
  Description of Artificial Sequence Synthetic oligonucleotide
ctgagaccatctccaacaatgta

SEQ ID NO: 832 - DNA, 23 bp, Artificial Sequence
  Description of Artificial Sequence Synthetic oligonucleotide
ctccaacaatgtagaggcatgcc

SEQ ID NO: 833 - DNA, 23 bp, Artificial Sequence
  Description of Artificial Sequence Synthetic oligonucleotide
cttgtcgatgtgttcaagtcggt

SEQ ID NO: 834 - DNA, 23 bp, Artificial Sequence
  Description of Artificial Sequence Synthetic oligonucleotide
ttgtcgatgtgttcaagtcggtg

SEQ ID NO: 835 - DNA, 23 bp, Artificial Sequence
  Description of Artificial Sequence Synthetic oligonucleotide
ttcaagtcggtggggatgactcc

SEQ ID NO: 836 - DNA, 23 bp, Artificial Sequence
  Description of Artificial Sequence Synthetic oligonucleotide
ctcctcccgaggactggaactct

SEQ ID NO: 837 - DNA, 23 bp, Artificial Sequence
  Description of Artificial Sequence Synthetic oligonucleotide
ctcccgaggactggaactctctc

SEQ ID NO: 838 - DNA, 23 bp, Artificial Sequence
  Description of Artificial Sequence Synthetic oligonucleotide
ctggaactctctcttctggatcc

SEQ ID NO: 839 - DNA, 23 bp, Artificial Sequence
  Description of Artificial Sequence Synthetic oligonucleotide
ctctctcttctggatccctcacc

SEQ ID NO: 840 - DNA, 23 bp, Artificial Sequence
  Description of Artificial Sequence Synthetic oligonucleotide
ctctcttctggatccctcacccc

SEQ ID NO: 841 - DNA, 23 bp, Artificial Sequence
  Description of Artificial Sequence Synthetic oligonucleotide
cttctggatccctcaccccggcg

SEQ ID NO: 842 - DNA, 23 bp, Artificial Sequence
  Description of Artificial Sequence Synthetic oligonucleotide
ctggatccctcaccccggcggcc

SEQ ID NO: 843 - DNA, 23 bp, Artificial Sequence
  Description of Artificial Sequence Synthetic oligonucleotide
ctcaccccggcggccgagctatc

SEQ ID NO: 844 - DNA, 23 bp, Artificial Sequence
  Description of Artificial Sequence Synthetic oligonucleotide
ctatcctcgaccaggttgagtcc

SEQ ID NO: 845 - DNA, 23 bp, Artificial Sequence
  Description of Artificial Sequence Synthetic oligonucleotide
ctcgaccaggttgagtccaggct

SEQ ID NO: 846 - DNA, 23 bp, Artificial Sequence
  Description of Artificial Sequence Synthetic oligonucleotide
ttgagtccaggcttaagcttcgt

SEQ ID NO: 847 - DNA, 23 bp, Artificial Sequence
  Description of Artificial Sequence Synthetic oligonucleotide
cttaagcttcgtcccgagaagtt

SEQ ID NO: 848 - DNA, 23 bp, Artificial Sequence
  Description of Artificial Sequence Synthetic oligonucleotide
ttaagcttcgtcccgagaagttc

SEQ ID NO: 849 - DNA, 23 bp, Artificial Sequence
  Description of Artificial Sequence Synthetic oligonucleotide
cttcgtcccgagaagttcggcgc

SEQ ID NO: 850 - DNA, 23 bp, Artificial Sequence
  Description of Artificial Sequence Synthetic oligonucleotide
ttcgtcccgagaagttcggcgcg

SEQ ID NO: 851 - DNA, 23 bp, Artificial Sequence
  Description of Artificial Sequence Synthetic oligonucleotide
ttcggcgcgactcgaactgtgct

SEQ ID NO: 852 - DNA, 23 bp, Artificial Sequence
  Description of Artificial Sequence Synthetic oligonucleotide
ctcgaactgtgctctgggattgc

SEQ ID NO: 853 - DNA, 23 bp, Artificial Sequence
  Description of Artificial Sequence Synthetic oligonucleotide
ctgtgctctgggattgcggaaac

SEQ ID NO: 854 - DNA, 23 bp, Artificial Sequence
  Description of Artificial Sequence Synthetic oligonucleotide
ctctgggattgcggaaacatggt

SEQ ID NO: 855 - DNA, 23 bp, Artificial Sequence
  Description of Artificial Sequence Synthetic oligonucleotide
ctgggattgcggaaacatggtga

SEQ ID NO: 856 - DNA, 23 bp, Artificial Sequence
  Description of Artificial Sequence Synthetic oligonucleotide
ttgcggaaacatggtgagcgcgt

SEQ ID NO: 857 - DNA, 23 bp, Artificial Sequence
  Description of Artificial Sequence Synthetic oligonucleotide
ttctctacatattggatgagatg

SEQ ID NO: 858 - DNA, 23 bp, Artificial Sequence
  Description of Artificial Sequence Synthetic oligonucleotide
ctctacatattggatgagatgag

SEQ ID NO: 859 - DNA, 23 bp, Artificial Sequence
  Description of Artificial Sequence Synthetic oligonucleotide
ctacatattggatgagatgagaa

SEQ ID NO: 860 - DNA, 23 bp, Artificial Sequence
  Description of Artificial Sequence Synthetic oligonucleotide
ttggatgagatgagaagaaaatc

SEQ ID NO: 861 - DNA, 23 bp, Artificial Sequence
  Description of Artificial Sequence Synthetic oligonucleotide
ctgctgctgacggactagcaacc

SEQ ID NO: 862 - DNA, 23 bp, Artificial Sequence
  Description of Artificial Sequence Synthetic oligonucleotide
ctgctgacggactagcaacctac

SEQ ID NO: 863 - DNA, 23 bp, Artificial Sequence
  Description of Artificial Sequence Synthetic oligonucleotide
ctgacggactagcaacctacgga

SEQ ID NO: 864 - DNA, 23 bp, Artificial Sequence
  Description of Artificial Sequence Synthetic oligonucleotide
ctagcaacctacggagaggggct

SEQ ID NO: 865 - DNA, 23 bp, Artificial Sequence
  Description of Artificial Sequence Synthetic oligonucleotide
cttgcttggtttcggaccaggga

SEQ ID NO: 866 - DNA, 23 bp, Artificial Sequence
  Description of Artificial Sequence Synthetic oligonucleotide
ttgcttggtttcggaccagggat

SEQ ID NO: 867 - DNA, 23 bp, Artificial Sequence
  Description of Artificial Sequence Synthetic oligonucleotide
cttggtttcggaccagggatgac

SEQ ID NO: 868 - DNA, 23 bp, Artificial Sequence
  Description of Artificial Sequence Synthetic oligonucleotide
ttggtttcggaccagggatgacc

SEQ ID NO: 869 - DNA, 23 bp, Artificial Sequence
  Description of Artificial Sequence Synthetic oligonucleotide
tttcggaccagggatgaccgttg

SEQ ID NO: 870 - DNA, 23 bp, Artificial Sequence
  Description of Artificial Sequence Synthetic oligonucleotide
ttcggaccagggatgaccgttga

SEQ ID NO: 871 - DNA, 23 bp, Artificial Sequence
  Description of Artificial Sequence Synthetic oligonucleotide
ttgaaactatccttctccacagc

SEQ ID NO: 872 - DNA, 23 bp, Artificial Sequence
  Description of Artificial Sequence Synthetic oligonucleotide
ctatccttctccacagcctgccc

SEQ ID NO: 873 - DNA, 23 bp, Artificial Sequence
  Description of Artificial Sequence Synthetic oligonucleotide
cttctccacagcctgcccactgt

SEQ ID NO: 874 - DNA, 23 bp, Artificial Sequence
  Description of Artificial Sequence Synthetic oligonucleotide
ttctccacagcctgcccactgtg

SEQ ID NO: 875 - DNA, 23 bp, Artificial Sequence
  Description of Artificial Sequence Synthetic oligonucleotide
ctccacagcctgcccactgtgta

SEQ ID NO: 876 - DNA, 23 bp, Artificial Sequence
  Description of Artificial Sequence Synthetic oligonucleotide
ttacacagtgggcaggctgtgga

SEQ ID NO: 877 - DNA, 23 bp, Artificial Sequence
  Description of Artificial Sequence Synthetic oligonucleotide
ctgtggagaaggatagtttcaac

SEQ ID NO: 878 - DNA, 23 bp, Artificial Sequence
  Description of Artificial Sequence Synthetic oligonucleotide
tttcaacggtcatccctggtccg

SEQ ID NO: 879 - DNA, 23 bp, Artificial Sequence
  Description of Artificial Sequence Synthetic oligonucleotide
ttcaacggtcatccctggtccga

SEQ ID NO: 880 - DNA, 23 bp, Artificial Sequence
  Description of Artificial Sequence Synthetic oligonucleotide
ctggtccgaaaccaagcaagaca

SEQ ID NO: 881 - DNA, 23 bp, Artificial Sequence
  Description of Artificial Sequence Synthetic oligonucleotide
ctccagcccctctccgtaggttg

SEQ ID NO: 882 - DNA, 23 bp, Artificial Sequence
  Description of Artificial Sequence Synthetic oligonucleotide
ctctccgtaggttgctagtccgt

SEQ ID NO: 883 - DNA, 23 bp, Artificial Sequence
  Description of Artificial Sequence Synthetic oligonucleotide
ctccgtaggttgctagtccgtca

SEQ ID NO: 884 - DNA, 23 bp, Artificial Sequence
  Description of Artificial Sequence Synthetic oligonucleotide
ttgctagtccgtcagcagcagat

SEQ ID NO: 885 - DNA, 23 bp, Artificial Sequence
  Description of Artificial Sequence Synthetic oligonucleotide
ctagtccgtcagcagcagatttt

SEQ ID NO: 886 - DNA, 23 bp, Artificial Sequence
  Description of Artificial Sequence Synthetic oligonucleotide
ttttcttctcatctcatccaata

SEQ ID NO: 887 - DNA, 23 bp, Artificial Sequence
  Description of Artificial Sequence Synthetic oligonucleotide
tttcttctcatctcatccaatat

SEQ ID NO: 888 - DNA, 23 bp, Artificial Sequence
  Description of Artificial Sequence Synthetic oligonucleotide
ttcttctcatctcatccaatatg

SEQ ID NO: 889 - DNA, 23 bp, Artificial Sequence
  Description of Artificial Sequence Synthetic oligonucleotide
cttctcatctcatccaatatgta

SEQ ID NO: 890 - DNA, 23 bp, Artificial Sequence
  Description of Artificial Sequence Synthetic oligonucleotide
ttctcatctcatccaatatgtag

SEQ ID NO: 891 - DNA, 23 bp, Artificial Sequence
  Description of Artificial Sequence Synthetic oligonucleotide
ctcatctcatccaatatgtagag

SEQ ID NO: 892 - DNA, 23 bp, Artificial Sequence
  Description of Artificial Sequence Synthetic oligonucleotide
ctcatccaatatgtagagaacac

SEQ ID NO: 893 - DNA, 23 bp, Artificial Sequence
  Description of Artificial Sequence Synthetic oligonucleotide
ctcaccatgtttccgcaatccca

SEQ ID NO: 894 - DNA, 23 bp, Artificial Sequence
  Description of Artificial Sequence Synthetic oligonucleotide
tttccgcaatcccagagcacagt

SEQ ID NO: 895 - DNA, 23 bp, Artificial Sequence
  Description of Artificial Sequence Synthetic oligonucleotide
ttccgcaatcccagagcacagtt

SEQ ID NO: 896 - DNA, 23 bp, Artificial Sequence
  Description of Artificial Sequence Synthetic oligonucleotide
ttcgagtcgcgccgaacttctcg

SEQ ID NO: 897 - DNA, 23 bp, Artificial Sequence
  Description of Artificial Sequence Synthetic oligonucleotide
cttctcgggacgaagcttaagcc

SEQ ID NO: 898 - DNA, 23 bp, Artificial Sequence
  Description of Artificial Sequence Synthetic oligonucleotide
ttctcgggacgaagcttaagcct

SEQ ID NO: 899 - DNA, 23 bp, Artificial Sequence
  Description of Artificial Sequence Synthetic oligonucleotide
cttaagcctggactcaacctggt

SEQ ID NO: 900 - DNA, 23 bp, Artificial Sequence
  Description of Artificial Sequence Synthetic oligonucleotide
ttaagcctggactcaacctggtc

SEQ ID NO: 901 - DNA, 23 bp, Artificial Sequence
  Description of Artificial Sequence Synthetic oligonucleotide
ctggactcaacctggtcgaggat

SEQ ID NO: 902 - DNA, 23 bp, Artificial Sequence
  Description of Artificial Sequence Synthetic oligonucleotide
ctcaacctggtcgaggatagctc

SEQ ID NO: 903 - DNA, 23 bp, Artificial Sequence
  Description of Artificial Sequence Synthetic oligonucleotide
ctggtcgaggatagctcggccgc

SEQ ID NO: 904 - DNA, 23 bp, Artificial Sequence
  Description of Artificial Sequence Synthetic oligonucleotide
ctcggccgccggggtgagggatc

SEQ ID NO: 905 - DNA, 23 bp, Artificial Sequence
  Description of Artificial Sequence Synthetic oligonucleotide
ttccagtcctcgggaggagtcat

SEQ ID NO: 906 - DNA, 23 bp, Artificial Sequence
  Description of Artificial Sequence Synthetic oligonucleotide
ctcgggaggagtcatccccaccg

SEQ ID NO: 907 - DNA, 23 bp, Artificial Sequence
  Description of Artificial Sequence Synthetic oligonucleotide
cttgaacacatcgacaaggcatg

SEQ ID NO: 908 - DNA, 23 bp, Artificial Sequence
  Description of Artificial Sequence Synthetic oligonucleotide
ttgaacacatcgacaaggcatgc

SEQ ID NO: 909 - DNA, 23 bp, Artificial Sequence
  Description of Artificial Sequence Synthetic oligonucleotide
ctctacattgttggagatggtct

SEQ ID NO: 910 - DNA, 23 bp, Artificial Sequence
  Description of Artificial Sequence Synthetic oligonucleotide
ctacattgttggagatggtctca

SEQ ID NO: 911 - DNA, 23 bp, Artificial Sequence
  Description of Artificial Sequence Synthetic oligonucleotide
ttgttggagatggtctcagggct

SEQ ID NO: 912 - DNA, 23 bp, Artificial Sequence
  Description of Artificial Sequence Synthetic oligonucleotide
ttggagatggtctcagggctgtc

SEQ ID NO: 913 - DNA, 23 bp, Artificial Sequence
  Description of Artificial Sequence Synthetic oligonucleotide
ctcagggctgtccttgctcatgt

SEQ ID NO: 914 - DNA, 23 bp, Artificial Sequence
  Description of Artificial Sequence Synthetic oligonucleotide
ctgtccttgctcatgtagaacat

SEQ ID NO: 915 - DNA, 23 bp, Artificial Sequence
  Description of Artificial Sequence Synthetic oligonucleotide
cttgctcatgtagaacattagac

SEQ ID NO: 916 - DNA, 23 bp, Artificial Sequence
  Description of Artificial Sequence Synthetic oligonucleotide
ttgctcatgtagaacattagacc

SEQ ID NO: 917 - DNA, 23 bp, Artificial Sequence
  Description of Artificial Sequence Synthetic oligonucleotide
ctcatgtagaacattagacctgc

SEQ ID NO: 918 - DNA, 23 bp, Artificial Sequence
  Description of Artificial Sequence Synthetic oligonucleotide
ctctctcatgtggagatggataa

SEQ ID NO: 919 - DNA, 23 bp, Artificial Sequence
  Description of Artificial Sequence Synthetic oligonucleotide
ctctcatgtggagatggataaca

SEQ ID NO: 920 - DNA, 23 bp, Artificial Sequence
  Description of Artificial Sequence Synthetic oligonucleotide
ctcatgtggagatggataacatc

SEQ ID NO: 921 - DNA, 23 bp, Artificial Sequence
  Description of Artificial Sequence Synthetic oligonucleotide
cttcactgtttgggatcacggtc

SEQ ID NO: 922 - DNA, 23 bp, Artificial Sequence
  Description of Artificial Sequence Synthetic oligonucleotide
ttcactgtttgggatcacggtct

SEQ ID NO: 923 - DNA, 23 bp, Artificial Sequence
  Description of Artificial Sequence Synthetic oligonucleotide
ctgtttgggatcacggtctgctt

SEQ ID NO: 924 - DNA, 23 bp, Artificial Sequence
  Description of Artificial Sequence Synthetic oligonucleotide
tttgggatcacggtctgctttgc

SEQ ID NO: 925 - DNA, 23 bp, Artificial Sequence
  Description of Artificial Sequence Synthetic oligonucleotide
ttgggatcacggtctgctttgcg

SEQ ID NO: 926 - DNA, 23 bp, Artificial Sequence
  Description of Artificial Sequence Synthetic oligonucleotide
ctgctttgcgcacacgatctcga

SEQ ID NO: 927 - DNA, 23 bp, Artificial Sequence
  Description of Artificial Sequence Synthetic oligonucleotide
ctttgcgcacacgatctcgaaca

SEQ ID NO: 928 - DNA, 23 bp, Artificial Sequence
  Description of Artificial Sequence Synthetic oligonucleotide
tttgcgcacacgatctcgaacat

SEQ ID NO: 929 - DNA, 23 bp, Artificial Sequence
  Description of Artificial Sequence Synthetic oligonucleotide
ttgcgcacacgatctcgaacata

SEQ ID NO: 930 - DNA, 23 bp, Artificial Sequence
  Description of Artificial Sequence Synthetic oligonucleotide
ctcgaacataggcttctcgacgc

SEQ ID NO: 931 - DNA, 23 bp, Artificial Sequence
  Description of Artificial Sequence Synthetic oligonucleotide
cttctcgacgccaatgatggggt

SEQ ID NO: 932 - DNA, 23 bp, Artificial Sequence
  Description of Artificial Sequence Synthetic oligonucleotide
ttctcgacgccaatgatggggtc

SEQ ID NO: 933 - DNA, 23 bp, Artificial Sequence
  Description of Artificial Sequence Synthetic oligonucleotide
ctcgacgccaatgatggggtctg

SEQ ID NO: 934 - DNA, 23 bp, Artificial Sequence
  Description of Artificial Sequence Synthetic oligonucleotide
ctgacccgacaatcagcgcagca

SEQ ID NO: 935 - DNA, 23 bp, Artificial Sequence
  Description of Artificial Sequence Synthetic oligonucleotide
ctccgtctccaaaaagggagttg

SEQ ID NO: 936 - DNA, 23 bp, Artificial Sequence
  Description of Artificial Sequence Synthetic oligonucleotide
ctccaaaaagggagttgccgatg

SEQ ID NO: 937 - DNA, 23 bp, Artificial Sequence
  Description of Artificial Sequence Synthetic oligonucleotide
ttgccgatggcgttgtcgagatg

SEQ ID NO: 938 - DNA, 23 bp, Artificial Sequence
  Description of Artificial Sequence Synthetic oligonucleotide
ctcatttggtccgcgaagcccga

SEQ ID NO: 939 - DNA, 23 bp, Artificial Sequence
  Description of Artificial Sequence Synthetic oligonucleotide
tttggtccgcgaagcccgatgat

SEQ ID NO: 940 - DNA, 23 bp, Artificial Sequence
  Description of Artificial Sequence Synthetic oligonucleotide
ttggtccgcgaagcccgatgatg

SEQ ID NO: 941 - DNA, 23 bp, Artificial Sequence
  Description of Artificial Sequence Synthetic oligonucleotide
ctcggcgcacaccactagaacac

SEQ ID NO: 942 - DNA, 23 bp, Artificial Sequence
  Description of Artificial Sequence Synthetic oligonucleotide
ctagaacacgagcaccgcggttg

SEQ ID NO: 943 - DNA, 23 bp, Artificial Sequence
  Description of Artificial Sequence Synthetic oligonucleotide
ttgttctcggcgagatccttggc

SEQ ID NO: 944 - DNA, 23 bp, Artificial Sequence
  Description of Artificial Sequence Synthetic oligonucleotide
ttctcggcgagatccttggcgta

SEQ ID NO: 945 - DNA, 23 bp, Artificial Sequence
  Description of Artificial Sequence Synthetic oligonucleotide
ctcggcgagatccttggcgtacc

SEQ ID NO: 946 - DNA, 23 bp, Artificial Sequence
  Description of Artificial Sequence Synthetic oligonucleotide
cttggcgtaccgcatgactgtgc

SEQ ID NO: 947 - DNA, 23 bp, Artificial Sequence
  Description of Artificial Sequence Synthetic oligonucleotide
ttggcgtaccgcatgactgtgcc

SEQ ID NO: 948 - DNA, 23 bp, Artificial Sequence
  Description of Artificial Sequence Synthetic oligonucleotide
ctgtgccgccagcgtagcatccc

SEQ ID NO: 949 - DNA, 23 bp, Artificial Sequence
  Description of Artificial Sequence Synthetic oligonucleotide
ctgcatgtagacgcaatacttgt

SEQ ID NO: 950 - DNA, 23 bp, Artificial Sequence
  Description of Artificial Sequence Synthetic oligonucleotide
cttgttgacattggcgcggaggc

SEQ ID NO: 951 - DNA, 23 bp, Artificial Sequence
  Description of Artificial Sequence Synthetic oligonucleotide
ttgttgacattggcgcggaggcc

SEQ ID NO: 952 - DNA, 23 bp, Artificial Sequence
  Description of Artificial Sequence Synthetic oligonucleotide
ttgacattggcgcggaggccgag

SEQ ID NO: 953 - DNA, 23 bp, Artificial Sequence
  Description of Artificial Sequence Synthetic oligonucleotide
ttggcgcggaggccgaggagctt

SEQ ID NO: 954 - DNA, 23 bp, Artificial Sequence
  Description of Artificial Sequence Synthetic oligonucleotide
cttggcgcactggaagtcagcgc

SEQ ID NO: 955 - DNA, 23 bp, Artificial Sequence
  Description of Artificial Sequence Synthetic oligonucleotide
ttggcgcactggaagtcagcgct

SEQ ID NO: 956 - DNA, 23 bp, Artificial Sequence
  Description of Artificial Sequence Synthetic oligonucleotide
ctggaagtcagcgctaggcatgt

SEQ ID NO: 957 - DNA, 23 bp, Artificial Sequence
  Description of Artificial Sequence Synthetic oligonucleotide
ctaggcatgtcgacaccgcagga

SEQ ID NO: 958 - DNA, 23 bp, Artificial Sequence
  Description of Artificial Sequence Synthetic oligonucleotide
ctcagactttggacgtccccatt

SEQ ID NO: 959 - DNA, 23 bp, Artificial Sequence
  Description of Artificial Sequence Synthetic oligonucleotide
ctttggacgtccccattcagcga

SEQ ID NO: 960 - DNA, 23 bp, Artificial Sequence
  Description of Artificial Sequence Synthetic oligonucleotide
tttggacgtccccattcagcgat

SEQ ID NO: 961 - DNA, 23 bp, Artificial Sequence
  Description of Artificial Sequence Synthetic oligonucleotide
ttggacgtccccattcagcgatg

SEQ ID NO: 962 - DNA, 23 bp, Artificial Sequence
  Description of Artificial Sequence Synthetic oligonucleotide
ttcagcgatggctttgacagctg

SEQ ID NO: 963 - DNA, 23 bp, Artificial Sequence
  Description of Artificial Sequence Synthetic oligonucleotide
ctttgacagctgcttcggctccc

SEQ ID NO: 964 - DNA, 23 bp, Artificial Sequence
  Description of Artificial Sequence Synthetic oligonucleotide
tttgacagctgcttcggctccca

SEQ ID NO: 965 - DNA, 23 bp, Artificial Sequence
  Description of Artificial Sequence Synthetic oligonucleotide
ttgacagctgcttcggctcccag

SEQ ID NO: 966 - DNA, 23 bp, Artificial Sequence
  Description of Artificial Sequence Synthetic oligonucleotide
ctgcttcggctcccagggccggc

SEQ ID NO: 967 - DNA, 23 bp, Artificial Sequence
  Description of Artificial Sequence Synthetic oligonucleotide
cttcggctcccagggccggcacc

SEQ ID NO: 968 - DNA, 23 bp, Artificial Sequence
  Description of Artificial Sequence Synthetic oligonucleotide
ttcggctcccagggccggcaccc

SEQ ID NO: 969 - DNA, 23 bp, Artificial Sequence
  Description of Artificial Sequence Synthetic oligonucleotide
ctggcggtcgttgaggctcggct

SEQ ID NO: 970 - DNA, 23 bp, Artificial Sequence
  Description of Artificial Sequence Synthetic oligonucleotide
ttgaggctcggctcgtcgaatga

SEQ ID NO: 971 - DNA, 23 bp, Artificial Sequence
  Description of Artificial Sequence Synthetic oligonucleotide
ctcggctcgtcgaatgaagtaat

SEQ ID NO: 972 - DNA, 23 bp, Artificial Sequence
  Description of Artificial Sequence Synthetic oligonucleotide
ttgggaaacttcttgaggaattc

SEQ ID NO: 973 - DNA, 23 bp, Artificial Sequence
  Description of Artificial Sequence Synthetic oligonucleotide
cttcttgaggaattcctcatcgt

SEQ ID NO: 974 - DNA, 23 bp, Artificial Sequence
  Description of Artificial Sequence Synthetic oligonucleotide
ttcttgaggaattcctcatcgta

SEQ ID NO: 975 - DNA, 23 bp, Artificial Sequence
  Description of Artificial Sequence Synthetic oligonucleotide
cttgaggaattcctcatcgtagt

SEQ ID NO: 976 - DNA, 23 bp, Artificial Sequence
  Description of Artificial Sequence Synthetic oligonucleotide
ttgaggaattcctcatcgtagtt

SEQ ID NO: 977 - DNA, 23 bp, Artificial Sequence
  Description of Artificial Sequence Synthetic oligonucleotide
ttcctcatcgtagttgaagtagc

SEQ ID NO: 978 - DNA, 23 bp, Artificial Sequence
  Description of Artificial Sequence Synthetic oligonucleotide
ctcatcgtagttgaagtagcgct

SEQ ID NO: 979 - DNA, 23 bp, Artificial Sequence
  Description of Artificial Sequence Synthetic oligonucleotide
ttgaagtagcgcttgcctatcat

SEQ ID NO: 980 - DNA, 23 bp, Artificial Sequence
  Description of Artificial Sequence Synthetic oligonucleotide
cttgcctatcattgtctttttgc

SEQ ID NO: 981 - DNA, 23 bp, Artificial Sequence
  Description of Artificial Sequence Synthetic oligonucleotide
ttgcctatcattgtctttttgca

SEQ ID NO: 982 - DNA, 23 bp, Artificial Sequence
  Description of Artificial Sequence Synthetic oligonucleotide
ctatcattgtctttttgcagata

SEQ ID NO: 983 - DNA, 23 bp, Artificial Sequence
  Description of Artificial Sequence Synthetic oligonucleotide
ttgtctttttgcagatacgatcg

SEQ ID NO: 984 - DNA, 23 bp, Artificial Sequence
  Description of Artificial Sequence Synthetic oligonucleotide
ctttttgcagatacgatcgaatt

SEQ ID NO: 985 - DNA, 23 bp, Artificial Sequence
  Description of Artificial Sequence Synthetic oligonucleotide
tttttgcagatacgatcgaattt

SEQ ID NO: 986 - DNA, 23 bp, Artificial Sequence
  Description of Artificial Sequence Synthetic oligonucleotide
ttttgcagatacgatcgaatttc

SEQ ID NO: 987 - DNA, 23 bp, Artificial Sequence
  Description of Artificial Sequence Synthetic oligonucleotide
tttgcagatacgatcgaatttct

SEQ ID NO: 988 - DNA, 23 bp, Artificial Sequence
  Description of Artificial Sequence Synthetic oligonucleotide
ttgcagatacgatcgaatttctt

SEQ ID NO: 989 - DNA, 23 bp, Artificial Sequence
  Description of Artificial Sequence Synthetic oligonucleotide
tttctttttgagctcgactttgt

SEQ ID NO: 990 - DNA, 23 bp, Artificial Sequence
  Description of Artificial Sequence Synthetic oligonucleotide
ttctttttgagctcgactttgtg

SEQ ID NO: 991 - DNA, 23 bp, Artificial Sequence
  Description of Artificial Sequence Synthetic oligonucleotide
ctttttgagctcgactttgtgct

SEQ ID NO: 992 - DNA, 23 bp, Artificial Sequence
  Description of Artificial Sequence Synthetic oligonucleotide
tttttgagctcgactttgtgctc

SEQ ID NO: 993 - DNA, 23 bp, Artificial Sequence
  Description of Artificial Sequence Synthetic oligonucleotide
ttttgagctcgactttgtgctcg

SEQ ID NO: 994 - DNA, 23 bp, Artificial Sequence
  Description of Artificial Sequence Synthetic oligonucleotide
tttgagctcgactttgtgctcgc

SEQ ID NO: 995 - DNA, 23 bp, Artificial Sequence
  Description of Artificial Sequence Synthetic oligonucleotide
ttgagctcgactttgtgctcgct

SEQ ID NO: 996 - DNA, 23 bp, Artificial Sequence
  Description of Artificial Sequence Synthetic oligonucleotide
ctttgtgctcgctgttggtggcg

SEQ ID NO: 997 - DNA, 23 bp, Artificial Sequence
  Description of Artificial Sequence Synthetic oligonucleotide
tttgtgctcgctgttggtggcgc

SEQ ID NO: 998 - DNA, 23 bp, Artificial Sequence
  Description of Artificial Sequence Synthetic oligonucleotide
ttgtgctcgctgttggtggcgcg

SEQ ID NO: 999 - DNA, 23 bp, Artificial Sequence
  Description of Artificial Sequence Synthetic oligonucleotide
ctcgctgttggtggcgcggaagt

SEQ ID NO: 1000 - DNA, 23 bp, Artificial Sequence
  Description of Artificial Sequence Synthetic oligonucleotide
ctgttggtggcgcggaagtagaa

SEQ ID NO: 1001 - DNA, 23 bp, Artificial Sequence
  Description of Artificial Sequence Synthetic oligonucleotide
ttggtggcgcggaagtagaagtc

SEQ ID NO: 1002 - DNA, 23 bp, Artificial Sequence
  Description of Artificial Sequence Synthetic oligonucleotide
ctgaggaaagatatgaggagggt

SEQ ID NO: 1003 - DNA, 23 bp, Artificial Sequence
  Description of Artificial Sequence Synthetic oligonucleotide
ctgttccgatggccatcactgtt

SEQ ID NO: 1004 - DNA, 23 bp, Artificial Sequence
  Description of Artificial Sequence Synthetic oligonucleotide
ttccgatggccatcactgttgcg

SEQ ID NO: 1005 - DNA, 23 bp, Artificial Sequence
  Description of Artificial Sequence Synthetic oligonucleotide
ctgttgcggttccatctgctttt

SEQ ID NO: 1006 - DNA, 23 bp, Artificial Sequence
  Description of Artificial Sequence Synthetic oligonucleotide
ttccatctgctttttgggccttt

SEQ ID NO: 1007 - DNA, 23 bp, Artificial Sequence
  Description of Artificial Sequence Synthetic oligonucleotide
ctgctttttgggcctttcggatg

SEQ ID NO: 1008 - DNA, 23 bp, Artificial Sequence
  Description of Artificial Sequence Synthetic oligonucleotide
ctttttgggcctttcggatggcc

SEQ ID NO: 1009 - DNA, 23 bp, Artificial Sequence
  Description of Artificial Sequence Synthetic oligonucleotide
tttttgggcctttcggatggcct

SEQ ID NO: 1010 - DNA, 23 bp, Artificial Sequence
  Description of Artificial Sequence Synthetic oligonucleotide
ttttgggcctttcggatggcctg

SEQ ID NO: 1011 - DNA, 23 bp, Artificial Sequence
  Description of Artificial Sequence Synthetic oligonucleotide
tttgggcctttcggatggcctgc

SEQ ID NO: 1012 - DNA, 23 bp, Artificial Sequence
  Description of Artificial Sequence Synthetic oligonucleotide
ttgggcctttcggatggcctgca

SEQ ID NO: 1013 - DNA, 23 bp, Artificial Sequence
  Description of Artificial Sequence Synthetic oligonucleotide
ctttcggatggcctgcacatcct

SEQ ID NO: 1014 - DNA, 23 bp, Artificial Sequence
  Description of Artificial Sequence Synthetic oligonucleotide
tttcggatggcctgcacatcctc

SEQ ID NO: 1015 - DNA, 23 bp, Artificial Sequence
  Description of Artificial Sequence Synthetic oligonucleotide
ttcggatggcctgcacatcctcc

SEQ ID NO: 1016 - DNA, 23 bp, Artificial Sequence
  Description of Artificial Sequence Synthetic oligonucleotide
ctgcacatcctccatcactggcg

SEQ ID NO: 1017 - DNA, 23 bp, Artificial Sequence
  Description of Artificial Sequence Synthetic oligonucleotide
ctggcgagtagttggagagagaa

SEQ ID NO: 1018 - DNA, 26 bp, Artificial Sequence
  Description of Artificial Sequence Synthetic oligonucleotide
ccaactactcgccagtgatggaggat

SEQ ID NO: 1019 - DNA, 26 bp, Artificial Sequence
  Description of Artificial Sequence Synthetic oligonucleotide
gcgctacttcaactacgatgaggaat

SEQ ID NO: 1020 - DNA, 26 bp, Artificial Sequence
  Description of Artificial Sequence Synthetic oligonucleotide
agccgagcctcaacgaccgccaggat

SEQ ID NO: 1021 - DNA, 26 bp, Artificial Sequence
  Description of Artificial Sequence Synthetic oligonucleotide
cgccaggatatttgcgtccctggggt

SEQ ID NO: 1022 - DNA, 26 bp, Artificial Sequence
  Description of Artificial Sequence Synthetic oligonucleotide
agcagctgtcaaagccatcgctgaat

SEQ ID NO: 1023 - DNA, 26 bp, Artificial Sequence
  Description of Artificial Sequence Synthetic oligonucleotide
caagtattgcgtctacatgcagggat

SEQ ID NO: 1024 - DNA, 26 bp, Artificial Sequence
  Description of Artificial Sequence Synthetic oligonucleotide
gcacagtcatgcggtacgccaaggat

SEQ ID NO: 1025 - DNA, 26 bp, Artificial Sequence
  Description of Artificial Sequence Synthetic oligonucleotide
catcgggcttcgcggaccaaatgagt

SEQ ID NO: 1026 - DNA, 26 bp, Artificial Sequence
  Description of Artificial Sequence Synthetic oligonucleotide
cggagctgctgcgctgattgtcgggt

SEQ ID NO: 1027 - DNA, 26 bp, Artificial Sequence
  Description of Artificial Sequence Synthetic oligonucleotide
gtcgatgtgttcaagtcggtggggat

SEQ ID NO: 1028 - DNA, 26 bp, Artificial Sequence
  Description of Artificial Sequence Synthetic oligonucleotide
gaggactggaactctctcttctggat

SEQ ID NO: 1029 - DNA, 26 bp, Artificial Sequence
  Description of Artificial Sequence Synthetic oligonucleotide
ccgagctatcctcgaccaggttgagt

SEQ ID NO: 1030 - DNA, 26 bp, Artificial Sequence
  Description of Artificial Sequence Synthetic oligonucleotide
gcgcgactcgaactgtgctctgggat

SEQ ID NO: 1031 - DNA, 26 bp, Artificial Sequence
  Description of Artificial Sequence Synthetic oligonucleotide
gcgcgtgtgttctctacatattggat

SEQ ID NO: 1032 - DNA, 26 bp, Artificial Sequence
  Description of Artificial Sequence Synthetic oligonucleotide
agcaacctacggagaggggctggagt

SEQ ID NO: 1033 - DNA, 26 bp, Artificial Sequence
  Description of Artificial Sequence Synthetic oligonucleotide
cctacggagaggggctggagtggggt

SEQ ID NO: 1034 - DNA, 26 bp, Artificial Sequence
  Description of Artificial Sequence Synthetic oligonucleotide
gtcttgcttggtttcggaccagggat

SEQ ID NO: 1035 - DNA, 26 bp, Artificial Sequence
  Description of Artificial Sequence Synthetic oligonucleotide
acagtgggcaggctgtggagaaggat

SEQ ID NO: 1036 - DNA, 26 bp, Artificial Sequence
  Description of Artificial Sequence Synthetic oligonucleotide
ccgcaatcccagagcacagttcgagt

SEQ ID NO: 1037 - DNA, 26 bp, Artificial Sequence
  Description of Artificial Sequence Synthetic oligonucleotide
agcctggactcaacctggtcgaggat

SEQ ID NO: 1038 - DNA, 26 bp, Artificial Sequence
  Description of Artificial Sequence Synthetic oligonucleotide
gtcgaggatagctcggccgccggggt

SEQ ID NO: 1039 - DNA, 26 bp, Artificial Sequence
  Description of Artificial Sequence Synthetic oligonucleotide
atagctcggccgccggggtgagggat

SEQ ID NO: 1040 - DNA, 26 bp, Artificial Sequence
  Description of Artificial Sequence Synthetic oligonucleotide
ggggtgagggatccagaagagagagt

SEQ ID NO: 1041 - DNA, 26 bp, Artificial Sequence
  Description of Artificial Sequence Synthetic oligonucleotide
agagagttccagtcctcgggaggagt

SEQ ID NO: 1042 - DNA, 26 bp, Artificial Sequence
  Description of Artificial Sequence Synthetic oligonucleotide
cctgcctctctcatgtggagatggat

SEQ ID NO: 1043 - DNA, 26 bp, Artificial Sequence
  Description of Artificial Sequence Synthetic oligonucleotide
aggcttctcgacgccaatgatggggt

SEQ ID NO: 1044 - DNA, 26 bp, Artificial Sequence
  Description of Artificial Sequence Synthetic oligonucleotide
agcagctccgtctccaaaaagggagt

SEQ ID NO: 1045 - DNA, 26 bp, Artificial Sequence
  Description of Artificial Sequence Synthetic oligonucleotide
caggaggtgcagaagacgagatgagt

SEQ ID NO: 1046 - DNA, 26 bp, Artificial Sequence
  Description of Artificial Sequence Synthetic oligonucleotide
ggtcgttgaggctcggctcgtcgaat

SEQ ID NO: 1047 - DNA, 26 bp, Artificial Sequence
  Description of Artificial Sequence Synthetic oligonucleotide
taatgttgggaaacttcttgaggaat

SEQ ID NO: 1048 - DNA, 26 bp, Artificial Sequence
  Description of Artificial Sequence Synthetic oligonucleotide
ttgtctttttgcagatacgatcgaat

SEQ ID NO: 1049 - DNA, 26 bp, Artificial Sequence
  Description of Artificial Sequence Synthetic oligonucleotide
gtcctgaggaaagatatgaggagggt

SEQ ID NO: 1050 - DNA, 26 bp, Artificial Sequence
  Description of Artificial Sequence Synthetic oligonucleotide
ccatctgctttttgggcctttcggat

SEQ ID NO: 1051 - DNA, 26 bp, Artificial Sequence
  Description of Artificial Sequence Synthetic oligonucleotide
ctgcacatcctccatcactggcgagt

SEQ ID NO: 1052 - DNA, 27 bp, Artificial Sequence
  Description of Artificial Sequence Synthetic oligonucleotide
tttggagacggagctgctgcgctgatt

SEQ ID NO: 1053 - DNA, 27 bp, Artificial Sequence
  Description of Artificial Sequence Synthetic oligonucleotide
gcgcgactcgaactgtgctctgggatt

SEQ ID NO: 1054 - DNA, 27 bp, Artificial Sequence
  Description of Artificial Sequence Synthetic oligonucleotide
aggttgctagtccgtcagcagcagatt

SEQ ID NO: 1055 - DNA, 27 bp, Artificial Sequence
  Description of Artificial Sequence Synthetic oligonucleotide
gagcacaaagtcgagctcaaaaagaaa

SEQ ID NO: 1056 - DNA, 27 bp, Artificial Sequence
  Description of Artificial Sequence Synthetic oligonucleotide
ctacatattggatgagatgagaagaaa

SEQ ID NO: 1057 - DNA, 28 bp, Artificial Sequence
  Description of Artificial Sequence Synthetic oligonucleotide
accctcctcatatctttcctcaggacac

SEQ ID NO: 1058 - DNA, 28 bp, Artificial Sequence
  Description of Artificial Sequence Synthetic oligonucleotide
ctacttccgcgccaccaacagcgagcac

SEQ ID NO: 1059 - DNA, 28 bp, Artificial Sequence
  Description of Artificial Sequence Synthetic oligonucleotide
ctgagatcactcatctcgtcttctgcac

SEQ ID NO: 1060 - DNA, 28 bp, Artificial Sequence
  Description of Artificial Sequence Synthetic oligonucleotide
acatgcagggatgctacgctggcggcac

SEQ ID NO: 1061 - DNA, 28 bp, Artificial Sequence
  Description of Artificial Sequence Synthetic oligonucleotide
ctacgctggcggcacagtcatgcggtac

SEQ ID NO: 1062 - DNA, 28 bp, Artificial Sequence
  Description of Artificial Sequence Synthetic oligonucleotide
atccctggtccgaaaccaagcaagacac

SEQ ID NO: 1063 - DNA, 28 bp, Artificial Sequence
  Description of Artificial Sequence Synthetic oligonucleotide
ctcatctcatccaatatgtagagaacac

SEQ ID NO: 1064 - DNA, 28 bp, Artificial Sequence
  Description of Artificial Sequence Synthetic oligonucleotide
catctcatccaatatgtagagaacacac

SEQ ID NO: 1065 - DNA, 28 bp, Artificial Sequence
  Description of Artificial Sequence Synthetic oligonucleotide
tcaccatgtttccgcaatcccagagcac

SEQ ID NO: 1066 - DNA, 28 bp, Artificial Sequence
  Description of Artificial Sequence Synthetic oligonucleotide
gaggagtcatccccaccgacttgaacac

SEQ ID NO: 1067 - DNA, 28 bp, Artificial Sequence
  Description of Artificial Sequence Synthetic oligonucleotide
gtttgggatcacggtctgctttgcgcac

SEQ ID NO: 1068 - DNA, 28 bp, Artificial Sequence
  Description of Artificial Sequence Synthetic oligonucleotide
ttgggatcacggtctgctttgcgcacac

SEQ ID NO: 1069 - DNA, 28 bp, Artificial Sequence
  Description of Artificial Sequence Synthetic oligonucleotide
aagcccgatgatggtgagctcggcgcac

SEQ ID NO: 1070 - DNA, 28 bp, Artificial Sequence
  Description of Artificial Sequence Synthetic oligonucleotide
gcccgatgatggtgagctcggcgcacac

SEQ ID NO: 1071 - DNA, 28 bp, Artificial Sequence
  Description of Artificial Sequence Synthetic oligonucleotide
gtgagctcggcgcacaccactagaacac

SEQ ID NO: 1072 - DNA, 28 bp, Artificial Sequence
  Description of Artificial Sequence Synthetic oligonucleotide
tcggcgcacaccactagaacacgagcac

SEQ ID NO: 1073 - DNA, 28 bp, Artificial Sequence
  Description of Artificial Sequence Synthetic oligonucleotide
gttgttctcggcgagatccttggcgtac

SEQ ID NO: 1074 - DNA, 28 bp, Artificial Sequence
  Description of Artificial Sequence Synthetic oligonucleotide
gtagcatccctgcatgtagacgcaatac

SEQ ID NO: 1075 - DNA, 28 bp, Artificial Sequence
  Description of Artificial Sequence Synthetic oligonucleotide
ggcgcggaggccgaggagcttggcgcac

SEQ ID NO: 1076 - DNA, 28 bp, Artificial Sequence
  Description of Artificial Sequence Synthetic oligonucleotide
tggaagtcagcgctaggcatgtcgacac

SEQ ID NO: 1077 - DNA, 28 bp, Artificial Sequence
  Description of Artificial Sequence Synthetic oligonucleotide
cagctgcttcggctcccagggccggcac

SEQ ID NO: 1078 - DNA, 28 bp, Artificial Sequence
  Description of Artificial Sequence Synthetic oligonucleotide
ttgcctatcattgtctttttgcagatac

SEQ ID NO: 1079 - DNA, 28 bp, Artificial Sequence
  Description of Artificial Sequence Synthetic oligonucleotide
ctttttgggcctttcggatggcctgcac